SEQUENCE LISTING (65 sequences)

SEQ ID NO: 1 - DNA, 11 bp, Mus musculus
aaagtaggtca

SEQ ID NO: 2 - DNA, 11 bp, Mus musculus
aaagtaggtta

SEQ ID NO: 3 - DNA, 11 bp, Mus musculus
aaagtacacga

SEQ ID NO: 4 - DNA, 21 bp, Mus musculus
tgttcctcttaatcctgccca

SEQ ID NO: 5 - DNA, 21 bp, Mus musculus
ccaacctgcacaagttccctt

SEQ ID NO: 6 - DNA, 20 bp, Mus musculus
ggcacgtatggcagcaagat

SEQ ID NO: 7 - DNA, 22 bp, Mus musculus
ccaaggaggaggattcaaactg

SEQ ID NO: 8 - DNA, 22 bp, Mus musculus
ccaatgtggtgtgggattactt

SEQ ID NO: 9 - DNA, 22 bp, Mus musculus
agtgacatccgtcttctgaaac

SEQ ID NO: 10 - DNA, 21 bp, Mus musculus
acctgtaccagaagacatacc

SEQ ID NO: 11 - DNA, 19 bp, Mus musculus
cctgcgtaagtgctcatgg

SEQ ID NO: 12 - DNA, 25 bp, Mus musculus
ccaagaaagtatggacacagacaaa

SEQ ID NO: 13 - DNA, 22 bp, Mus musculus
gcattcttgatccttccttggt

SEQ ID NO: 14 - DNA, 21 bp, Mus musculus
caagctccttggcatggtaga

SEQ ID NO: 15 - DNA, 23 bp, Mus musculus
tggatttgcaagcacaatatgaa

SEQ ID NO: 16 - DNA, 21 bp, Mus musculus
atggactacgccatgaagtct

SEQ ID NO: 17 - DNA, 19 bp, Mus musculus
cggtgctaacacgacaggg

SEQ ID NO: 18 - DNA, 23 bp, Mus musculus
ccttcagcagtcagtccataaac

SEQ ID NO: 19 - DNA, 21 bp, Mus musculus
agacatcgctggctgtgttaa

SEQ ID NO: 20 - DNA, 19 bp, Mus musculus
ctggcgtggaagtcatcgt

SEQ ID NO: 21 - DNA, 22 bp, Mus musculus
ctgtccgccattgagttctatg

SEQ ID NO: 22 - DNA, 21 bp, Mus musculus
tgtcccttcctgtgtggaaga

SEQ ID NO: 23 - DNA, 18 bp, Mus musculus
gctcccagcttggcttga

SEQ ID NO: 24 - DNA, 21 bp, Mus musculus
gggattgctgtggtagtgagc

SEQ ID NO: 25 - DNA, 22 bp, Mus musculus
ggtatggaatcaacccgttgtc

SEQ ID NO: 26 - DNA, 19 bp, Mus musculus
ggagccacgaccctagatg

SEQ ID NO: 27 - DNA, 21 bp, Mus musculus
tgccaagataaggaagccaac

SEQ ID NO: 28 - DNA, 23 bp, Mus musculus
cgtggaggtgcttaatgaccttt

SEQ ID NO: 29 - DNA, 19 bp, Mus musculus
catggcctggaatgcttga

SEQ ID NO: 30 - DNA, 23 bp, Mus musculus
caaggtcatccatgacaactttg

SEQ ID NO: 31 - DNA, 20 bp, Mus musculus
ggccatccacagtcttctgg

SEQ ID NO: 32 - DNA, 19 bp, Mus musculus
tgagccggatgcagaagga

SEQ ID NO: 33 - DNA, 21 bp, Mus musculus
gcaacatctttacactggcct

SEQ ID NO: 34 - DNA, 21 bp, Mus musculus
tcagaagacaagatcaccgga

SEQ ID NO: 35 - DNA, 21 bp, Mus musculus
gctggtgtgactgtaagtggg

SEQ ID NO: 36 - DNA, 20 bp, Mus musculus
cagacgctacgctgctatcc

SEQ ID NO: 37 - DNA, 21 bp, Mus musculus
ccctcagagtggtcgtcatca

SEQ ID NO: 38 - DNA, 20 bp, Mus musculus
gcacttggcaatcatccacg

SEQ ID NO: 39 - DNA, 23 bp, Mus musculus
gtatttcctcgaaagtctcggag

SEQ ID NO: 40 - DNA, 17 bp, Mus musculus
ccgctgtccatgaagca

SEQ ID NO: 41 - DNA, 20 bp, Mus musculus
gcagaaaagcaaaggacgtt

SEQ ID NO: 42 - DNA, 20 bp, Mus musculus
cccagctttacctgcagaag

SEQ ID NO: 43 - DNA, 20 bp, Mus musculus
atggtcgaaaggaagcctct

SEQ ID NO: 44 - DNA, 20 bp, Mus musculus
tgtgcgatgatgattcgtga

SEQ ID NO: 45 - DNA, 22 bp, Mus musculus
ggtgaaggtacgtttggtttgc

SEQ ID NO: 46 - DNA, 23 bp, Mus musculus
tatggagtgacatagagtgtgct

SEQ ID NO: 47 - DNA, 22 bp, Mus musculus
ccacttcaatccacccagaaag

SEQ ID NO: 48 - DNA, 20 bp, Mus musculus
tggatgttggcaaggcccga

SEQ ID NO: 49 - DNA, 23 bp, Mus musculus
agggccatcaaggtacaggcact

SEQ ID NO: 50 - DNA, 21 bp, Mus musculus
gaccttgtgtcctccgcttat

SEQ ID NO: 51 - DNA, 19 bp, Mus musculus
caaccgcaatttgtggctc

SEQ ID NO: 52 - DNA, 23 bp, Mus musculus
caagaataccaaagtgcgatcaa

SEQ ID NO: 53 - DNA, 26 bp, Mus musculus
gagctgggtcttttcagaataataag

SEQ ID NO: 54 - DNA, 25 bp, Mus musculus
catggtgctactgtgtaaggtgtgt

SEQ ID NO: 55 - DNA, 20 bp, Mus musculus
cacaggcgtgcactccatag

SEQ ID NO: 56 - DNA, 21 bp, Mus musculus
tgaacgcaggaggtgtgattg

SEQ ID NO: 57 - DNA, 23 bp, Mus musculus
gaggactggaagctattctcaga

SEQ ID NO: 58 - DNA, 20 bp, Mus musculus
gcacctgaccgaagacgaaa

SEQ ID NO: 59 - DNA, 19 bp, Mus musculus
gagcgatccgctgacatca

SEQ ID NO: 60 - DNA, 18 bp, Mus musculus
tcagcgccctgtgttttt

SEQ ID NO: 61 - DNA, 20 bp, Mus musculus
cgagaaccagggccgtgtag

SEQ ID NO: 62 - DNA, 22 bp, Mus musculus
ttcttgcgatacactctggtgc

SEQ ID NO: 63 - DNA, 22 bp, Mus musculus
cgggattgaatgttcttgtcgt

SEQ ID NO: 64 - DNA, 19 bp, Mus musculus
ggcagcaggcaatgcaatg

SEQ ID NO: 65 - DNA, 19 bp, Mus musculus
gggctcgtcactccagtct

(19) United States
(12) Patent Application Publication
(10) Pub. No.: US 2018/0296523 A1
(43) Pub. Date: Oct. 18, 2018

(54) USE OF POLYMETHOXYLATED FLAVONES TO AMELIORATE CIRCADIAN RHYTHM DISORDERS

(71) Applicant: THE BOARD OF REGENTS OF THE UNIVERSITY OF TEXAS SY, Austin, TX (US)

(72) Inventors: Zheng Chen, Houston, TX (US); Seung-Hee Yoo, Houston, TX (US); Joseph S. Takahashi, Dallas, TX (US)

(73) Assignee: THE BOARD OF REGENTS OF THE UNIVERSITY OF TEXAS SYSTEM, Austin, TX (US)

(21) Appl. No.: 15/911,781

(22) Filed: Mar. 5, 2018

Related U.S. Application Data
(60) Provisional application No. 62/468,227, filed on Mar. 7, 2017.

(51) Int. Cl. A61K 31/353; A61P 25/00

(52) U.S. Cl. CPC A61K 31/353; A61P 25/00

(57) ABSTRACT

Disclosed are methods comprising administering to a mammal suffering from, or at risk of suffering from, a clock-controlled disorder, such as metabolic syndrome or a constituent condition thereof, a composition comprising at least one polymethoxylated flavone. Disclosed are methods comprising administering a composition comprising at least one polymethoxylated flavone to a mammal suffering from or at risk of suffering from a sleep disorder; suffering from aging; and/or suffering from or at risk of suffering from a mood disorder. The polymethoxylated flavone may be nobiletin and/or tangeretin. The composition may also comprise other compounds, such as nicotinamide riboside and/or pterostilbene, and/or other compounds expected to improve one or more symptoms of metabolic syndrome, a sleep disorder, a mood disorder, aging, a cardiovascular disease, an immune disorder, a neurodegenerative disease, and/or a cancer.

### GOVERNMENT SUPPORT CLAUSE

This invention was made with government support under Grant Nos. AG045828 and GM114424 awarded by the United States Department of Health. The government has certain rights in the invention.

### BACKGROUND OF THE INVENTION

The present disclosure relates generally to the field of mammalian health. More particularly, it concerns the use of polymethoxylated flavones to ameliorate circadian rhythm disorders.

Virtually all living organisms on Earth have evolved an intrinsic timing system, the circadian clock, to anticipate and exploit daily environmental changes. In mammals, the clock system is hierarchically organized, with the central pacemaker in the hypothalamic suprachiasmatic nuclei (SCN) coordinating peripheral tissue clocks to perform physiological functions (Takahashi et al., 2008). The cell-autonomous molecular oscillator is the basic component of the clock system, composed of interlocked feedback loops (Dibner et al., 2010). The core loop, consisting of positive (transcriptional activators CLOCK/BMAL1 or NPAS2/BMAL1) and negative (PER1/2 and CRY1/2) arms, is responsible for generating molecular rhythms, whereas competing nuclear receptors REV-ERBs and RORs regulate Bmal1 expression to confer rhythm stability and robustness (Zhang and Kay, 2010). The molecular oscillators drive tissue-specific gene expression throughout the circadian cycle via both transcriptional and post-transcriptional mechanisms (Koike et al., 2012; Zhang et al., 2014).

A fundamental process tightly regulated by the clock system is metabolism (Asher and Schibler, 2011; Bass and Takahashi, 2010; Gerhart-Hines and Lazar, 2015; Green et al., 2008; Rutter et al., 2002), as both metabolites (Eckel-Mahan et al., 2012) and metabolic gene expression (Yang et al., 2006; Zhang et al., 2014) broadly exhibit circadian oscillations. In humans, circadian misalignment has been shown to cause metabolic disturbances such as glucose intolerance and hyperlipidemia (Roenneberg et al., 2012; Scheer et al., 2009). Genetic studies have also revealed overlapping metabolic deficiencies in clock-disrupted mice (Green et al., 2008). For example, the circadian mouse mutant Clock^{Δ19/Δ19} harboring a dominant-negative allele (Vitaterna et al. 1994; King et al. 1997) has been found to exhibit a spectrum of metabolic disorders, including obesity, hyperlipidemia, hepatic steatosis, hyperglycemia, hypoinsulinemia, and respiratory uncoupling (Marcheva et al., 2010; Shi et al., 2013; Turek et al., 2005).

Recent studies have explored the strategy of directly manipulating circadian rhythms to ameliorate the metabolic syndrome (Antoch and Kondratov, 2013; Chen et al., 2013; Farrow et al., 2012; Schroeder and Colwell, 2013). For example, time-restricted intake of high-fat diet (HFD) was shown to protect mice against metabolic disease (Hatori et al., 2012). Oscillatory amplitude of clock and metabolic gene expression was significantly enhanced in a nighttime-specific HFD regime, suggesting that the body can best expend the incoming nutrients by a concerted action of clock-associated pathways during the active period. To circumvent compliance issues inherent in behavioral interventions, a pharmacological approach involving clock-modulating small molecules was also examined (Chang et al., 2015; Chen et al., 2012, 2013; Hirota et al., 2012; Isojima et al., 2009; Meng et al., 2010; Solt et al., 2012; Wallach and Kramer, 2015). For example, small-molecule agonists acting on the REV-ERB nuclear receptors showed beneficial metabolic effects (Solt et al., 2012), suggesting modulatory compounds can improve metabolism via clock components or clock-associated mechanisms.

The inventors had previously identified several clock amplitude-enhancing small molecules (CEMs) in a high-throughput chemical screen using reporter cells with highly robust rhythms (Chen et al., 2012, 2013). When applied to cultured heterozygous Clock^{Δ19/+} PER2::Luc reporter cells in which reporter rhythms oscillate with a weaker amplitude (approximately one-third) relative to wild-type (WT) Clock+/+ cells, these CEMs were able to restore the reporter rhythm amplitude to near normal levels.

However, a need remains for clock amplitude-enhancing small molecules (CEMs), including CEMs which can be demonstrated to improve one or more symptoms of metabolic disorder in in vivo mammalian models.

### SUMMARY OF THE INVENTION

In one embodiment, the present disclosure relates to a method comprising administering, to a mammal suffering from or at risk of suffering from metabolic syndrome or a constituent condition thereof, a composition comprising at least one polymethoxylated flavone.

In one embodiment, the present disclosure relates to a method comprising administering, to a mammal suffering from or at risk of suffering from a sleep disorder, a composition comprising at least one polymethoxylated flavone.

In one embodiment, the present disclosure relates to a method comprising administering, to a mammal suffering from aging, a composition comprising at least one polymethoxylated flavone.

In one embodiment, the present disclosure relates to a method comprising administering, to a mammal suffering from or at risk of suffering from a mood disorder, a composition comprising at least one polymethoxylated flavone.

Polymethoxylated flavones, such as nobiletin and tangeretin, are clock amplitude-enhancing small molecules (CEMs) which, as confirmed by the present inventors, improve one or more symptoms of metabolic syndrome in in vivo mammalian models.

Polymethoxylated flavones, such as nobiletin and tangeretin, may improve one or more symptoms of chronic disorders that may arise or be exacerbated by a disruption in the mammal's circadian rhythm including but not limited to cardiovascular disease, immune disorders, neurodegenerative diseases, and cancers.

### BRIEF DESCRIPTION OF THE DRAWINGS

The following drawings form part of the present specification and are included to further demonstrate certain aspects of the present disclosure. The invention may be better understood by reference to one or more of these drawings in combination with the detailed description of specific embodiments presented herein.

FIG. 1A presents the chemical structure of nobiletin.

FIG. 1B shows enhancement of the PER2::Luc Clock^{Δ19/+} reporter rhythm by nobiletin in the NIH Clinical Collection.

FIG. 1C shows enhancement of the PER2::Luc Clock^{Δ19/+} reporter rhythm by nobiletin in the Microsource Spectrum Collection.

FIG. 1D shows dose-dependent effects of nobiletin on reporter rhythms from PER2rLucSV cells.

FIG. 1E shows quantification of amplitude response to nobiletin doses.

FIG. 1F shows nobiletin was not able to rescue reporter rhythms in PER2::Luc Clock^{Δ19/Δ19} fibroblast cells.

FIG. 1G shows Clock-enhancing effects of nobiletin in pituitary explants from PER2::Luc WT reporter mice.

FIG. 1H shows Clock-enhancing effects of nobiletin in pituitary explants from PER2::Luc Clock^{Δ19/+} reporter mice.

FIG. 1I shows actograms illustrating the effect of vehicle or nobiletin on circadian behavior in RC-fed WT mice (n=5).

FIG. 1J shows average wave plots summarizing wheel-running activity during 12-14 days of L:D (12 hr light, 12 hr dark) or D:D (constant darkness) for indicated genotypes (n=5).

FIG. 1K shows daily total wheel-running activity during L:D or D:D conditions for the indicated genotypes (n=5).

FIG. 2A shows structures of Tangeretin.

FIG. 2B shows identification of Tangeretin as a clock-enhancing molecule. Fully confluent PER2::LUC Clock^{Δ19/+} reporter cells were first stimulated with Fsk for 1 hr and compounds were added to the plate. Reporter luminescence was recorded over 4 days in an EnVision microplate reader.

FIG. 2C shows Clock-enhancing effects of NOB in peripheral tissues including lung explant from PER2::Luc WT reporter mice. The graphs are representative of at least three experiments.

FIG. 2D shows Clock-enhancing effects of NOB in peripheral tissues including liver explant from PER2::Luc WT reporter mice. The graphs are representative of at least three experiments.

FIG. 2E shows NOB did not enhance reporter rhythms in SCN explants from PER2::Luc WT reporter mice. Media were changed after 7 days of culture (day 0). DMSO or NOB was administered at the indicated times (Dashed line). No amplitude enhancement by NOB was observed comparing rhythms before and after NOB administration.

FIG. 2F shows mRNA level expression of Clock analyzed by real-time qPCR in PER2::LucSV cells pretreated with Forskolin for 1 hr.

FIG. 2G shows mRNA level expression of Npas2 analyzed by real-time qPCR in PER2::LucSV cells pretreated with Forskolin for 1 hr.

FIG. 2H shows mRNA level expression of Pert analyzed by real-time qPCR in PER2::LucSV cells pretreated with Forskolin for 1 hr.

FIG. 2I shows mRNA level expression of Rora analyzed by real-time qPCR in PER2::LucSV cells pretreated with Forskolin for 1 hr.

FIG. 2J shows mRNA level expression of Rev-erbβ analyzed by real-time qPCR in PER2::LucSV cells pretreated with Forskolin for 1 hr.

FIG. 2K shows mRNA level expression of Rev-erbα analyzed by real-time qPCR in PER2::LucSV cells pretreated with Forskolin for 1 hr.

FIG. 2L shows mRNA level expression of Cry2 analyzed by real-time qPCR in PER2::LucSV cells pretreated with Forskolin for 1 hr.

FIG. 2M shows mRNA level expression of Cry1 analyzed by real-time qPCR in PER2::LucSV cells pretreated with Forskolin for 1 hr.

FIG. 2N shows mRNA level expression of Bmal1 analyzed by real-time qPCR in PER2::LucSV cells pretreated with Forskolin for 1 hr.

FIG. 2O shows representative Western blots of circadian clock proteins in cells as treated in FIGS. 2F-2N.

FIG. 2P shows representative Western blots of circadian clock proteins in cells as treated in FIGS. 2F-2N.

FIG. 2Q shows quantification of Western blot (n=2-3) analysis for Per1. Data are represented as mean±SEM. *p<0.05, **p<0.01 and ***p<0.001. NOB vs. DMSO.

FIG. 2R shows quantification of Western blot (n=2-3) analysis for REV-erbα. Data are represented as mean±SEM. *p<0.05, **p<0.01 and ***p<0.001. NOB vs. DMSO.

FIG. 2S shows quantification of Western blot (n=2-3) analysis for Pert. Data are represented as mean±SEM. *p<0.05, **p<0.01 and ***p<0.001. NOB vs. DMSO.

FIG. 2T shows quantification of Western blot (n=2-3) analysis for Cry2. Data are represented as mean±SEM. *p<0.05, **p<0.01 and ***p<0.001. NOB vs. DMSO.

FIG. 2U shows quantification of Western blot (n=2-3) analysis for Bmal1. Data are represented as mean±SEM. *p<0.05, **p<0.01 and ***p<0.001. NOB vs. DMSO.

FIG. 2V shows quantification of Western blot (n=2-3) analysis for Cry1. Data are represented as mean±SEM. *p<0.05, **p<0.01 and ***p<0.001. NOB vs. DMSO.

FIG. 3A shows NOB levels in mouse plasma determined by LC-MS/MS by following administration by oral gavage at a dose of 200 mg/kg of body weight (n=3).

FIG. 3B shows NOB levels in mouse brain determined by LC-MS/MS by following administration by oral gavage at a dose of 200 mg/kg of body weight (n=3).

FIG. 3C shows NOB levels in mouse liver determined by LC-MS/MS by following administration by oral gavage at a dose of 200 mg/kg of body weight (n=3).

FIG. 3D shows body weight gains at the end of the 10-week treatment period are shown (n=8-15). In FIGS. 3B-3J, WT or Clock^{Δ19/Δ19} mutant mice were fed high-fat (HF) diet and treated with either vehicle or NOB every other day (n=8-15).

FIG. 3E shows food intake amount during dark (solid) and light (open) phases corresponding to subjective night and day. The Y axis indicates cumulative food intake for the dark or light phase (n=8-15).

FIG. 3F shows NOB reduced visceral WAT cell size. Image from three sections 200 μm apart were analyzed, representing >500 cells.

FIG. 3G shows VO2 (volume of oxygen consumed) for each group was quantified for dark (solid bars) and light (open bars) phases (n=8).

FIG. 3H shows energy expenditure was recorded over the circadian cycle (n=8).

FIG. 3I shows respiratory quotient was quantified for dark and light phases (n=8).

FIG. 3J shows weight of whole livers from HFD-fed WT and Clock^{Δ19/Δ19} mutant mice treated with Vehicle or NOB (n=8-15).

FIG. 3K shows representative images (I) of whole livers from HFD-fed WT and Clock^{Δ19/Δ19} mutant mice treated with Vehicle or NOB (n=8-15).

FIG. 3L shows histological analysis of liver after 10-week treatment. Liver were stained with oil red 0 to visualize lipid droplets.

FIG. 4A shows average body weight of WT or Clock^{Δ19/Δ19} mutant mice fed with HFD and treated with either vehicle (WT.HF.Veh or Clk.HF.Veh) or nobiletin (WT.HF.nobiletin or Clk.HF.nobiletin) for 10 weeks (n=8-15).

FIG. 4B shows representative WT or Clock^{Δ19/Δ19} mutant mice fed with HFD and treated with either vehicle (WT.HF.Veh (upper left) or Clk.HF.Veh (upper right)) or nobiletin (WT.HF.nobiletin (lower left) or Clk.HF.nobiletin (lower right)) for 10 weeks.

FIG. 4C shows daily food intake for the four groups of mice referred to in FIG. 4A (n=8-15).

FIG. 4D shows body mass composition for the four groups of mice referred to in FIG. 4A as analyzed by nuclear magnetic resonance (n=3).

FIG. 4E shows histological analysis of white adipose fat (WAT) after 10-week treatment for the four groups of mice referred to in FIG. 4A. WAT was subjected to H&E staining.

FIG. 4F shows he diurnal rhythms of VO₂ (volume of oxygen consumed) in the four groups of mice referred to in FIG. 4A (n=8).

FIG. 4G shows actograms illustrating the effect of vehicle or nobiletin on circadian behavior in HFD-fed WT mice (n=7).

FIG. 4H shows average wave plots summarizing wheel-running activity during 12-14 days of L:D or D:D for indicated genotypes (n=7).

FIG. 4I shows daily total wheel-running activity during L:D or D:D conditions for the indicated genotypes (n=7).

FIG. 4J shows actograms illustrating the effect of vehicle or nobiletin on circadian behavior in HFD-fed Clock^{Δ19/Δ19} mutant mice (n=3).

FIG. 4K shows average wave plots summarizing wheel-running activity during 10-12 days of L:D or D:D for indicated genotypes (n=3).

FIG. 4L shows daily total wheel-running activity during L:D or D:D conditions for the indicated genotypes (n=3).

FIG. 5A shows fasting blood glucose levels in HFD-fed WT with vehicle or nobiletin treatment at two opposite time points (n=8-15).

FIG. 5B shows fasting blood glucose levels in HFD-fed Clock^{Δ19/Δ19} mutant mice with vehicle or nobiletin treatment at two opposite time points (n=8-15).

FIG. 5C shows the effect of nobiletin on glucose tolerance in HFD-fed WT and Clock^{Δ19/Δ19} mutant mice as measured by glucose tolerance test (GTT) (n=8-15).

FIG. 5D shows area under the curve (AUC) of the effect of nobiletin on glucose tolerance in HFD-fed WT and Clock^{Δ19/Δ19} mutant mice as measured by glucose tolerance test (GTT).

FIG. 5E shows the effect of nobiletin on insulin tolerance in HFD-fed WT and Clock^{Δ19/Δ19} mutant mice as measured by insulin tolerance test (ITT) (n=8-15).

FIG. 5F shows the AUC of the effect of nobiletin on insulin tolerance in HFD-fed WT and Clock^{Δ19/Δ19} mutant mice as measured by insulin tolerance test (ITT).

FIG. 5G shows blood insulin levels in HFD-fed WT mice and Clock^{Δ19/Δ19} mutant mice with vehicle or nobiletin treatment (n=8-15).

FIG. 5H shows total triglyceride (TG) levels in blood after 10-week treatment (n=8-15).

FIG. 5I shows total cholesterol (TC) levels in blood after 10-week treatment (n=8-15).

FIG. 5J shows total TG levels in liver after 10-week treatment (n=8-15).

FIG. 5K shows total TC levels in liver after 10-week treatment (n=8-15).

FIG. 5L shows H&E staining of whole livers from HFD-fed WT and Clock^{Δ19/Δ19} mutant mice after 10-week treatment.

FIG. 6A shows body weight at the end of the 9-week treatment period (n=12). NOB did not effect metabolic homeostasis in regular chow-fed mice.

FIG. 6B shows body weight gain the end of the 9-week treatment period (n=12). NOB did not effect metabolic homeostasis in regular chow-fed mice.

FIG. 6C shows fasting blood glucose levels (n=12). NOB did not effect metabolic homeostasis in regular chow-fed mice.

FIG. 6D shows glucose tolerance test (GTT) (n=12). NOB did not effect metabolic homeostasis in regular chow-fed mice.

FIG. 6E shows AUC for the glucose tolerance test (GTT) of FIG. 6D. NOB did not effect metabolic homeostasis in regular chow-fed mice.

FIG. 6F shows insulin tolerance test (ITT) (n=12). NOB did not effect metabolic homeostasis in regular chow-fed mice.

FIG. 6G shows AUC for the insulin tolerance test (ITT) of FIG. 6F. NOB did not effect metabolic homeostasis in regular chow-fed mice.

FIG. 7A shows structures of Naringin (NAR) and Naringenin.

FIG. 7B shows naringin did not enhance the reporter luminescence in PER2::LucSV or PER2::LUC Clock^{Δ19/+} reporter cells.

FIG. 7C shows naringin did not enhance the reporter luminescence in PER2::LucSV or PER2::LUC Clock^{Δ19/+} reporter cells.

FIG. 7D shows naringenin did not enhance the reporter luminescence in PER2::LucSV or PER2::LUC Clock^{Δ19/+} reporter cells.

FIG. 7E shows Western blot analysis of PER2 protein in PER2::LucSV cells. Cells were treated with 5 μM of NAR or DMSO at time 0, and collected every 4 hrs up to 32 hrs followed by immunoblotting with antibody.

FIG. 7F shows real-time qPCR analysis of Per2 mRNA level in the PER2::LucSV cells treated as discussed regarding FIG. 7E.

FIG. 8A shows average body weight for HFD-fed mice treated with vehicle or NAR (WT.HF.Veh, WT.HF.NAR, Clk.HF.Veh and Clk.HF.NAR) after 10-week treatment (n=8-15).

FIG. 8B shows average body weight gain for HFD-fed mice treated with vehicle or NAR (WT.HF.Veh, WT.HF.NAR, Clk.HF.Veh and Clk.HF.NAR) after 10-week treatment (n=8-15).

FIG. 8C shows NAR reduced fasting blood glucose levels in WT mice mice at daytime and nighttime (n=8-15).

FIG. 8D shows NAR failed to reduce fasting blood glucose levels in Clock^{Δ19/Δ19} mutant mice at daytime and nighttime (n=8-15).

FIG. 8D shows NAR showed modest effects on glucose tolerance that were indistinguishable between WT and Clock^{Δ19/Δ19} mutant mice as measured by glucose tolerance test (GTT) (n=8-15).

FIG. 8F shows NAR showed modest effects on the area under curve (AUC) of glucose tolerance that were indistinguishable between WT and Clock^{Δ19/Δ19} mutant mice as measured by glucose tolerance test (GTT) (n=8-15).

FIG. 8G shows NAR showed modest effects on insulin sensitivity that were indistinguishable between WT and Clock^{Δ19/Δ19} mutant mice as measured by insulin tolerance test (ITT) (n=8-15).

FIG. 8H shows the area under curve (AUC) of ITT of FIG. 8G.

FIG. 8I shows blood insulin levels was decreased to similar extents by NAR in WT and Clock^{Δ19/Δ19} mutant mice (n=8-15).

FIG. 8J shows blood total triglyceride (TG) levels after 10-week treatment (n=8-15).

FIG. 8K shows cholesterol (TC) levels after 10-week treatment (n=8-15).

FIG. 9A shows average body weight of db/db or db/db Clock^{Δ19/Δ19} double-mutant mice fed with RC and treated with either vehicle (Db.Veh and Db.Clk.Veh) or nobiletin (Db.nobiletin and Db.Clk.nobiletin) for 10 weeks (n=6-8). The mice were 6-8 weeks old at the beginning of the treatment. Right: average body weight gain for these four groups of mice.

FIG. 9B shows average body weight gain for the four groups of mice shown in FIG. 9A.

FIG. 9C shows fasting blood glucose levels in db/db mice at two opposite time points (n=6-8).

FIG. 9D shows fasting blood glucose levels in db/db Clock^{Δ19/Δ19} double-mutant mice at two opposite time points (n=6-8).

FIG. 9E shows the effect of nobiletin on glucose tolerance in db/db and db/db Clock^{Δ19/Δ19} double-mutant mice as measured by GTT (n=6-8).

FIG. 9F shows the AUC of the effect of nobiletin on glucose tolerance in db/db and db/db Clock^{Δ19/Δ19} double-mutant mice as measured by GTT (n=6-8)

FIG. 9G shows the effect of nobiletin on insulin tolerance in db/db and db/db Clock^{Δ19/Δ19} double-mutant mice as measured by ITT (n=6-8).

FIG. 9H shows the AUC of the effect of nobiletin on insulin tolerance in db/db and db/db Clock^{Δ19/Δ19} double-mutant mice as measured by ITT (n=6-8).

FIG. 9I shows blood total TG levels in db/db and db/db Clock^{Δ19/Δ19} mice (n=6-8).

FIG. 9J shows blood total TC levels in db/db and db/db Clock^{Δ19/Δ19} mice (n=6-8).

FIG. 9K shows the effects of nobiletin on circulating insulin levels in both db/db and db/db Clock^{Δ19/Δ19} mice (n=6-8).

FIG. 10A shows a determination by Western blotting of protein and mRNA expression of clock genes in liver samples collected from HFD-fed WT mice with vehicle (WT.HF.Veh) or nobiletin (WT.HF.nobiletin) treatment. WT mice fed with RC (WT.RC.Veh) were used as controls for comparison (n=3 or 4).

FIG. 10B shows a determination by real-time qPCR of protein and mRNA expression of Cry1 in liver samples collected from HFD-fed WT mice with vehicle (WT.HF.Veh) or nobiletin (WT.HF.nobiletin) treatment. WT mice fed with RC (WT.RC.Veh) were used as controls for comparison (n=3 or 4).

FIG. 10C shows a determination by real-time qPCR of protein and mRNA expression of Bmal1 in liver samples collected from HFD-fed WT mice with vehicle (WT.HF.Veh) or nobiletin (WT.HF.nobiletin) treatment. WT mice fed with RC (WT.RC.Veh) were used as controls for comparison (n=3 or 4).

FIG. 10D shows a determination by real-time qPCR of protein and mRNA expression of Pert in liver samples collected from HFD-fed WT mice with vehicle (WT.HF.Veh) or nobiletin (WT.HF.nobiletin) treatment. WT mice fed with RC (WT.RC.Veh) were used as controls for comparison (n=3 or 4).

FIG. 10E shows a determination by real-time qPCR of protein and mRNA expression of Rev-erbα in liver samples collected from HFD-fed WT mice with vehicle (WT.HF.Veh) or nobiletin (WT.HF.nobiletin) treatment. WT mice fed with RC (WT.RC.Veh) were used as controls for comparison (n=3 or 4).

FIG. 10F shows a determination by real-time qPCR of protein and mRNA expression of Cry2 in liver samples collected from HFD-fed WT mice with vehicle (WT.HF.Veh) or nobiletin (WT.HF.nobiletin) treatment. WT mice fed with RC (WT.RC.Veh) were used as controls for comparison (n=3 or 4).

FIG. 10G shows a determination by real-time qPCR of protein and mRNA expression of RORγ in liver samples collected from HFD-fed WT mice with vehicle (WT.HF.Veh) or nobiletin (WT.HF.nobiletin) treatment. WT mice fed with RC (WT.RC.Veh) were used as controls for comparison (n=3 or 4).

FIG. 10H shows a determination by real-time qPCR of protein and mRNA expression of Rora in liver samples collected from HFD-fed WT mice with vehicle (WT.HF.Veh) or nobiletin (WT.HF.nobiletin) treatment. WT mice fed with RC (WT.RC.Veh) were used as controls for comparison (n=3 or 4).

FIG. 10I shows a determination by real-time qPCR of protein and mRNA expression of Rev-erbβ in liver samples collected from HFD-fed WT mice with vehicle (WT.HF.Veh) or nobiletin (WT.HF.nobiletin) treatment. WT mice fed with RC (WT.RC.Veh) were used as controls for comparison (n=3 or 4).

FIG. 10J shows a heatmap of microarray gene expression data indicating that the expression patterns of 56 genes were altered by HFD, and nobiletin reversed, to varying degrees, their expression to approximate RC levels in WT mouse liver at both ZT2 and ZT14 time points. Scale indicates median normalized signal intensity in relative values.

FIG. 10K shows a functional classification of 56 genes in (C) by the GO program. Percentages of genes sharing GO biological processes are shown.

FIG. 10L shows a real-time qPCR analysis of mRNA expression of clock-controlled metabolic output Cidec in the livers from treated mice.

FIG. 10M shows a real-time qPCR analysis of mRNA expression of clock-controlled metabolic output gene Pparγ in the livers from treated mice.

FIG. 10N shows a real-time qPCR analysis of mRNA expression of clock-controlled metabolic output gene CD36 in the livers from treated mice.

FIG. 10O shows a real-time qPCR analysis of mRNA expression of clock-controlled metabolic output gene Igfbp2jk in the livers from treated mice.

FIG. 10P shows a real-time qPCR analysis of mRNA expression of clock-controlled metabolic output gene Vldlr in the livers from treated mice.

FIG. 10Q shows a real-time qPCR analysis of mRNA expression of clock-controlled metabolic output gene Cyp7a1 in the livers from treated mice.

FIG. 10R shows a real-time qPCR analysis of mRNA expression of clock-controlled metabolic output gene Pdk4 in the livers from treated mice.

FIG. 10S shows a real-time qPCR analysis of mRNA expression of clock-controlled metabolic output gene Apoa4 in the livers from treated mice.

FIG. 10T shows a real-time qPCR analysis of mRNA expression of clock-controlled metabolic output gene Pkm2 in the livers from treated mice.

FIG. 10U shows a real-time qPCR analysis of mRNA expression of clock-controlled metabolic output gene Scd1 in the livers from treated mice.

FIG. 10V shows a real-time qPCR analysis of mRNA expression of clock-controlled metabolic output gene Pgc-1a in the livers from treated mice.

FIG. 10W shows a real-time qPCR analysis of mRNA expression of clock-controlled metabolic output gene Apoc2 in the livers from treated mice.

FIG. 11A shows a ratio heat map. The expression data in FIG. 10C were computed to derive fold change indicating that the expression patterns of 56 genes altered by HFD were reversed by NOB in mouse liver at both ZT2 and ZT14 time points. Scale shows fold change (ratio). NOB/HF indicates HF.NOB vs. HF.Veh expression ratio; HF/RC indicates HF.Veh vs. RC.Veh expression ratio.

FIG. 11B shows thirty-one genes show clock protein binding, whereas the remaining genes do not (blank area). Analysis was based on published ChIP-Seq results (Koike et al., 2012; Cho et al. 2012). The scale indicates magnitude of DNA occupancy as quantified by enrichment of immunoprecipitated DNA fragments bound for each transcription factor.

FIG. 11C shows RORα (Left) and RORγ (Right) mRNA expression in Hepa1-6 cells treated with control (Ctrl) or mouse RORα/γ siRNA, and vehicle (DMSO) or NOB (3 μM, 12 h) (n=4).

FIG. 11D shows highlighted within the network is RORα/γ that functions as a nodal point for genes regulated by NOB. Genes down- and up-regulated by HFD are indicated, with the intensity corresponding to fold changes.

FIG. 11E shows highlighted within the network is RORα/γ that functions as a nodal point for genes regulated by NOB. Genes down- and up-regulated by NOB are indicated, with the intensity corresponding to fold changes.

FIG. 12A shows saturation curves for 25-[3H]—OHC filter binding assays for RORα-LBD and RORγ-LBD generated with 100 ng of RORα-LBD (top) and 200 ng of RORγ-LBD (bottom) (n=3). Dissociation constant values are shown.

FIG. 12B shows Scatchard plots for the saturation curve results from FIG. 12A for 25-[3H]—OHC for RORα-LBD (top) and RORγ-LBD (bottom) corresponding to n=3. This analysis gave a dissociation constant (Kd) of 6.10 nM and a total number of binding sites (Bmax) of 100 fmol/mg of protein for RORα, and 6.67 nM and 410 fmol/mg of protein for RORγ.

FIG. 12C shows in vitro competitive radio-ligand binding assay indicating the direct binding of nobiletin (NOB) (C) but not naringin (NAR) to RORα-LBD and RORγ-LBD within the indicated dose range. Inhibitory constant values are shown.

FIG. 12D shows in vitro competitive radio-ligand binding assay indicating no direct binding of naringenin to RORα-LBD and RORγ-LBD within the indicated dose range.

FIG. 12E shows mammalian one-hybrid assays showing nobiletin interaction with ROR-LBD. Human embryonic kidney 293T cells were cotransfected with a GAL4 reporter construct with expression vectors for GAL4 DBD-RORα LBD or GAL4 DBD-RORγ LBD.

FIG. 12F shows mammalian one-hybrid assays showing a lack of naringin interaction with ROR-LBD Human embryonic kidney 293T cells were cotransfected with a GAL4 reporter construct with expression vectors for GAL4 DBD-RORα LBD or GAL4 DBD-RORγ LBD. SR1001 served as a positive control).

FIG. 12G shows nobiletin dose-dependently increased Bmal1 promoter-driven luciferase reporter expression with WT, but not mutant, RORE in the presence of RORα or RORγ in Hepa1-6 cells. Ectopic expression of REV-ERBα abolished reporter activation.

FIG. 12H shows knockdown of RORα/γ expression by siRNAs abrogated nobiletin induction of Bmal1 promoter-driven luciferase reporter expression in Hepa1-6 cells.

FIG. 12I shows knockdown of ROR□/□ expression by siRNAs abrogated nobiletin induction of Bmal1 promoter-driven luciferase reporter expression in U2OS cells.

FIG. 12J shows real-time qPCR analysis of RORα/γ target gene Apoaz1 from the same mouse liver samples as in FIG. 10A.

FIG. 12K shows real-time qPCR analysis of RORα/γ target gene Cyp7b1 from the same mouse liver samples as in FIG. 10A.

FIG. 12L shows real-time qPCR analysis of RORα/γ target gene Gck from the same mouse liver samples as in FIG. 10A.

FIG. 12M shows real-time qPCR analysis of RORα/γ target gene Glut2 from the same mouse liver samples as in FIG. 10A.

FIG. 12N shows real-time qPCR analysis of RORα/γ target gene IkBa from the same mouse liver samples as in FIG. 10A.

Throughout the figures, data are presented as mean±SEM. *p<0.05, **p<0.01, ***p<0.001, #p<0.05, and ###p<0.001.

### DESCRIPTION OF ILLUSTRATIVE EMBODIMENTS

In one embodiment, the present disclosure relates to a method, comprising administering, to a mammal suffering from or at risk of suffering from metabolic syndrome or a constituent condition thereof, a composition comprising at least one polymethoxylated flavone.

“Metabolic syndrome” is a clustering in one patient of at least three of the following medical conditions: abdominal obesity, elevated blood pressure, elevated fasting plasma glucose, high serum triglycerides, and low high-density lipoprotein (HDL). A mammal may be at risk of suffering from metabolic syndrome if the mammal has a diet high in carbohydrates, especially sugars; lives a sedentary lifestyle; and/or experiences chronic stress, among other experiences and behaviors known to the person of ordinary skill in the art. Though not to be bound by theory, one or more of the component conditions of metabolic syndrome may arise or be exacerbated by a disruption in the mammal's circadian rhythm.

Flavonoids have a general structure known in the art, comprising a fifteen-carbon skeleton comprising two phenyl rings and one heterocyclic ring. Subclasses of flavonoids include flavones, isoflavones, and neoflavones. A polymethoxylated flavone is a flavone comprising at least two methoxyl moieties.

In one embodiment, the at least one polymethoxylated flavone has formula I:

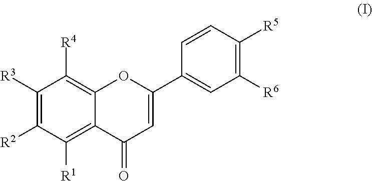

wherein R¹, R², R³, R⁴, R⁵, and R⁶ are each independently —H or —OCH₃, provided at least two of R¹, R², R³, R⁴, R⁵, and R⁶ are —OCH₃.

In a further embodiment, the at least one polymethoxylated flavone may be selected from the group consisting of nobiletin (formula II), tangeretin (formula III), and both.

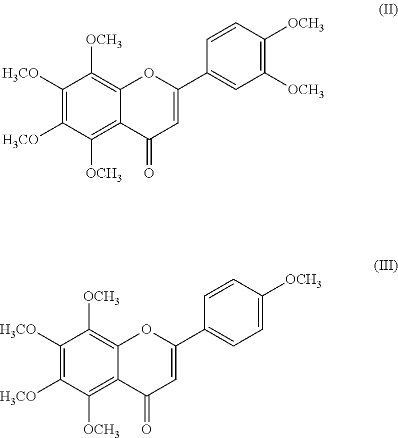 [SMILES: COc1ccc(-c2cc(=O)c3c(OC)c(OC)c(OC)c(OC)c3o2)cc1OC ; COc1ccc(-c2cc(=O)c3c(OC)c(OC)c(OC)c(OC)c3o2)cc1]

The composition may also comprise at least one carrier. The at least one carrier may be any material(s) with which the at least one polymethoxylated flavone may be mixed or combined into a desired form, and which is/are suitable for human or animal consumption. The composition may be in a form selected from the group consisting of liquids, tablets, capsules, caplets, and soluble powders, and the person of ordinary skill in the art will routinely be able to select a suitable carrier or carriers depending on the particular form desired for a given embodiment of the composition.

In one embodiment, the at least one carrier may be or include water, gelatin, or cellulose. In one embodiment, the at least one carrier may be or include microcrystalline cellulose, hypromallose, vegetable magnesium stearate, or silica.

Alternatively or in addition, the composition may also comprise a flavorant, such as a citrus flavor, a non-citrus fruit flavor, an herbal flavor, a vanilla flavor, or a chocolate flavor, among other appropriate flavorings.

Alternatively or in addition, the composition may comprise one or more ingredients other than polymethoxylated flavones expected to reduce one or more symptoms of metabolic syndrome. For example, in one embodiment, the composition may further comprise cinnamon, alone or with other ingredients expected to reduce one or more symptoms of metabolic syndrome.

Alternatively or in addition, the composition may comprise one or more ingredients expected to reduce one or more symptoms of conditions other than metabolic syndrome and/or one or more ingredients expected to improve one or more aspects of the overall health and well-being of the mammal. For example, in one embodiment, the composition may further comprise nicotinamide riboside and pterostilbene.

The composition may be administered to the mammal at any dosage and rate that provides a concentration of the at least one polymethoxylated flavone in the blood or other body tissues or fluids below a harmful level, and for any duration. Desirably, the duration may be sufficiently long for the severity of the patient's metabolic syndrome to be reduced or the patient's risk of suffering a metabolic syndrome to be reduced.

In one embodiment, administering may be at a dosage from 20 mg polymethoxylated flavone/kg body weight of the mammal to 2000 mg polymethoxylated flavone/kg body weight of the mammal and a rate from twice per day to once per week for a duration of at least one week. In a particular embodiment, administering may be at a dosage of 200 mg polymethoxylated flavone/kg body weight of the mammal, a rate of once every two days and a duration of at least ten weeks.

In a further embodiment, wherein the composition further comprises nicotinamide riboside and pterostilbene, the dosage of nicotinamide riboside may be from 1 mg nicotinamide riboside/kg body weight of the mammal to 10 mg nicotinamide riboside/kg body weight of the mammal; the dosage of pterostilbene may be from 0.2 mg pterostilbene/kg body weight of the mammal to 2.5 pterostilbene/kg body weight of the mammal; and the rate of administration may be once per day.

Any mammal for which metabolic syndrome or a risk thereof is desired to be reduced may be the subject of the method. In one embodiment, the mammal may be Homo sapiens. Other mammals for which metabolic syndrome or a risk thereof may be desired to be reduced include, but are not limited to, draft animals, beasts of burden, animals useful in transportation (e.g., horses), racing animals (e.g., horses or greyhounds), meat animals, wool or fur-bearing animals, milk animals, working dogs, and companion animals, among others.

Administering the composition can be by any route, such as oral, intravenous, or intraarterial, among others. In one embodiment, administering may be by an oral route. In this embodiment, it may be desirable that the at least one polymethoxylated flavone be dissolved in a neutral or pleasant-tasting liquid, such as water, flavored water, milk, or fruit juice, among others. Additionally, the composition may be in tablet or capsule form and in this form the composition may be dissolvable in liquid. In other embodiments, the composition may be provided as a tablet or lozenge that dissolves when placed in the mouth of a user. In some embodiments, a composition according to any of the embodiments described herein can be provided in powder, tablet, capsule, gel, aerosol or liquid form.

In another embodiment, the present disclosure relates to a method, comprising administering, to a mammal suffering from or at risk of suffering from a sleep disorder, a composition comprising at least one polymethoxylated flavone.

A “sleep disorder,” as used herein, refers to a non-transient impairment of a mammal's ability to enter, exit, or remain in sleep at or during a desired time or duration. Though not to be bound by theory, one or more sleep disorders may arise or be exacerbated by a disruption in the mammal's circadian rhythm. In one embodiment, the sleep disorder may be selected from the group consisting of insomnia, hypersomnia, narcolepsy, delayed sleep phase disorder (DSPD), advanced sleep phase disorder (ASPD), non-24-hour sleep-wake disorder, irregular sleep wake rhythm, and shift work sleep disorder.

In the method of this embodiment, the composition, including the at least one polymethoxylated flavone and any additional components (such as one or more carriers, nicotinamide riboside, and/or pterostilbene), and also any formulations thereof, may be as described above. In one embodiment, the composition used in this method may comprise one or more ingredients expected to reduce one or more symptoms of a sleep disorder other than polymethoxylated flavones. For example, in one embodiment, the composition may further comprise melatonin, valerian, one or more valerenic acids, kava kava, one or more kavalactones, kavain, chamomile, apigenin, passionflower, lemon balm, skullcap, hops, lavender, L-tryptophan, St. John's wort, sour cherry, one or more phenolic acids, anthocyanin, and one or more cannabinoids.

Similarly, the administration of the composition alone or in combinations, including dosages, rates, durations, and routes, may also be as described above.

In yet another embodiment, the present disclosure relates to a method comprising administering, to a mammal suffering from aging, a composition comprising at least one polymethoxylated flavone.

“Aging,” as used herein, refers to an ongoing increase in senescence of a mammal. Though not to be bound by theory, aging may be exacerbated by a disruption in the mammal's circadian rhythm. Exemplary symptoms of aging include, but are not limited to, decline in muscle mass, decline in bone density, decline in immune system function, decline in memory, decline in cognitive function, increase in wrinkles, increase in liver spots, increase in gray and white hair, hair loss, and decline in overall vitality.

In the method of this embodiment, the composition, including the at least one polymethoxylated flavone and any additional components (such as one or more carriers, nicotinamide riboside, and/or pterostilbene), and also any formulations thereof, may be as described above. In one embodiment, the composition used in this method may comprise one or more ingredients expected to reduce one or more symptoms of aging other than polymethoxylated flavones. For example, in one embodiment, the composition may further comprise at least one of resveratrol, β-carotene, vitamin A, vitamin B6, vitamin B9, vitamin B12, vitamin C, vitamin E, one or more curcumins, turmeric, one or more green tea polyphenols, one or more catechins, epigallocatechin-3-gallate, grape seed extract, one or more carotenoids, lutein, zeaxanthin, cryptoxanthin, astaxanthin, canthaxanthin, lycopene, one or more xanthaphylls, one or more phytosterols, sitosterol, stigmasterol, campesterol, calcium, one or more omega-3 fatty acids, eicosapentaenoic acid, docosahexanoic acid, glucosamine, chondroitin, collagen, quercetin, dietary fiber, one or more probiotics, Lactobacillus, Bifidobacterium, one or more prebiotics, whey protein, potassium, zinc, coenzyme Q₁₀, ginkgo biloba, blueberry, cranberry, oregano, nectarine, acai, Rosa damascena, cocoa, green tea, olive oil, HSP-12.6, tannic acid, caffeic acid, rosmarinic acid, spermidine, or thioflavin T, among others.

Similarly, the administration of the composition, alone or in combinations, including dosages, rates, durations, and routes, may also be as described above.

In still another embodiment, the present disclosure relates to a method comprising administering, to a mammal suffering from or at risk of suffering from a mood disorder, a composition comprising at least one polymethoxylated flavone.

A “mood disorder,” as used herein, refers to a non-transient modification of a mammal's baseline emotional state. Examples of mood disorders include, but are not limited to mania, hypomania, unipolar depression, and bipolar disorder, among others. Though not to be bound by theory, a mood disorder may arise from or be exacerbated by a disruption in the mammal's circadian rhythm.

In the method of this embodiment, the composition, including the at least one polymethoxylated flavone and any additional components (such as one or more carriers, nicotinamide riboside, and/or pterostilbene), and also any formulations thereof, may be as described above. In one embodiment, the composition used in this method may comprise one or more ingredients expected to reduce one or more symptoms of mood disorder other than polymethoxylated flavones. For example, in one embodiment, the composition may further comprise at least one of St. John's wort, one or more omega-3 fatty acids, eicosapentaenoic acid, docosahexanoic acid, s-adenosyl-L-methionine (SAMe), ginkgo biloba, huperzine A, vitamin B6, vitamin B9, vitamin B12, vitamin D, caprylidene, or coconut oil, among others.

Similarly, the administration of the composition, alone or in combinations, including dosages, rates, durations, and routes, may also be as described above.

In additional embodiments, to a mammal suffering from or at risk of suffering from a cardiovascular disease, an immune disorder, a neurodegenerative disease, a cancer, or two or more thereof, a composition comprising at least one polymethoxylated flavone.

In the method of this embodiment, the composition, including the at least one polymethoxylated flavone and any additional components (such as one or more carriers, nicotinamide riboside, and/or pterostilbene), and also any formulations thereof, may be as described above. In one embodiment, the composition used in this method may comprise one or more ingredients expected to reduce one or more symptoms of the disease or disorder other than polymethoxylated flavones.

Similarly, the administration of the composition, alone or in combinations, including dosages, rates, durations, and routes, may also be as described above.

The following examples are included to demonstrate particular embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### EXAMPLES

### Example 1. Identification of Nobiletin as a Clock Modulator

To identify clock amplitude-enhancing small molecules (CEMs), an in-house compound collection with 5,300 small molecules was screened using heterozygous Clock^{Δ19/+} PER2::Luc reporter cells, which exhibit sustained reporter rhythms with a damped amplitude relative to wild type (WT) cells. Specifically, the chemical screen for circadian clock modulators was conducted at the Chemical Genomics Core facility at the University of Texas Health Science Center at Houston (UTHSC-H). The in-house chemical library screened consisted of compounds from the National Institutes of Health (NIH) Clinical Collection, National Cancer Institute collection, and Microsource Spectrum Collection. Screening was conducted largely on the basis of the protocol previously described (Chen et al., 2012). Briefly, immortalized fibroblast cells from Clock^{Δ19/+} heterozygous mice expressing the PER2::Luc bioluminescence reporter were plated into 96-well plates. Upon confluency, cells were incubated with 5 μM forskolin for 1-2 hr, followed by the addition of chemical compounds to the plates with robotic arms (Beckman), and then subjected to continuous monitoring over several days in a temperature-controlled EnVision microplate reader (Perkin Elmer). Data analysis was carried out by using the MultiCycle software (Actimetrics) for measurement of period, phase, and amplitude.

Further materials and methods were as follows:

Animals and Cell Lines.

Animal husbandry for all the studies except tissue explant experiments was carried out under IACUC guidelines and the procedures were conducted as described in an animal protocol approved by the University of Texas Health Science Center at Houston (UTHSC-H). Male wild-type (WT), Clock^{Δ19/Δ19}, db/db and db/db Clock^{Δ19/Δ19} mice, all on the C57BL/6J genetic background, were obtained as littermates from heterozygous breeding using Clock^{Δ19/+} (Antoch et al., 1997; King et al., 1997) and db/+ breeders obtained from the Takahashi lab and the Jackson Laboratory, #000697, respectively. Mice were group-housed (2-4/cage) in a standard animal facility under a 12 hr:12 hr light:dark cycle. Mice showing aggressive behaviors toward cage mates were removed. For circadian locomotor and metabolic chamber studies, mice were single-housed in a satellite facility approved by the Animal Welfare Committee of UTHSC-H. PER2::Luc reporter knock-in mice used for tissue explant experiments were maintained according to guidelines from IACUC at the University of Texas Southwestern Medical Center (UTSW). Adult mouse ear fibroblast and mouse embryonic fibroblast (MEF) cells were previously described (Chen et al., 2012).

High-Throughput Chemical Screen and Validation.

The chemical screen for circadian clock modulators was conducted at the Chemical Genomics Core facility at the UTHSC-H. The in-house chemical library screened consists of compounds from NIH Clinical Collection, NCI collection and Microsource Spectrum Collection. The screening was conducted largely based on the protocol previously described (Chen et al., 2012). Briefly, 15,000 immortalized fibroblast cells from Clock^{Δ19/+} heterozygous mice expressing the PER2::Luc bioluminescence reporter were plated into each well of 96-well plates, and incubated for 3-4 d to allow growth to confluency. Cells were then incubated with 5 μM forskolin for 1-2 h followed by the addition of chemical compounds to the plates with robotic arms (Beckman), and then subjected to continuous monitoring over several days in a temperature-controlled EnVision microplate reader (Perkin Elmer). Data analysis was carried out by using the MultiCycle software (Actimetrics) for measurement of period, phase, and amplitude. Among the compounds that showed greater than 2× SD effects on circadian amplitude and/or period, Nobiletin (NOB) was identified independently from two sub-libraries to significantly enhance circadian amplitude. NOB and a structurally related analog Naringin (NAR) were re-ordered from commercial sources including Sigma and GenDEPOT and dose response validation was conducted using PER2::LucSV reporter fibroblast cells which express stronger bioluminescence signals and thus allow precise measurements of circadian clock effects of compounds (Chen et al., 2012). Tissue explant experiments were conducted as described previously (Chen et al., 2012).

Circadian Locomotor Activity.

RC-fed WT, HFD-fed WT and Clock^{Δ19/Δ19} mice with NOB or Vehicle treatment were used for circadian locomotor activity experiments. Briefly, mice were first maintained for at least 2 weeks in a 12 hr:12 hr light:dark (LD) cycle, then released into the constant darkness, free-running condition. The mice were then maintained in constant darkness for another 2 weeks. Wheel-running data was downloaded as VitalView data files and analyzed with the ActiView and Actogram J program (Schmid et al., 2011; Zheng et al., 1999).

Mouse Treatment, Body Weight and Mass Composition Measurements.

For diet-induced obesity, male mice at 6 weeks of age were fed with HFD (D12492, Research Diets) until the end of the experimental protocol. The mice were treated with either vehicle (DMSO) or NOB (200 mg/kg body weight) via oral gavage every other day, in the time window of ZT8-10, throughout the experimental period. An every-other-day dosing regimen was chosen at the indicated level based on several reasons. First, previous in vivo studies have used similar overall amounts for mouse treatment (100-125 mg/kg/day) (Lee et al., 2013; Li et al., 2006). The oral gavage procedure was done in late afternoon (ZT8-10) prior to the start of their active phase, reasoning that this may coincide with the time-window adopted by previous studies. Second, daily dosing was not chosen to avoid entraining the experimental mice with the procedure per se as an artificial zeitgeber. Furthermore, pilot pharmacokinetic (PK) assays were done under a single-dose condition (Fig. S7). Consistent with a favorable PK profile, significant exposure in serum, brain and particularly liver was observed. Although NOB generally became undetectable 8 hr after single-dose administration in our study, other studies were able to measure NOB levels 24 hr after administration (Kumar et al., 2012; Singh et al., 2011). Therefore, it was reasoned that every-other day dosing helps avoid any incomplete daily clearance over the chronic experimental period (10 weeks).

Weekly body weight was monitored in different treatment mice for 10 weeks. Body mass composition was measured at the end of experiments using a minispec mqNMR spectrometer (Bruker Optics, Texas) (Garcia et al., 2013). A control group of mice were fed with regular chow diet (Purina 5001) in parallel with the two treatment groups in HFD. For db/db and db/db Clock^{Δ19/Δ19} mice, male mice at 6-8 weeks of age were group-housed (2-3/cage) and maintained on regular chow diets. Mice were subjected to oral gavage with either DMSO or NOB as described above.

Pharmacokinetic Study in Mice.

NOB was administered orally at ZT8 at a dose of 200 mg/kg in 0.25% sodium Carboxy Methyl Cellulose (CMC) suspension. Three mice per time point were sacrificed at 0.0, 1.0, 2.0, 4.0, 8.0 and 24.0 hr after oral gavage. NOB in plasma, brain and liver was determined by LC-MS/MS (API 4000: EQ-RS-MS-006).

Energy Expenditure and Food Intake Measurements.

Energy expenditure was examined by measuring oxygen consumption with indirect calorimetry as described (Chutkow et al., 2010; Daniels et al., 2010). After 8 weeks of treatments described above, mice from each group were placed at room temperature (22° C.-24° C.) in the chambers of a Comprehensive Lab Animal Monitoring System (CLAMS, Columbus Instruments, Columbus, Ohio). After mice adapted to the metabolic chamber, volume of O2 consumption and CO2 production was continuously recorded over a 24-hr period. Average O2 consumption was calculated and compared between different treatments. Food and water were provided ad libitum. To measure food intake, food pellets were weighted every three hours over a 24-hr period in mice treated as above. The daily food intake was calculated from averaged food intake of 3 independent experiments.

Serum and Liver Lipid Assays.

Serum samples were obtained at ZT2 from treated mice as previously described (Jeong et al., 2015). Hepatic triglyceride and cholesterol were extracted as previously described (Liu et al., 2012). The triglyceride and cholesterol levels in liver and serum were assessed by Serum Triglyceride Determination Kit (Sigma) and Cholesterol Assay Kit (Cayman), respectively. The assay plates were read by a TECAN M200 instrument (Tecan) following the manufacturer's instructions.

Glucose Tolerance and Insulin Tolerance Tests (GTT and ITT).

Glucose tolerance test (GTT) and insulin tolerance test (ITT) were performed largely as described (He et al., 2015; Jeong et al., 2015). Briefly, after overnight and 5-hr fasting, GTT and ITT were conducted at ZT2 and ZT8 respectively. Glucose levels were measured from tail blood before and 15, 30, 60, or 120 min after injection of either 1 g/kg glucose or 0.75 U/kg insulin (Sigma) at ZT2 and ZT8 respectively by using the ONETOUCH UltraMini blood glucose monitoring system (LifeScan). Serum insulin levels were measured with the Rat/Mouse Insulin Elisa kit (Millipore) according to the manufacturer's instructions. The plasma samples were collected at ZT2 as described above for lipid assays.

Histological Analysis of Liver and Adipose Tissues.

For microscopic analysis of lipid accumulation in liver, tissue samples were collected and immediately embedded in Tissue-Tek OCT cryostat molds (Leica) and then frozen at −80° C. Tissue sections were stained in 0.5% Oil Red O and counterstained with Mayer's hematoxylin for 1 min. In addition, liver, brown fat and white fat tissues were embedded in paraffin and stained with Hematoxylin and Eosin (H&E). Microscopic images were obtained on an Olympus BX60 microscope.

Real-Time qPCR and Western Blot Analyses.

For qPCR analysis, cells were split into 6-well plates at an initial density of 3×105 cells and incubated for 2-4 days before synchronization (5 μM Fsk or 100 nM Dex) followed by compound treatment. RNA samples were prepared by using PureXtract RNAsol for cDNA synthesis and real-time PCR (GenDEPOT) was performed with a MaxPro3000 Thermocycler (Agilent). qPCR primers used are listed in the table shown in Example 4. Whole cell lysates from cells similarly grown and treated in 60 mm dishes and tissue extracts were prepared as described (Yoo et al., 2013) and subjected to Western blot analysis (GenDEPOT). Antibodies for REV-ERBα (Pierce and Cell Signaling), PER1 (Lee et al., 2001) and other clock proteins (Yoo et al., 2013) were used.

Microarray Analysis.

Total RNAs prepared from liver tissues from RC-fed, Vehicle-treated and HFD-fed, Vehicle or NOB-treated WT mice for 10 weeks were reverse transcribed into cDNAs, which were then biotin-UTP labeled and hybridized to the Illumina mouse WG-6v2.0 Expression BeadChip. Genes with statistically significant fold change differences was clustered using centered correlation (Cluster 3.0) and then visualized as a heat map on Tree View. Moreover, genes with statistically significant differences derived from microarray analyses were imported into the Ingenuity Pathway Analysis (IPA, http://www.ingenuity.com). In IPA, differentially expressed genes are mapped to genetic networks in the Ingenuity Knowledge Database to generate a set of network and then ranked by score. Heatmaps were generated by using Cluster 3.0 program. ChIP-seq data analysis was conducted as previously described (Koike et al., 2012). The data discussed in this publication have been deposited in NCBI's Gene Expression Omnibus (Edgar et al., 2002) and are accessible through GEO Series accession number GSE78848.

Plasmids.

Plasmids containing the retinoic acid-related orphan receptor response element (RORE) from mouse Bmal1 promoter, pcDNA3.1B-G4DBD-RORαLBD and pcDNA3.1BG4DBD-RORγLBD were constructed in our lab. Specifically, the 1960 bp sequences (−1830/+130) of the mouse Bmal1 promoter harboring either WT (AAAGTAGGTCA (SEQ ID NO:1) and AAAGTAGGTTA (SEQ ID NO:2)) or mutant (AAAGTACACGA (SEQ ID NO:3)) RORE were PCR amplified from mouse genomic DNA and cloned into pGL3-promoter luciferase vector. For expression of RORα or RORγ-GAL4 protein, mouse RORα-LBD (aa 261-523) or RORγ-LBD (aa 250-516) were PCR amplified from mouse genomic DNA and cloned into a GAL4-DBD-containing vector, pcDNA3.1B.

One-Hybrid Reporter Assays.

For mammalian one-hybrid assays, HEK293T cells were cotransfected with pcDNA3.1B-G4DBD-RORα/γLBD, pGL4.31 and TK promoter Renilla luciferase construct (tK.pRL). To investigate the regulation of the Bmal1 reporter, Hepa1-6 cells were cotransfected with Bmal1-WT or mutant RORE reporter plasmids, RORα, RORγ or Rev-erbα expression construct along with tK.pRL. Mouse and human siRNA targeting RORα and RORγ were purchased from Santa Cruz. Transfection was performed by Lipfectamine 2000 reagent (Invitrogen) Twenty-four hours after transfection, the cells were treated with vehicle or NOB. Lysates were collected 24 h after treatment, and firefly and Renilla luciferase activities were measured by using a Dual-Luciferase Reporter System (Promega). Regardless of the nature of the ligand (agonist or inverse agonist), ligand interaction with these chimeric receptors has been shown to reduce transcriptional activity of these chimeric receptors (Wang et al., 2010a; Wang et al., 2010b).

Radioligand Receptor Binding Assays.

Previously described protocols with minor modifications (Kumar et al., 2010; Wang et al., 2010b). For saturation binding experiments, 100 ng RORα-LBD or 200 ng RORγ-LBD was incubated with 25-[3H]-hydroxycholesterol (OHC) in assay buffer [50 mM HEPES, pH. 7.4, 0.05% bovine serum albumin (BSA), 150 mM NaCl and 5 mM MgCl2]. Ligand binding was determined by filter binding assays to calculate the Kd value. For competitive binding assay, 100 ng RORα-LBD or 200 ng RORγ-LBD was incubated with various concentrations of Nobiletin, Naringin or Naringenin in the presence of 4.5 nM 25-[3H]—OHC. Ki was determined using the Cheng-Prusoff equation.

RNA-Mediated Interference.

Hepa1-6 cells on the 24-well plate were transfected using control siRNA and siRNA against mouse RORα and RORγ (Santa Cruz). Twenty four hours after transfection, cells were treated with DMSO or NOB (3 μM). After 12 hr treatment, cells were harvested and total RNA was insolated. Real-time qPCR was performed to analyze the mRNA expression of mouse Rora and Rorc with a MaxPro3000 Thermocycler (Agilent).

Statistical Analysis.

Data are presented as mean±SEM. Statistical significance was determined by one-way or two-way ANOVA with Turkey's and Dunnett's tests for multiple group comparisons. P<0.05 was considered to be statistically significant.

In all examples, data are presented as mean±SEM. Statistical significance was determined by one-way or two-way ANOVA with Turkey or Dunnett tests for multiple-group comparisons. A p value <0.05 was considered to indicate statistical significance.

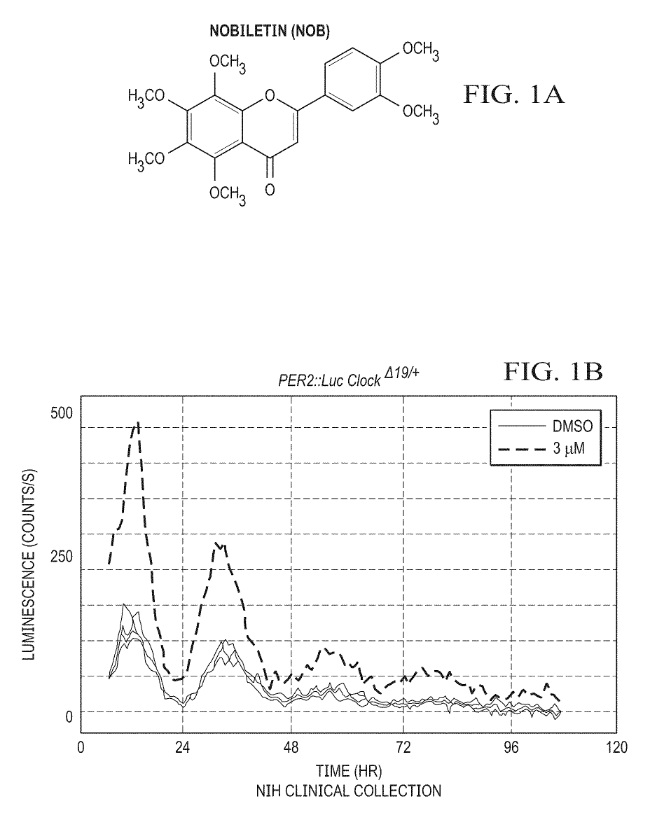

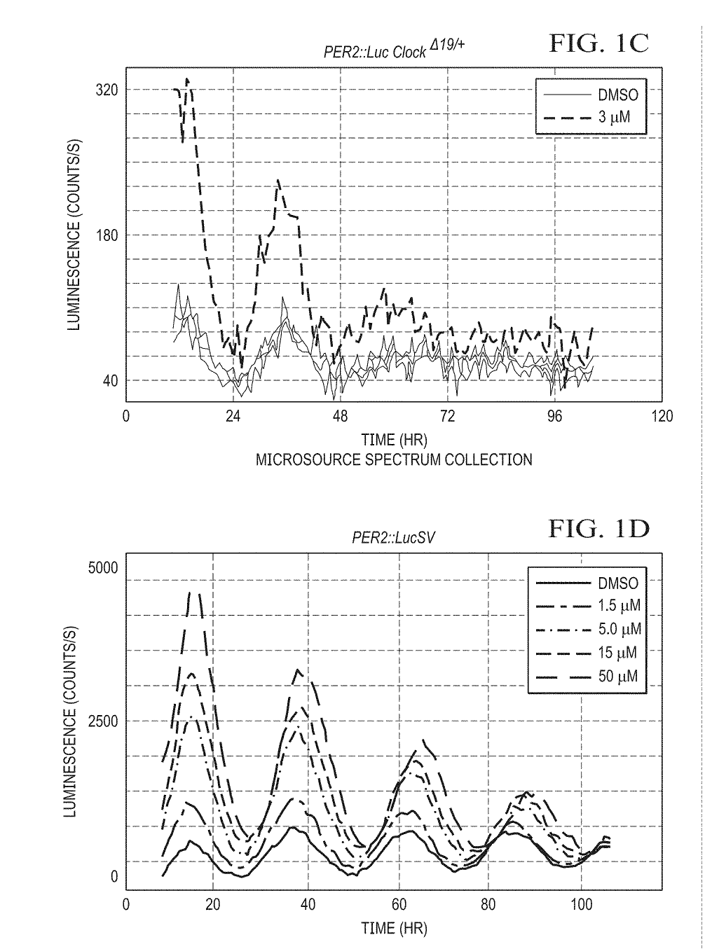

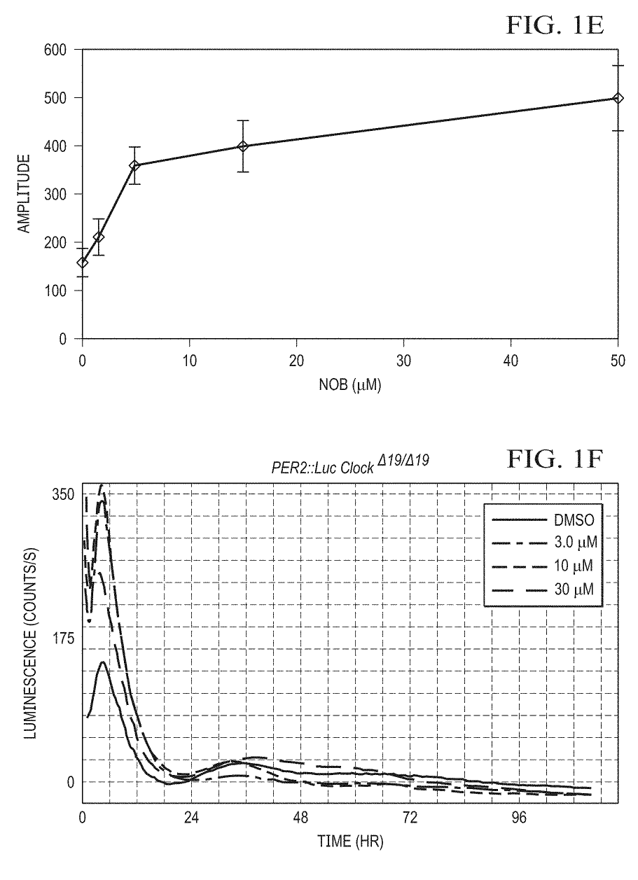

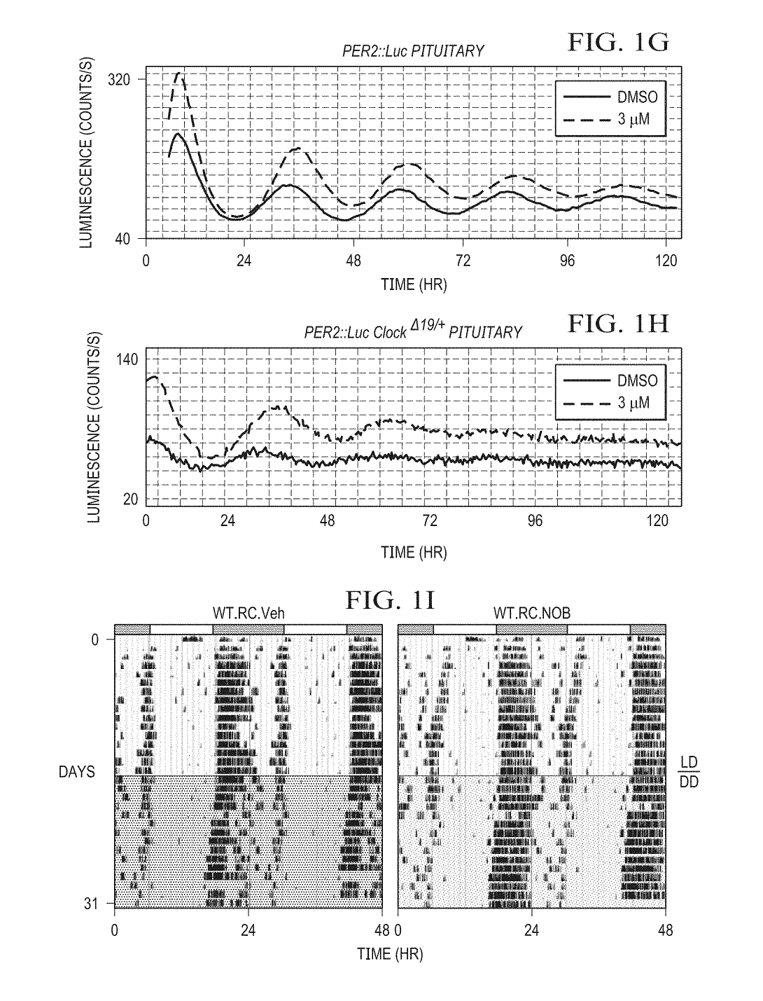

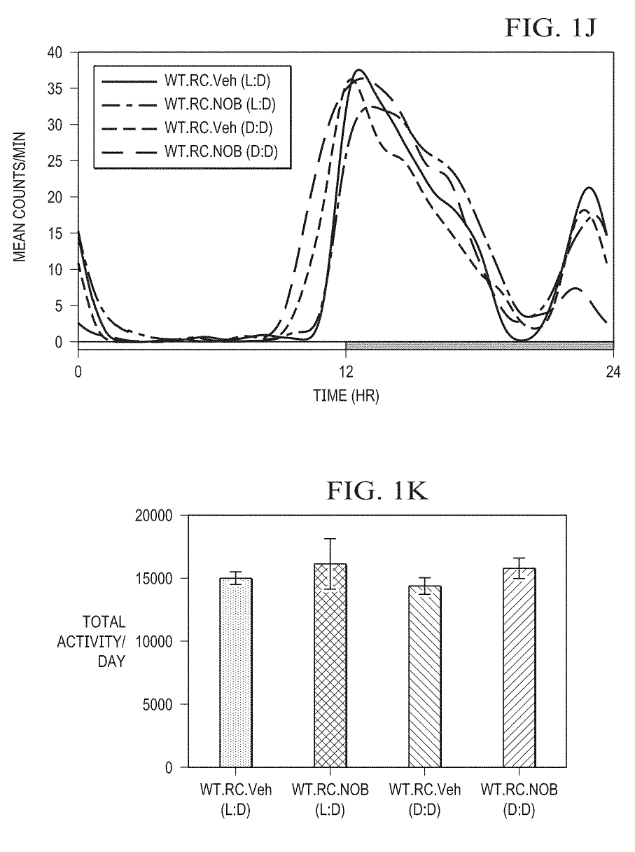

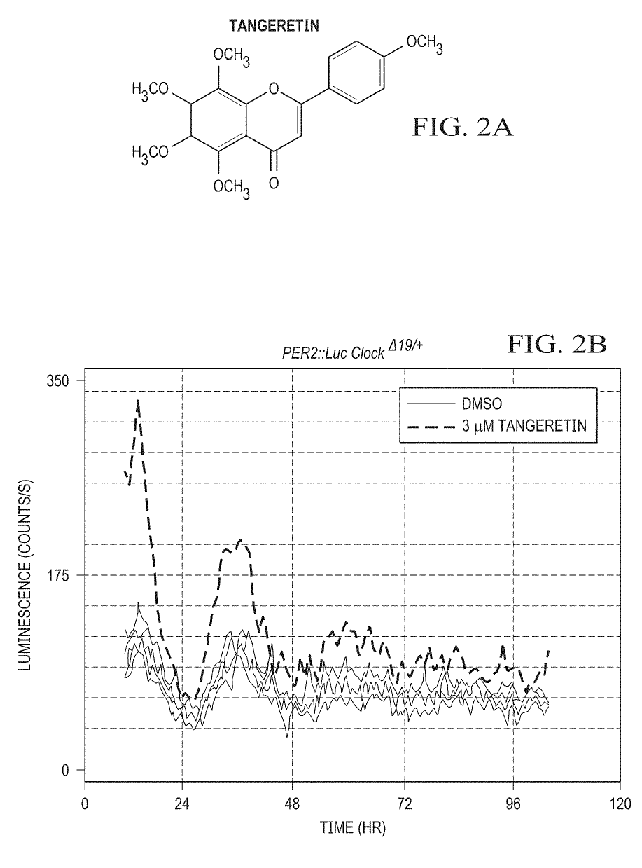

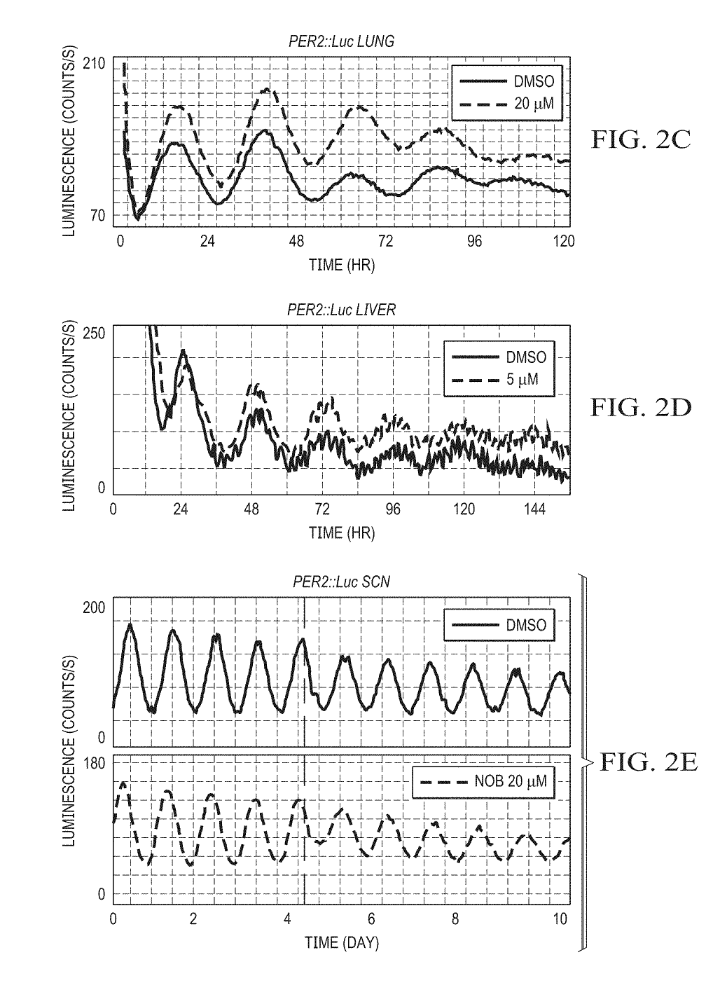

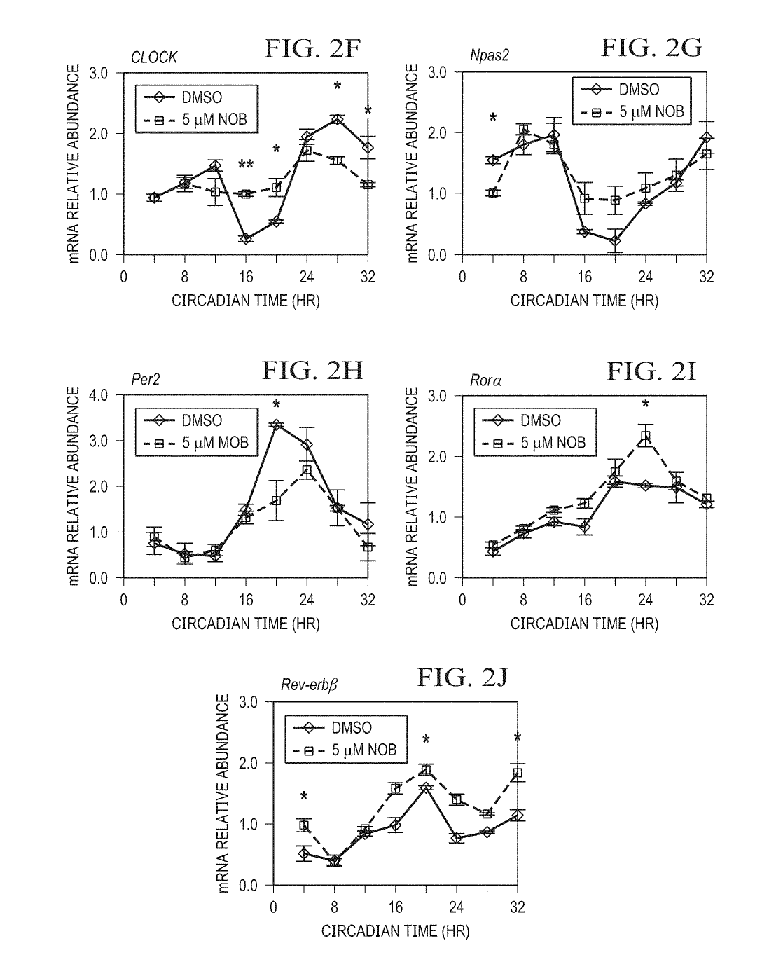

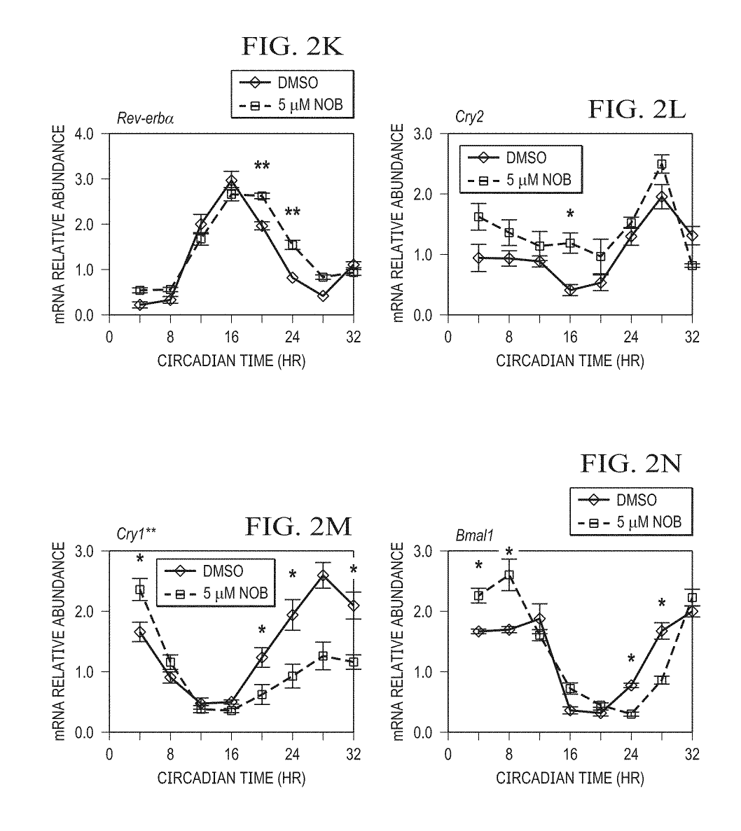

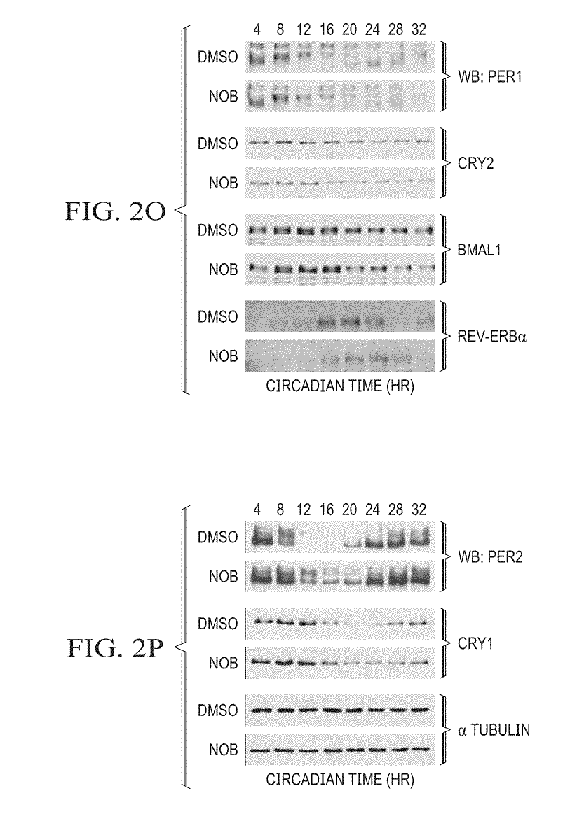

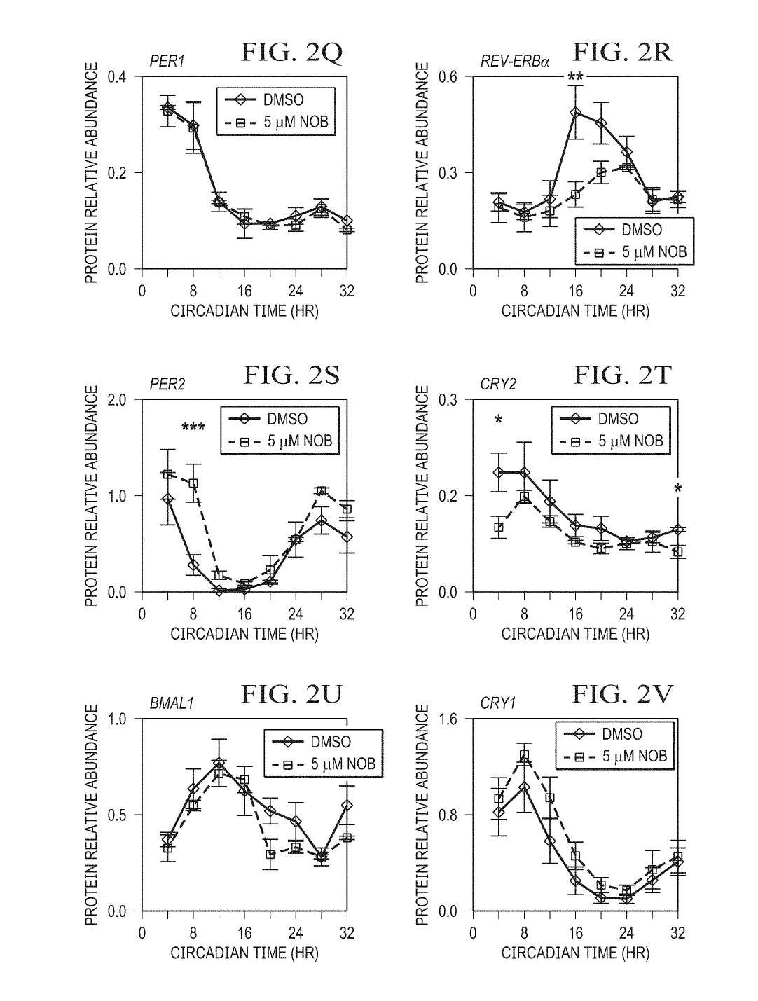

A naturally occurring polymethoxylated flavonoid enriched in citrus peels, nobiletin, was found independently from two sub-libraries to enhance the reporter rhythm of the Clock^{Δ19/+} cells (FIGS. 1A, 1B, and 1C). Tangeretin, a close analog of nobiletin, similarly enhanced the PER2::Luc reporter rhythm in Clock^{Δ19/+} cells (FIGS. 2A and 2B). Nobiletin robustly enhanced the amplitude of PER2::LucSV reporter rhythm (Chen et al., 2012) and also lengthened the period in a dose-dependent manner, with an estimated half maximal effective concentration of <5.0 mM (FIG. 1C-1E). Similar to previously reported CEMs (Chen et al., 2012, 2013), nobiletin was ineffective in restoring the rhythm in clock-disrupted homozygous Clock^{Δ19/Δ19} reporter cells (FIG. 1F). Importantly, nobiletin enhanced PER2::Luc reporter rhythms in peripheral tissue explants from both Clock^{Δ19/+} and WT reporter knockin mice (FIGS. 1G, 1H, 2C, and 2D), but not in the SCN, which is resistant to external perturbation due to robust inter-neuronal coupling (FIG. 2E) (Buhr et al., 2010; Chen et al., 2012; Liu et al., 2007). In accordance with the resistance of the SCN to nobiletin manipulation, normal wheel-running activity and periodicity in WT C57/6J mice treated with nobiletin (FIG. 1I-1K) was observed.

Nobiletin has shown a wide variety of beneficial effects (Ben-Aziz, 1967; Cui et al., 2010; Mulvihill et al., 2011; Nagase et al., 2005; Walle, 2007). However, its role as a modulator of the circadian clock was previously unknown. Whereas Pert transcript levels were moderately altered and reduced at CT20 by nobiletin in PER2::LucSV cells (FIG. 2F), PER2 proteins were found to accumulate to greater levels (FIGS. 2O-2V), consistent with the elevated bioluminescence and suggesting a post-transcriptional mechanism for PER2 enrichment. Expression of other core clock genes was also altered by nobiletin (FIGS. 2F-2V). In particular, CRY1, the heterodimeric partner of PERs in the negative arm of the oscillator, showed a slight trend of greater protein abundance despite markedly reduced transcript level (FIGS. 2F-2P). The concomitant enrichment of PER2 and CRY1 is consistent with the idea that PER2 and CRY1 proteins stabilize each other (Yagita et al., 2002) and that an improved stoichiometric ratio of the negative arm can lead to enhanced overall circadian amplitude in mouse fibroblast cells (Lee et al., 2011).

### Example 2. Robust Clock-Dependent Metabolic Protection by Nobiletin in Diet-Induced Obese (DIO) Mice

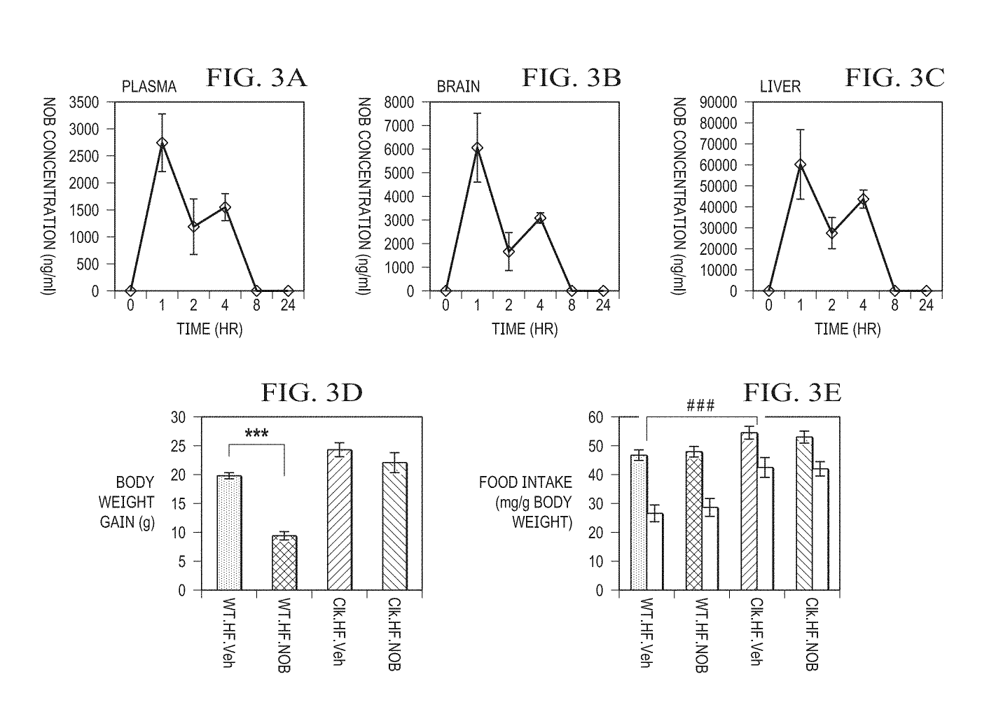

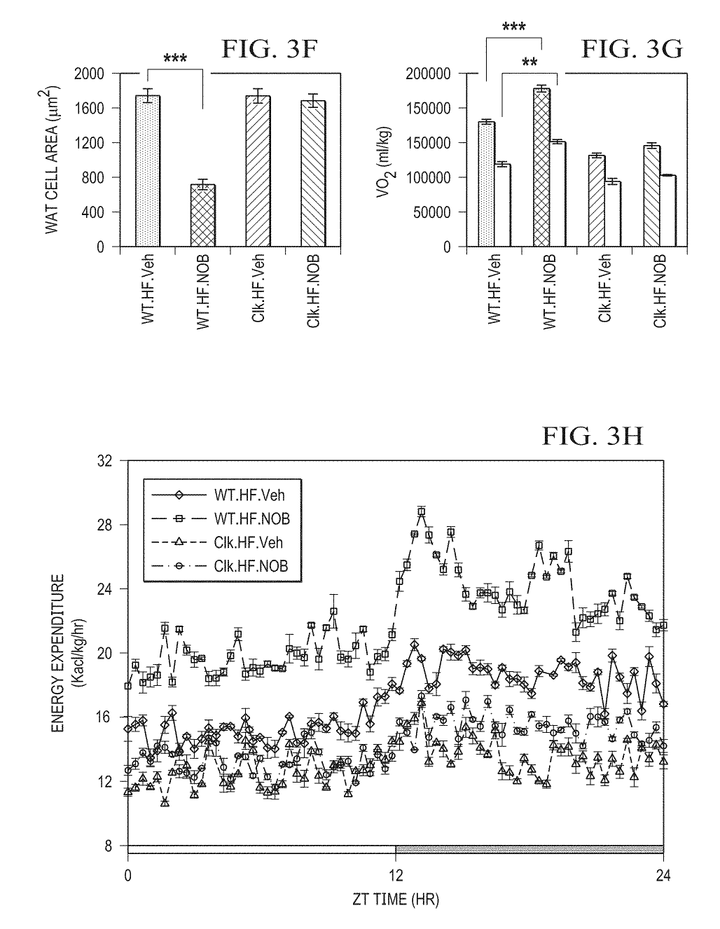

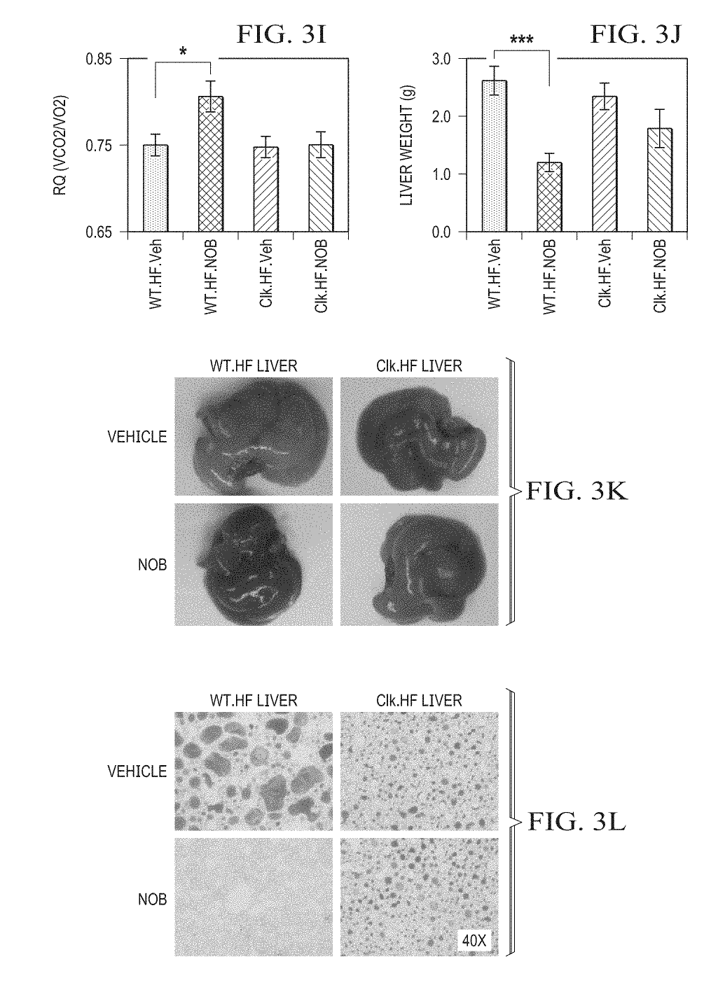

Recent studies suggested a protective role of nobiletin against metabolic syndrome (Kurowska and Manthey, 2004; Lee et al., 2010, 2013; Mulvihill et al., 2011; Roza et al., 2007). In accordance, pharmacokinetic studies revealed significant brain and systemic exposure of nobiletin (FIG. 3A-3C). To address whether metabolic protection by nobiletin depends on clock function, a diet-induced obese (DIO) mouse model using both WT and clock-disrupted Clock^{Δ19/Δ19} mice was chosen.

Animal husbandry for all the studies except tissue explant experiments was carried out under Institutional Animal Care and Use Committee (IACUC) guidelines, and the procedures were conducted as described in an animal protocol approved by the University of Texas Health Science Center at Houston (UTHSC-H). PER2::Luc reporter knockin mice used for tissue explant experiments were maintained according to guidelines from the IACUC at the University of Texas Southwestern Medical Center. Adult mouse ear fibroblast and mouse embryonic fibroblast cells were previously described (Chen et al., 2012).

For diet-induced obesity, male mice at 6 weeks of age were fed with HFD (D12492; Research Diets) until the end of the experimental protocol. The mice were treated with either vehicle (DMSO) or nobiletin (200 mg/kg body weight) via oral gavage every other day, in the time window of ZT8-ZT10, throughout the experimental period. The every-other-day dosing regimen was chosen at the indicated level for several reasons described elsewhere herein. Metabolic assays and energy expenditure analyses were conducted as previously described (Daniels et al., 2010; Garcia et al., 2013; He et al., 2015; Jeong et al., 2015).

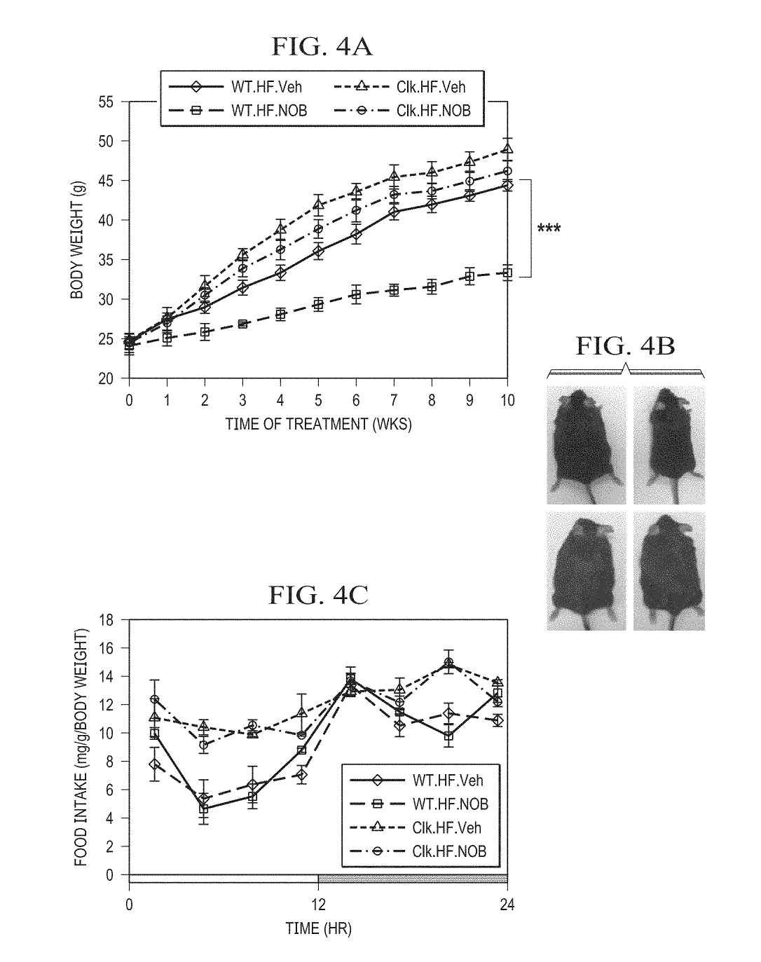

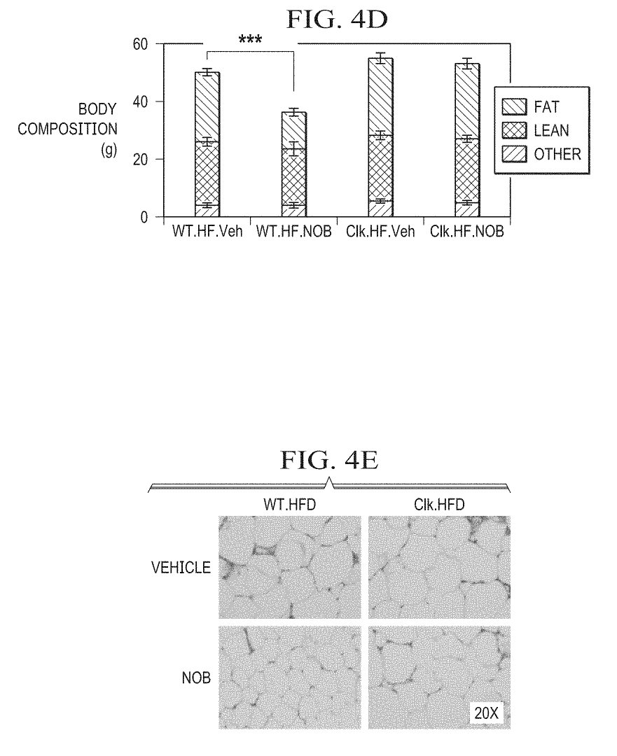

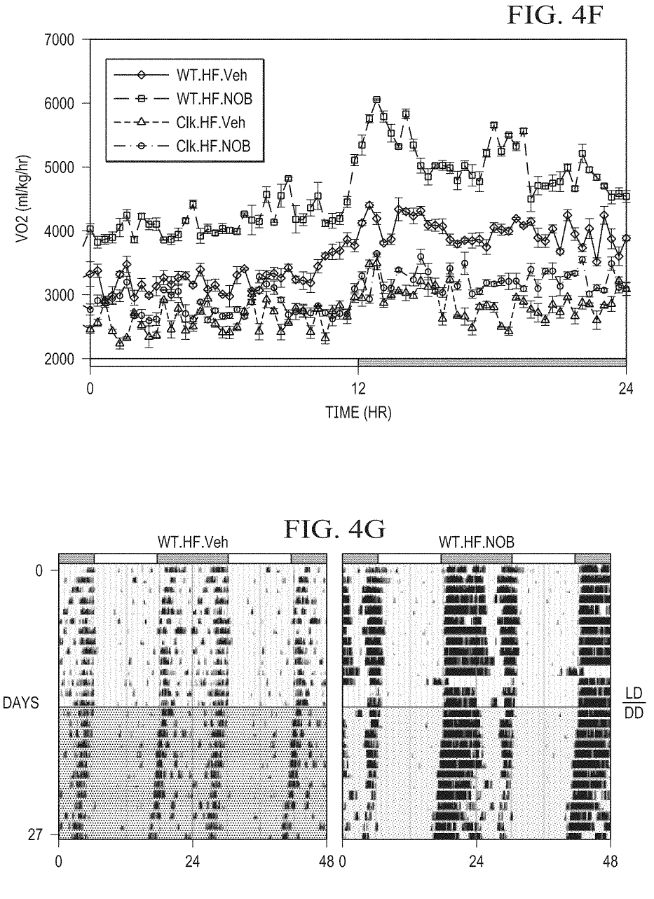

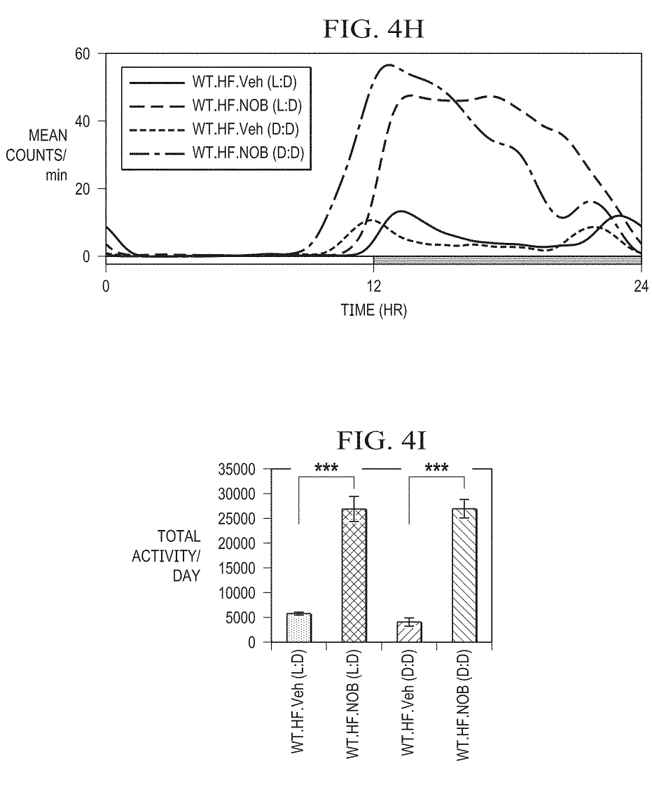

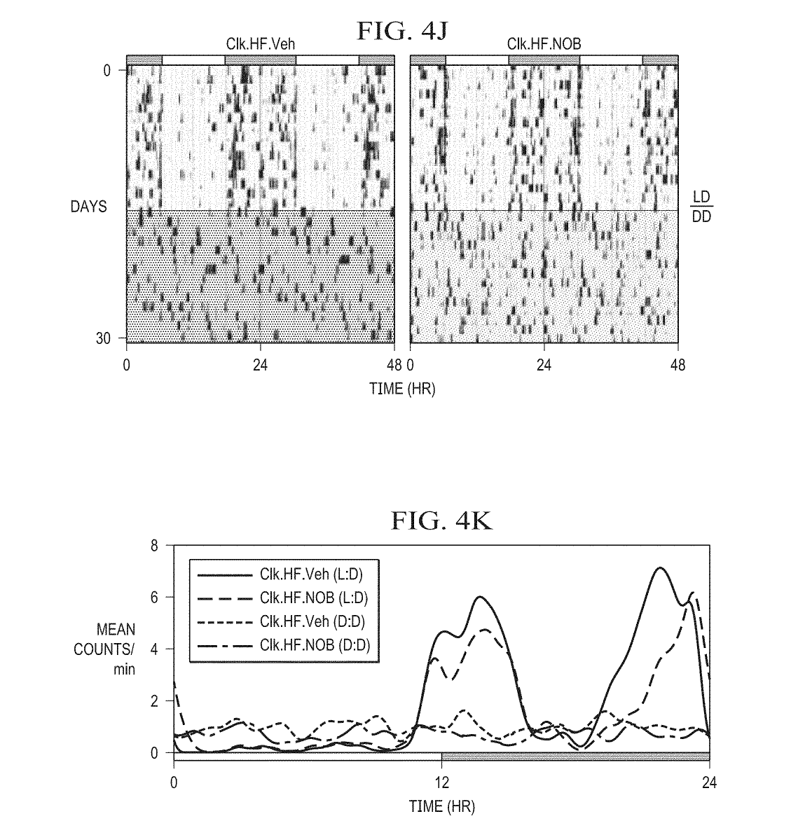

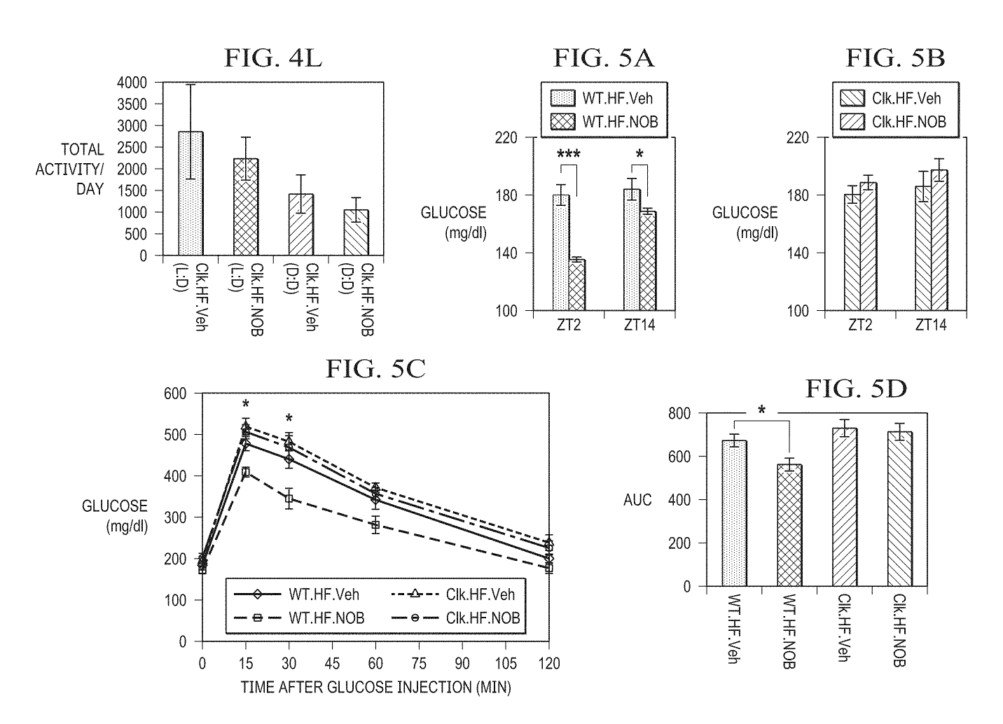

In WT C57BL/6J mice fed with HFD, 10-week nobiletin treatment significantly attenuated body weight gain relative to the vehicle control (FIGS. 4A, 4B, and 3D). Food intake was not significantly altered by nobiletin relative to the vehicle control (FIGS. 4C and 3E). Body mass composition analysis revealed that the body weight loss was primarily attributable to reduction in fat mass (FIG. 4D) and white adipose cell size (FIGS. 4E and 3F). Remarkably, nobiletin treatment only led to very modest reduction in body weight gain in HFD-fed Clock^{Δ19/Δ19} mutant mice (FIGS. 4A, 4B, and 3D), with the fat mass and adipocyte cell size essentially unchanged by nobiletin treatment (FIGS. 4D, 4E, and 3F). Although mutant mice consumed more food during the light phase than WT mice (Turek et al., 2005), nobiletin did not change food intake in the mutant mice (FIGS. 4C and 3E). Indicating elevated energy expenditure, nobiletin-treated WT mice exhibited greatly elevated oxygen consumption compared with the controls throughout the circadian cycle, with the largest increase found in early dark phase (FIGS. 4F and 3G). Respiratory quotient was also increased in WT mice treated with nobiletin (FIG. 3H), consistent with a switch from lipid-biased metabolism to a more balanced contribution from all major macronutrients. In contrast, Clock^{Δ19/Δ19} mice showed no increase in energy expenditure or respiratory quotient after nobiletin treatment (FIGS. 4F and 3H). Consistent with its effect on body weight and energy expenditure, nobiletin greatly increased wheel-running activity levels in HFD-fed WT mice relative to control treatment (FIG. 4G); in contrast, no significant difference in activity levels was detected between the treatments for Clock^{Δ19/Δ19} mice (FIG. 4H-4J).

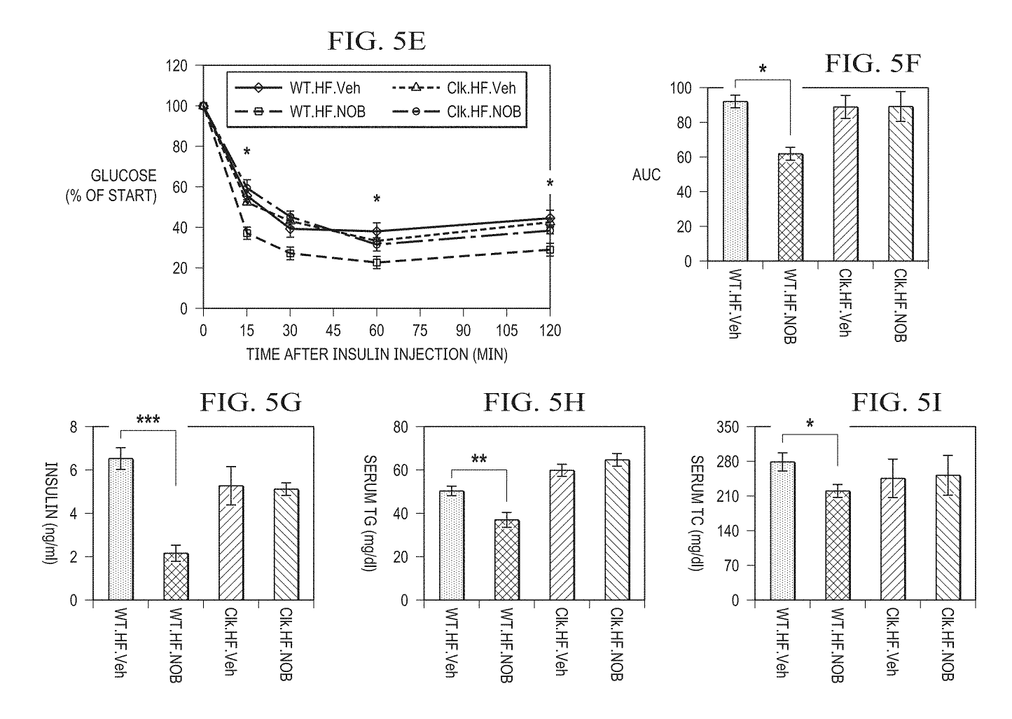

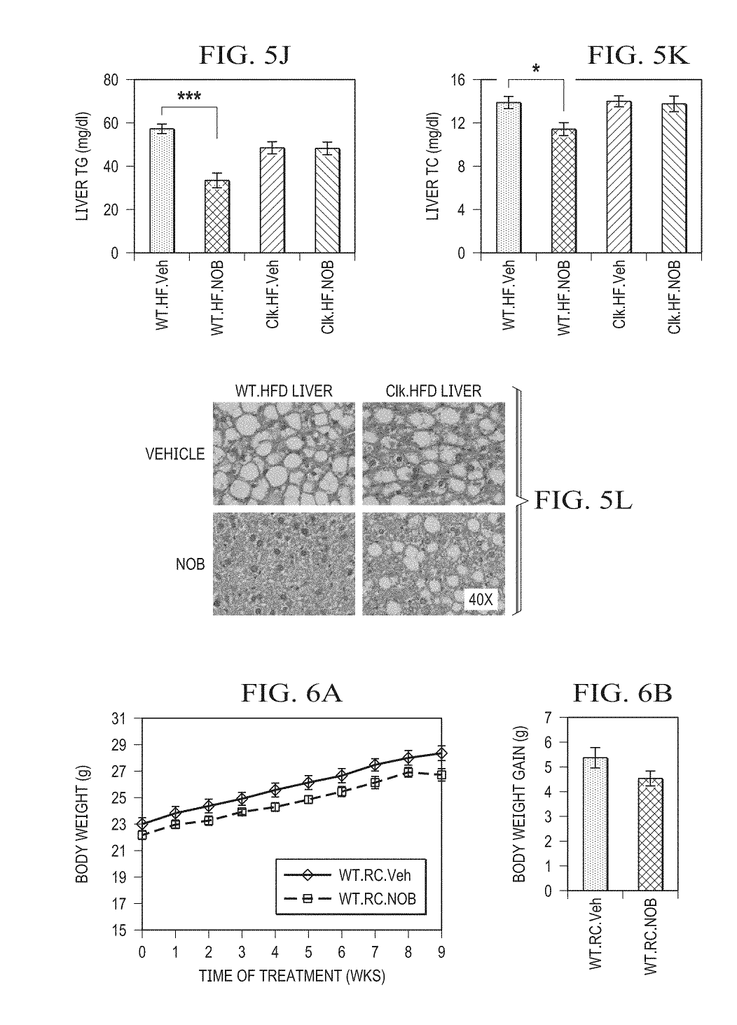

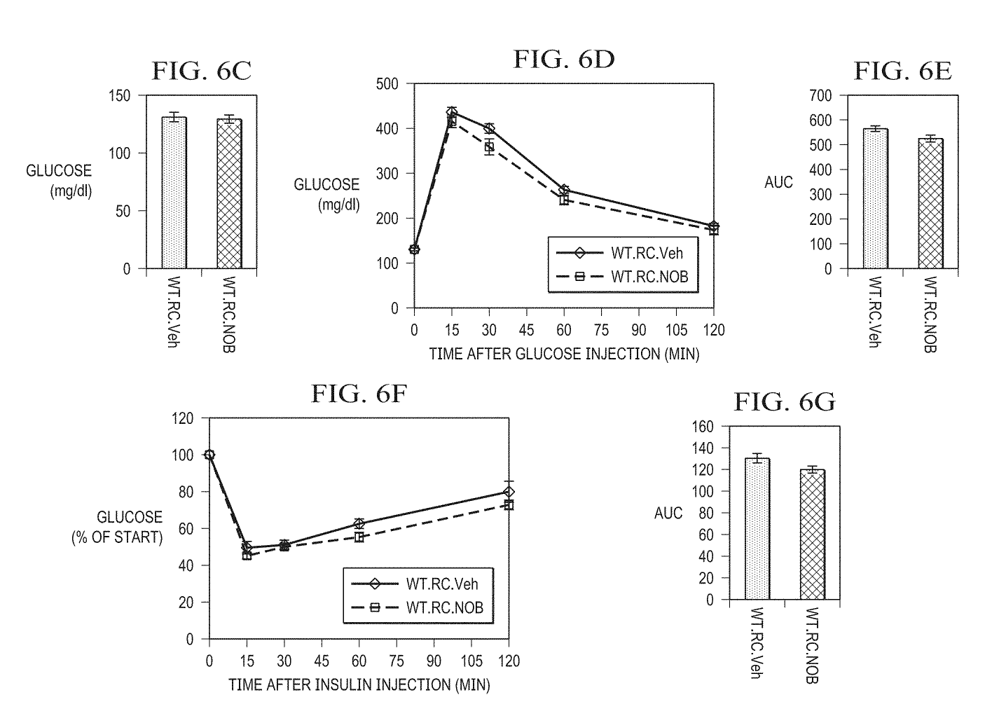

Nobiletin also improved glucose and lipid homeostasis in WT but not Clock^{Δ19/Δ19} mice. Nobiletin lowered fasting glucose levels in WT mice (FIG. 5A-5B) and significantly improved glucose tolerance and insulin sensitivity (FIGS. 5C-5F). Interestingly, blood insulin levels were strongly reduced in nobiletin-treated WT mice relative to vehicle controls (FIG. 5G). Total triglyceride (TG) and total cholesterol (TC) levels in WT serum and liver were also significantly diminished by nobiletin (FIGS. 5H-5K). Hematoxylin and eosin (H&E) and Oil Red O staining revealed that nobiletin strongly improved liver steatosis and essentially abolished lipid droplet formation in DIO WT liver (FIGS. 5L and 3J-3K). In contrast, nobiletin did not significantly improve glucose and lipid homeostasis in Clock^{Δ19/Δ19} mice (FIG. 5), and its beneficial effect on Clock^{Δ19/Δ19} liver steatosis was markedly attenuated compared with that in WT mice (FIGS. 5L and 3J-3K). In contrast to HFD, regular chow (RC) feeding did not lead to metabolic disorders, and nobiletin treatment did not show significantly beneficial effects on metabolic homeostasis (FIG. 6A-6G). Together, these results demonstrate a Clock-dependent efficacy of nobiletin against metabolic syndrome.

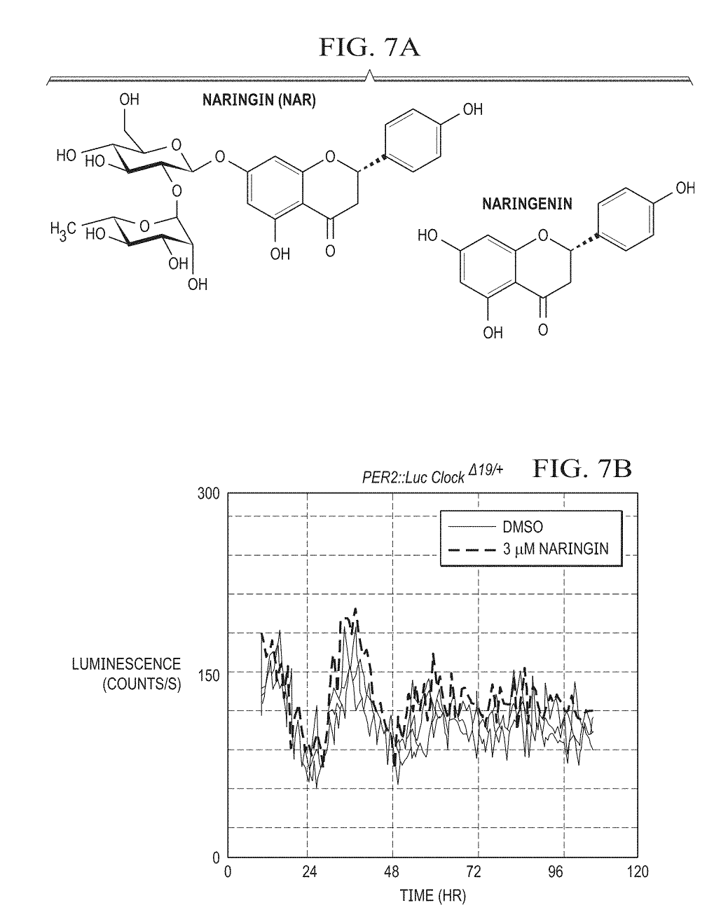

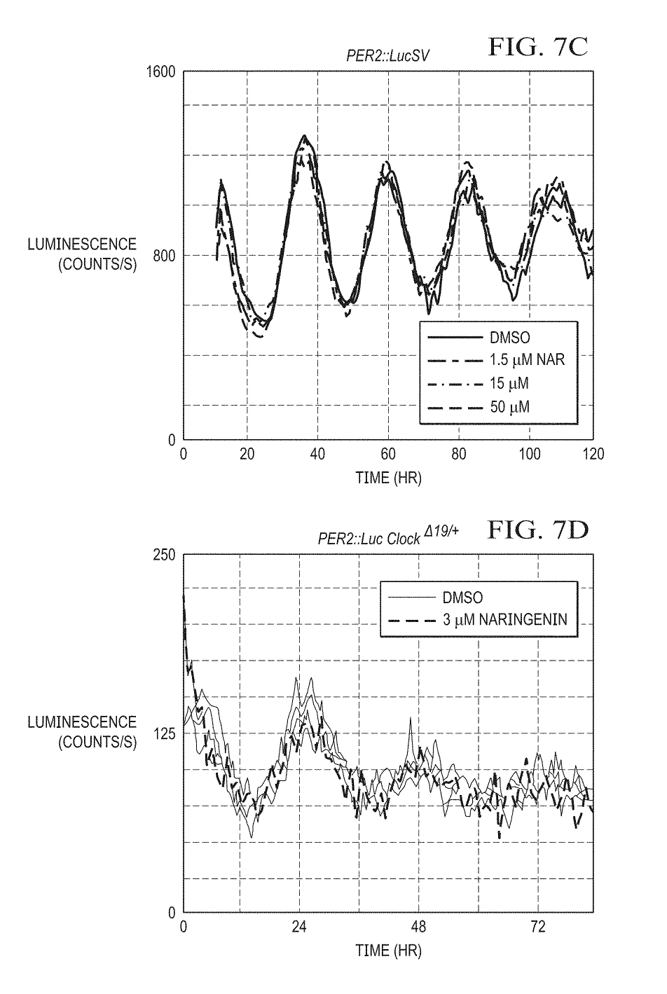

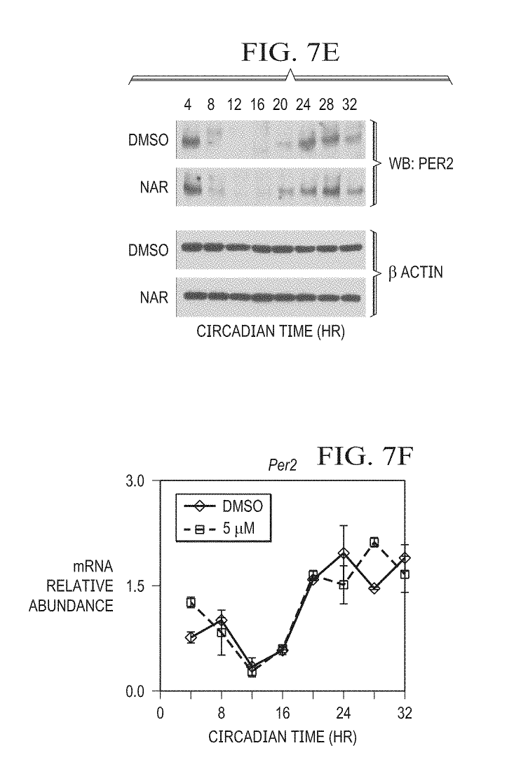

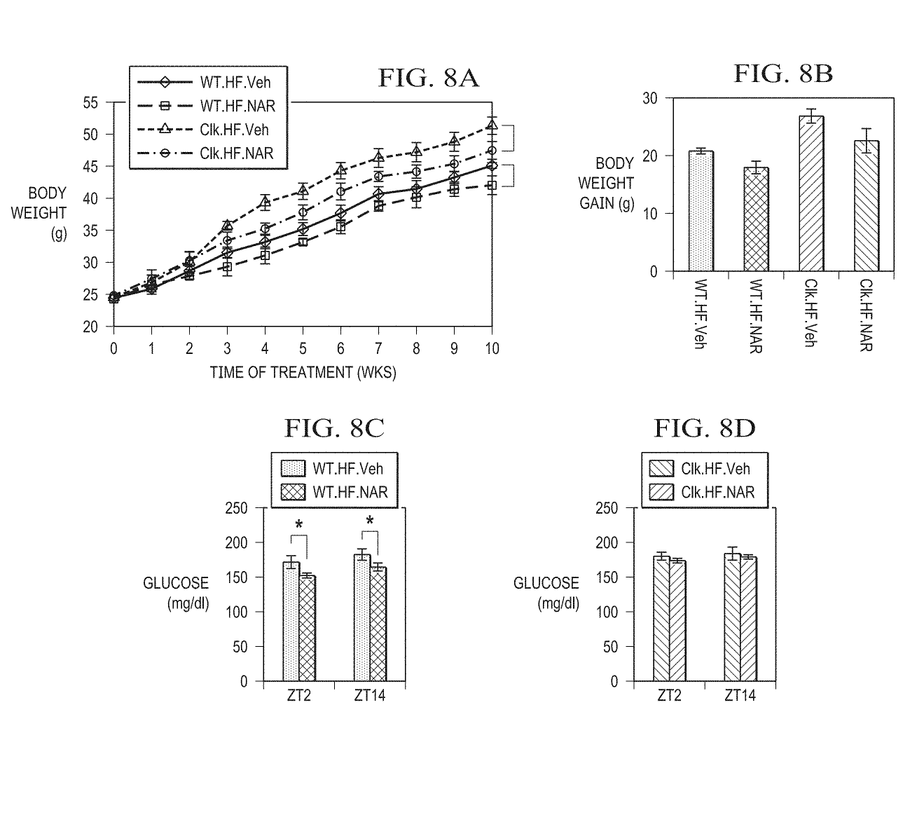

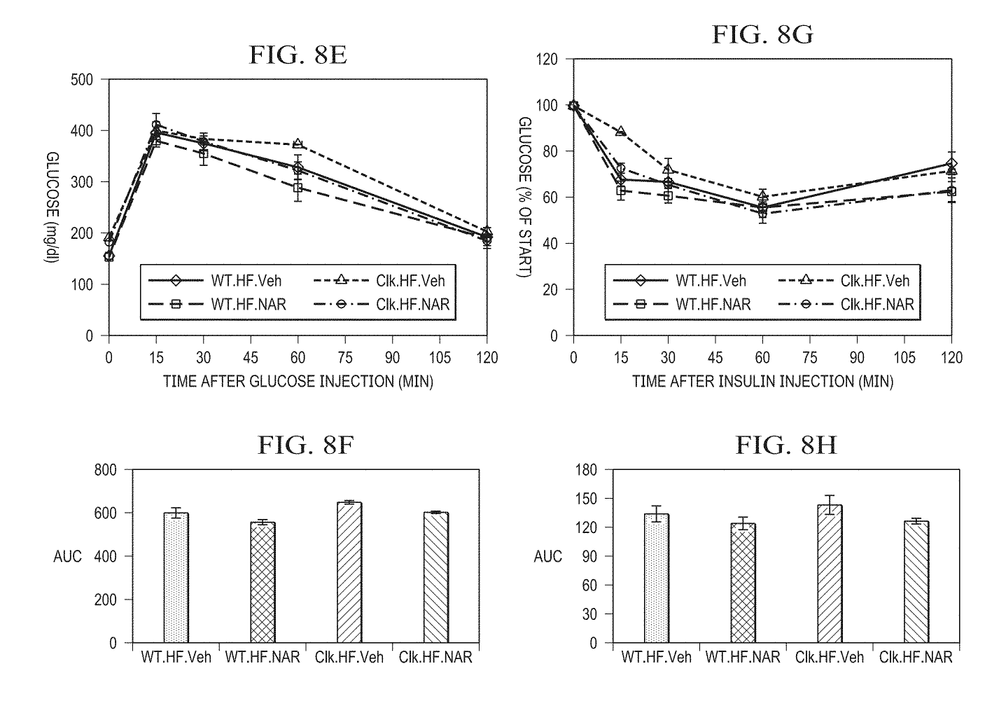

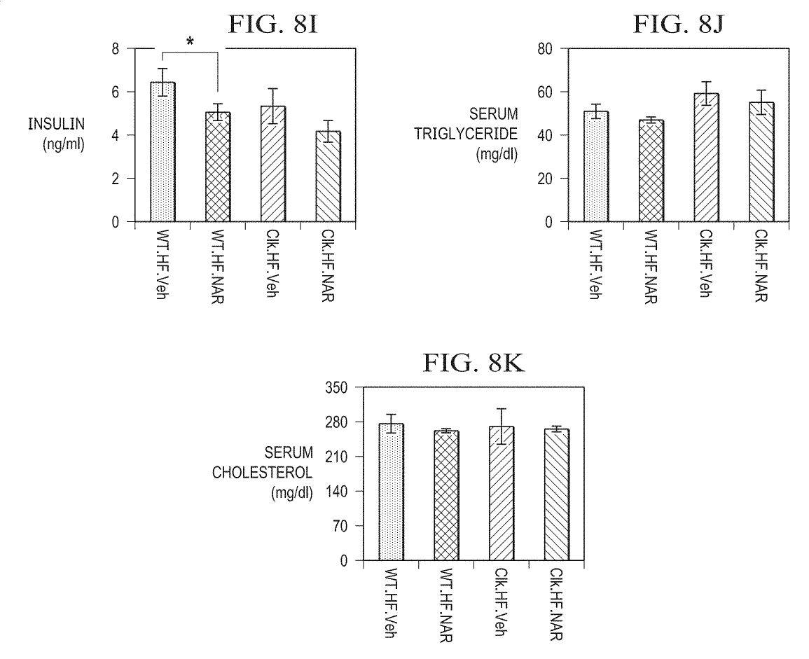

Non-methoxylated flavanones such as naringin and its aglycone derivative naringenin are also naturally occurring flavonoids (FIG. 7A) (Assini et al., 2013), yet they failed to enhance cellular circadian rhythms in our primary screen (FIGS. 7B-7F). Consistent with previous studies (Mulvihill et al., 2009, 2011), compared with nobiletin, naringin showed significantly attenuated effects on body weight gain and lipid and glucose homeostasis (FIG. 8A-8K). Importantly, the modest effects from naringin treatment were largely indistinguishable between WT or Clock^{Δ19/Δ19} C57BL/6J mice.

### Example 3. Clock-Dependent Metabolic Protection by Nobiletin in Db/Db Diabetic Mice

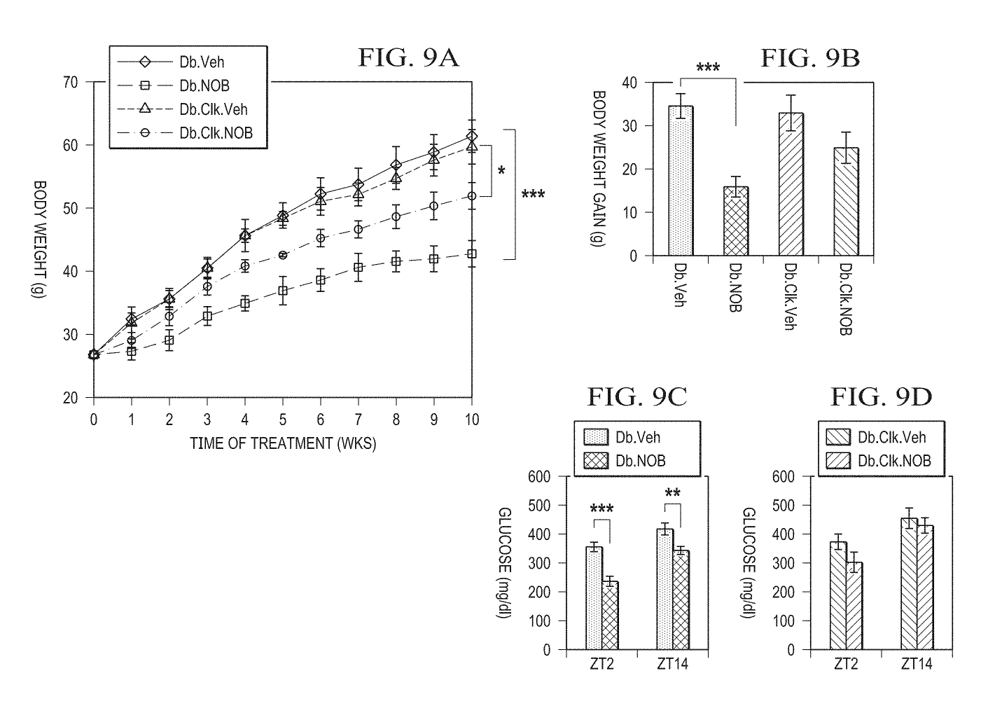

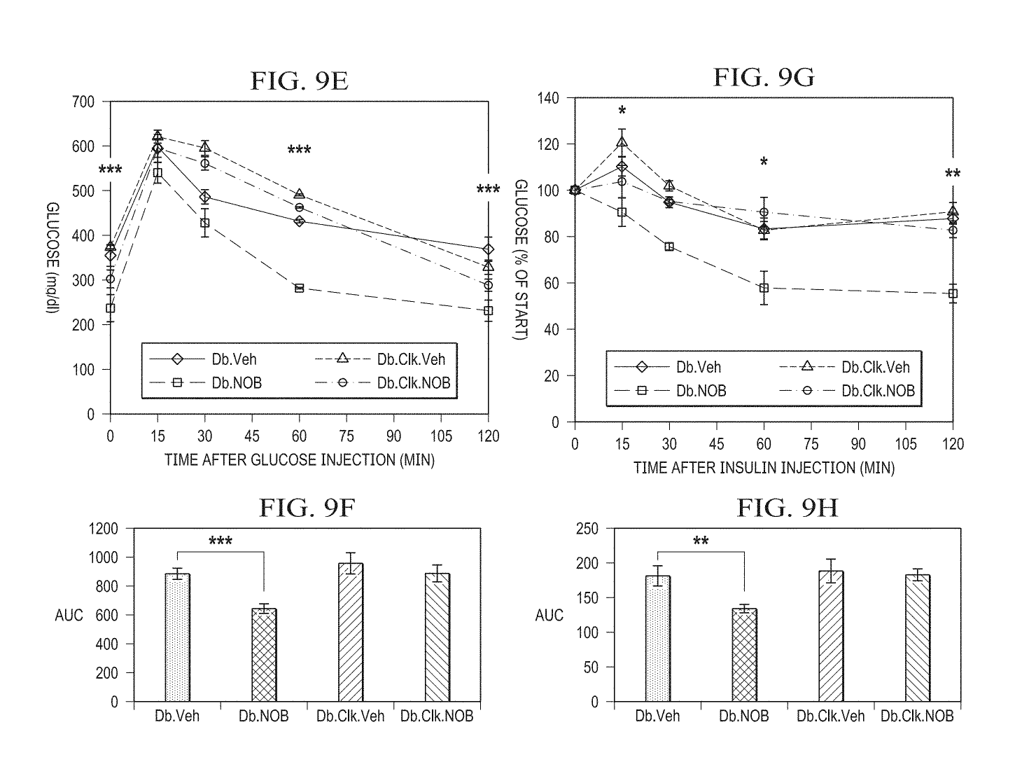

Given the improved glucose homeostasis in DIO mice treated with nobiletin, we next investigated effects of nobiletin on db/db mice, an established genetic mouse model for obesity and diabetes that lacks functional leptin receptors, and the role of the clock. nobiletin treatment strongly blunted body weight gain in db/db mice (FIG. 9A-9B), lowered fasting glucose levels (FIG. 9C-9D), improved glucose tolerance and insulin sensitivity (FIG. 9E-9H), and reduced serum total triglyceride (TG) and total cholesterol (TC) evels in db/db mice (FIGS. 9I and 9J). In contrast, db/db Clock^{Δ19/Δ19} double-mutant mice exhibited a markedly attenuated response to nobiletin (FIG. 9A-9K). Under vehicle treatment, db/db Clock^{Δ19/Δ19} mutant mice showed lower insulin levels than db/db, consistent with beta-cell deficits and hypoinsulinemia previously reported in Clock^{Δ19/Δ19} mice (Marcheva et al., 2010). Nobiletin treatment strongly reduced circulating insulin levels in both db/db and db/db Clock^{Δ19/Δ19} mutant mice, with the former exhibiting a more pronounced reduction (FIG. 9K). These results are consistent with lower insulin sensitivity in db/db Clock^{Δ19/Δ19} relative to db/db. Given the age-dependent deficits in pancreatic beta cell proliferation and insulin secretion in Clock^{Δ19/Δ19} (Marcheva et al., 2010), future studies will investigate the mechanistic relationship between the effects of nobiletin on insulin levels and beta cell function and/or insulin sensitivity.

### Example 4. Identification of Hepatic Nobiletin-Responsive Genes by Microarray

To characterize the molecular basis of nobiletin action in HFD-fed WT mice, studies focused on a major metabolic organ, the liver, in which we observed a strong protection by nobiletin was observed. Specifically, total RNAs prepared from liver tissues from RC.Veh and HF.Veh or HF.nobiletin WT mice for 10 weeks were reverse transcribed into cDNAs, which were then biotin-UTP labeled and hybridized to the Illumina mouse WG-6v2.0 Expression BeadChip. The data discussed in this publication have been deposited in the National Center for Biotechnology Information's Gene Expression Omnibus (GEO) and are accessible through GEO Series accession number GSE78848. Real-time qPCR and western blotting analyses of circadian gene expression were conducted as previously described (Yoo et al., 2013). Primers used were as follows:

| Gene Name | Forward Primer | Reverse Primer |
|---|---|---|
| mAdipq | TGTTCCTCTTAATCCTGCC | CCAACCTGCACAAGTTCCCTT |
| | CA (SEQ ID NO: 4) | (SEQ ID NO: 5) |
| mApoa1 | GGCACGTATGGCAGCAAG | CCAAGGAGGAGGATTCAAAC |
| | AT (SEQ ID NO: 6) | TG (SEQ ID NO: 7) |
| mApoa4 | CCAATGTGGTGTGGGATTA | AGTGACATCCGTCTTCTGAAA |
| | CTT (SEQ ID NO: 8) | C (SEQ ID NO: 9) |
| mApoc2 | ACCTGTACCAGAAGACAT | CCTGCGTAAGTGCTCATGG |
| | ACC (SEQ ID NO: 10) | (SEQ ID NO: 11) |
| mBmal1 | CCAAGAAAGTATGGACAC | GCATTCTTGATCCTTCCTTGGT |
| | AGACAAA (SEQ ID NO: 12) | (SEQ ID NO: 13) |
| mCD36 | CAAGCTCCTTGGCATGGTA | TGGATTTGCAAGCACAATATG |
| | GA (SEQ ID NO: 14) | AA (SEQ ID NO: 15) |
| mCidec | ATGGACTACGCCATGAAG | CGGTGCTAACACGACAGGG |
| | TCT (SEQ ID NO: 16) | (SEQ ID NO: 17) |
| mClock | CCTTCAGCAGTCAGTCCAT | AGACATCGCTGGCTGTGTTAA |
| | AAAC (SEQ ID NO: 18) | (SEQ ID NO: 19) |
| mCry1 | CTGGCGTGGAAGTCATCGT | CTGTCCGCCATTGAGTTCTAT |
| | (SEQ ID NO: 20) | G (SEQ ID NO: 21) |
| mCry2 | TGTCCCTTCCTGTGTGGAA | GCTCCCAGCTTGGCTTGA |
| | GA (SEQ ID NO: 22) | (SEQ ID NO: 23) |
| mCyp7a1 | GGGATTGCTGTGGTAGTG | GGTATGGAATCAACCCGTTGT |
| | AGC (SEQ ID NO: 24) | C (SEQ ID NO: 25) |
| mCyp7b1 | GGAGCCACGACCCTAGAT | TGCCAAGATAAGGAAGCCAA |
| | G (SEQ ID NO: 26) | C (SEQ ID NO: 27) |
| mDbp | CGTGGAGGTGCTTAATGA | CATGGCCTGGAATGCTTGA |
| | CCTTT (SEQ ID NO: 28) | (SEQ ID NO: 29) |
| mGAPDH | CAAGGTCATCCATGACAA | GGCCATCCACAGTCTTCTGG |
| | CTTTG (SEQ ID NO: 30) | (SEQ ID NO: 31) |
| mGCK | TGAGCCGGATGCAGAAGG | GCAACATCTTTACACTGGCCT |
| | A (SEQ ID NO: 32) | (SEQ ID NO: 33) |
| mGlut2 | TCAGAAGACAAGATCACC | GCTGGTGTGACTGTAAGTGGG |
| | GGA (SEQ ID NO: 34) | (SEQ ID NO: 35) |
| mIgfbp2 | CAGACGCTACGCTGCTATC | CCCTCAGAGTGGTCGTCATCA |
| | C (SEQ ID NO: 36) | (SEQ ID NO: 37) |
| mIkBa | GCACTTGGCAATCATCCAC | GTATTTCCTCGAAAGTCTCGG |
| | G (SEQ ID NO: 38) | AG (SEQ ID NO: 39) |
| mPdk4 | CCGCTGTCCATGAAGCA | GCAGAAAAGCAAAGGACGTT |
| | (SEQ ID NO: 40) | (SEQ ID NO: 41) |
| mPer1 | CCCAGCTTTACCTGCAGAA | ATGGTCGAAAGGAAGCCTCT |
| | G (SEQ ID NO: 42) | (SEQ ID NO: 43) |
| mPer2 | TGTGCGATGATGATTCGTG | GGTGAAGGTACGTTTGGTTTG |
| | A (SEQ ID NO: 44) | C (SEQ ID NO: 45) |
| mPGC-1α | TATGGAGTGACATAGAGT | CCACTTCAATCCACCCAGAAA |
| | GTGCT (SEQ ID NO: 46) | G (SEQ ID NO: 47) |
| mPkm2 | TGGATGTTGGCAAGGCCC | AGGGCCATCAAGGTACAGGC |
| | GA (SEQ ID NO: 48) | ACT (SEQ ID NO: 49) |
| mPlin2 | GACCTTGTGTCCTCCGCTT | CAACCGCAATTTGTGGCTC |
| | AT (SEQ ID NO: 50) | (SEQ ID NO: 51) |
| mPPARγ | CAAGAATACCAAAGTGCG | GAGCTGGGTCTTTTCAGAATA |
| | ATCAA (SEQ ID NO: 52) | ATAAG (SEQ ID NO: 53) |
| mRev-erbα | CATGGTGCTACTGTGTAAG | CACAGGCGTGCACTCCATAG |
| | GTGTGT (SEQ ID NO: 54) | (SEQ ID NO: 55) |
| mRev-erbβ | TGAACGCAGGAGGTGTGA | GAGGACTGGAAGCTATTCTCA |
| | TTG (SEQ ID NO: 56) | GA (SEQ ID NO: 57) |
| mRORα | GCACCTGACCGAAGACGA | GAGCGATCCGCTGACATCA |
| | AA (SEQ ID NO: 58) | (SEQ ID NO: 59) |
| mRORγ | TCAGCGCCCTGTGTTTTT | CGAGAACCAGGGCCGTGTAG |
| | (SEQ ID NO: 60) | (SEQ ID NO: 61) |
| mScd1 | TTCTTGCGATACACTCTGG | CGGGATTGAATGTTCTTGTCG |
| | TGC (SEQ ID NO: 62) | T (SEQ ID NO: 63) |
| mVldlr | GGCAGCAGGCAATGCAAT | GGGCTCGTCACTCCAGTCT |
| | G (SEQ ID NO: 64) | (SEQ ID NO: 65) |

For mammalian one-hybrid assays, regardless of the nature of the ligand (agonist or inverse agonist), ligand interaction with these chimeric receptors has been shown to reduce transcriptional activity of these chimeric receptors (Wang et al., 2010a, 2010b). For radio-ligand receptor binding assays, previously described protocols with minor modifications (Kumar et al., 2010; Wang et al., 2010b) were adopted.

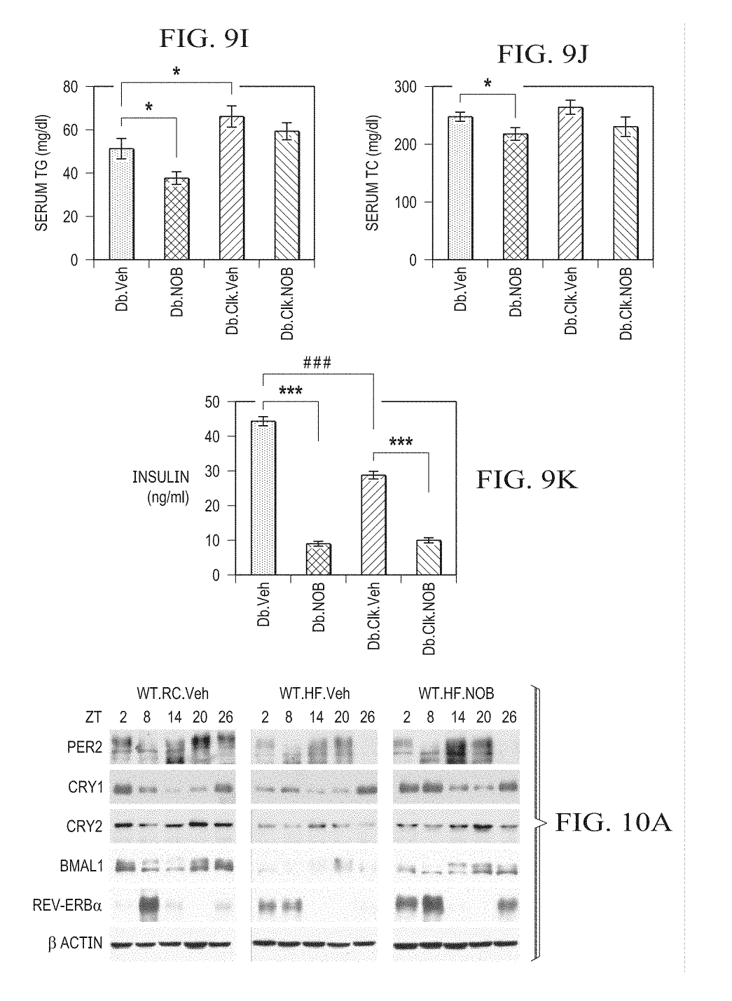

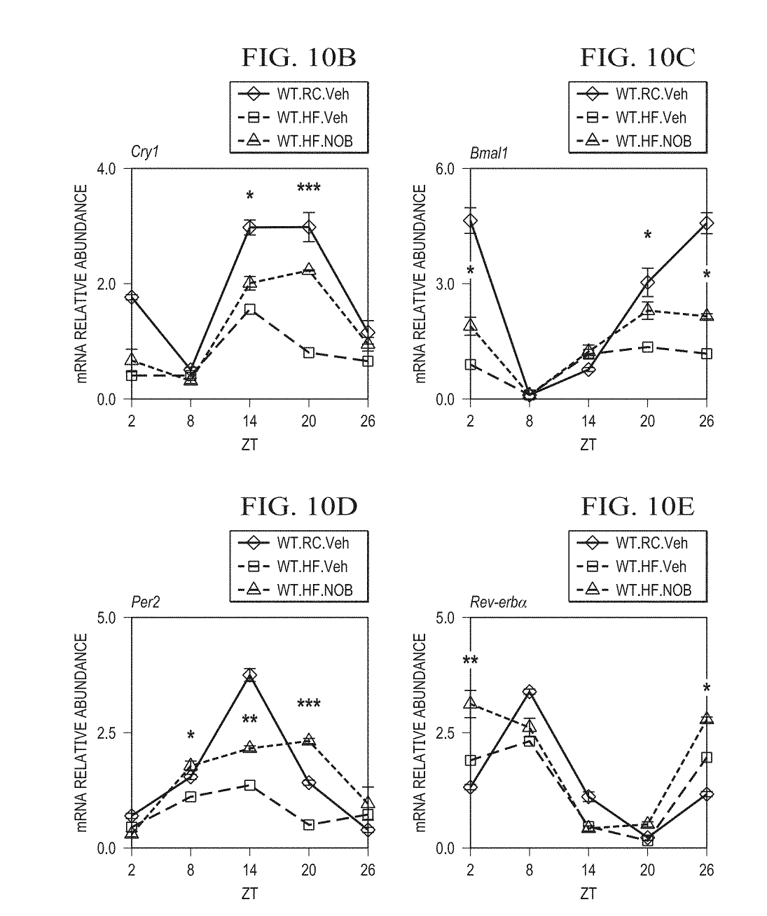

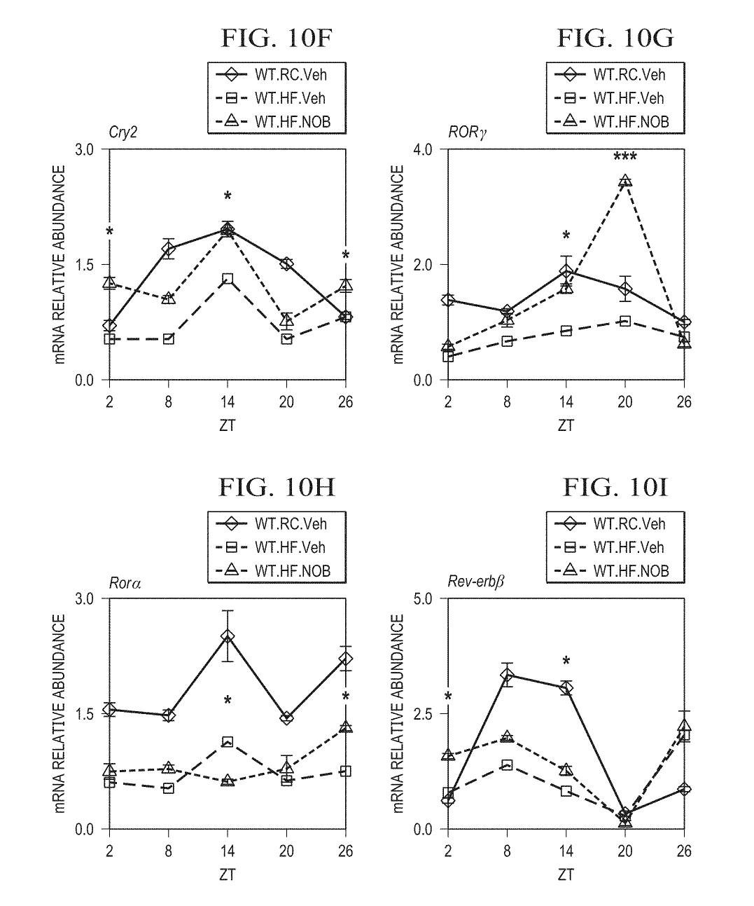

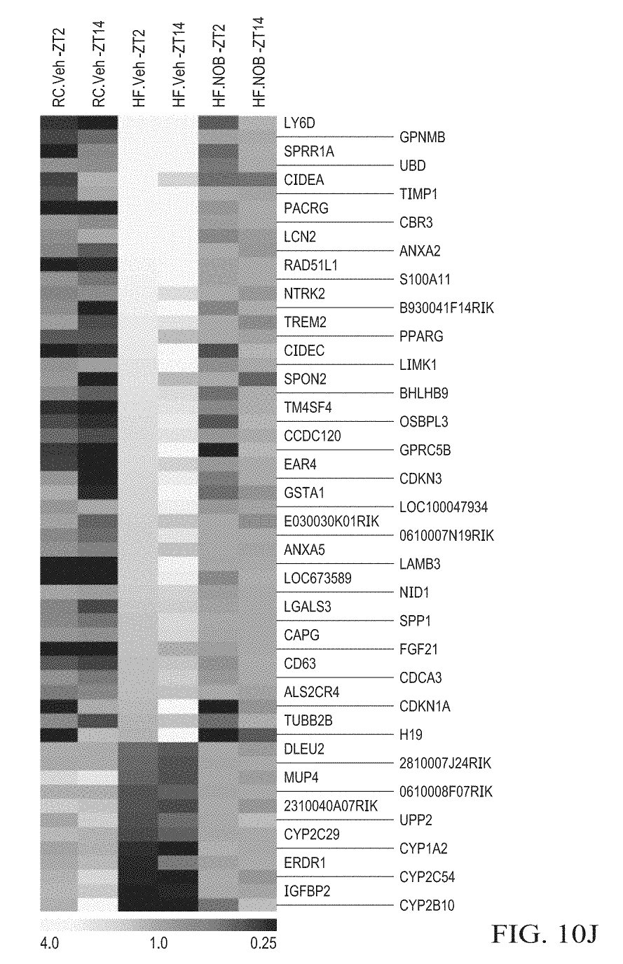

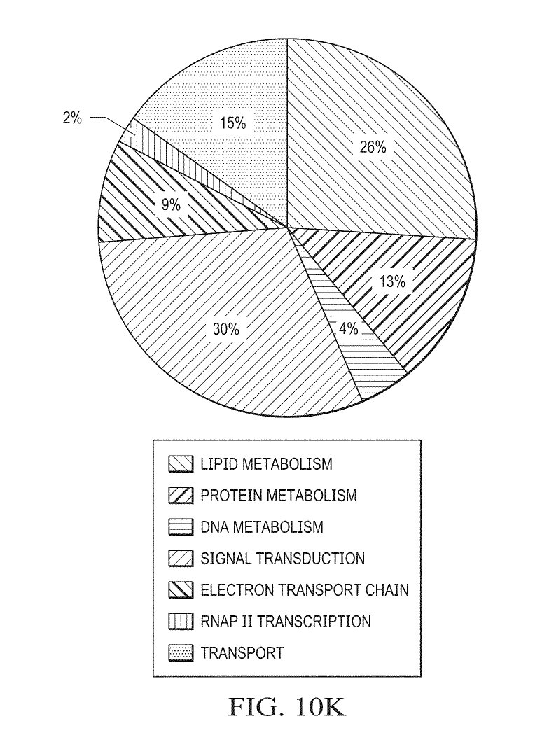

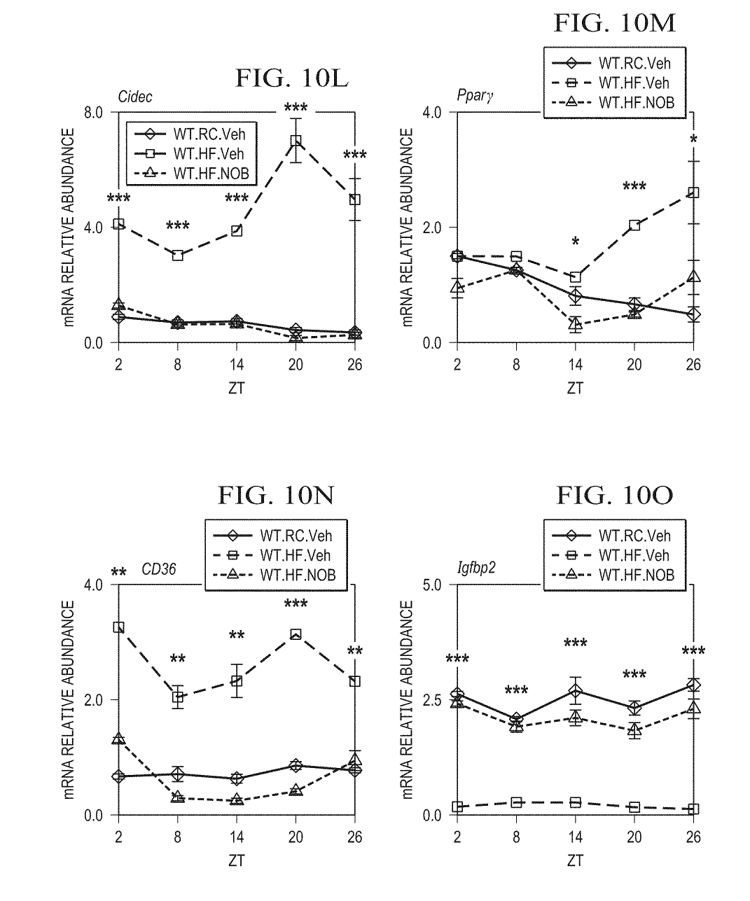

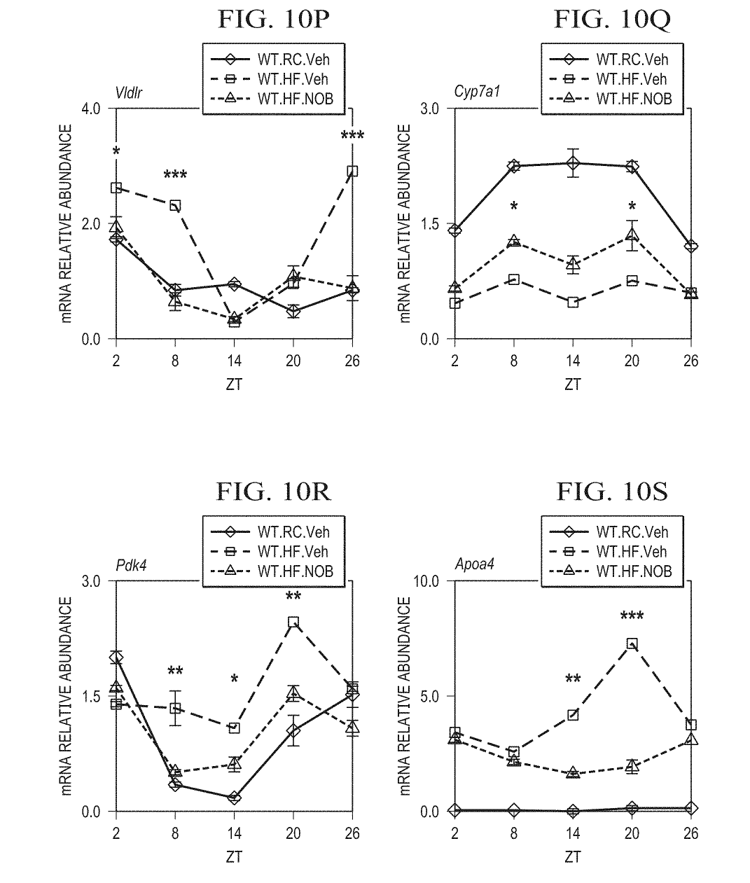

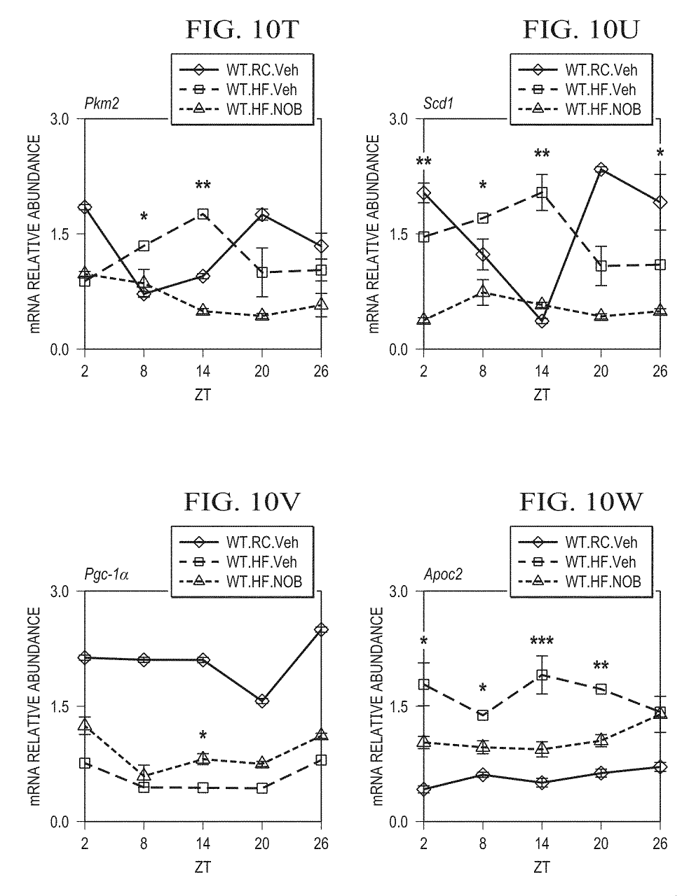

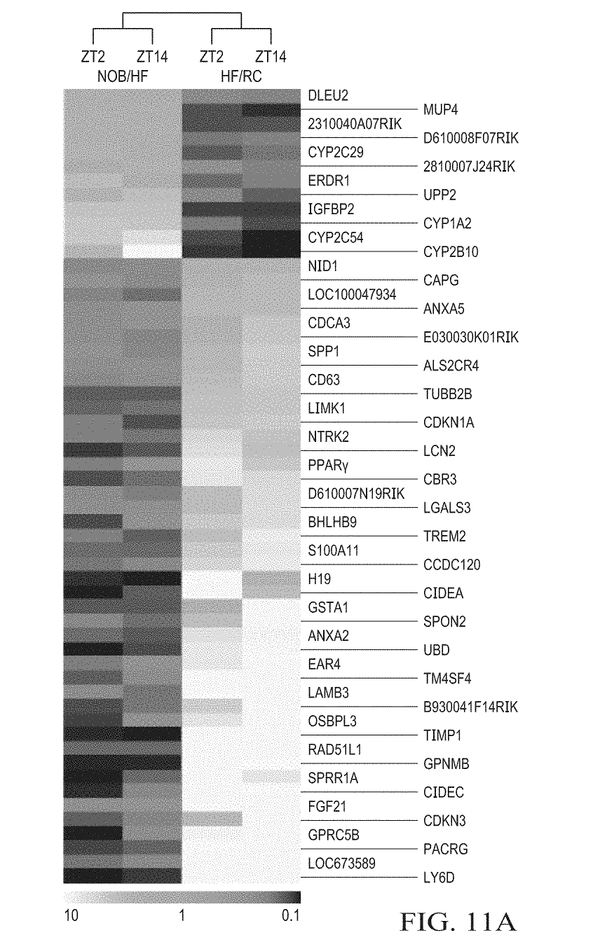

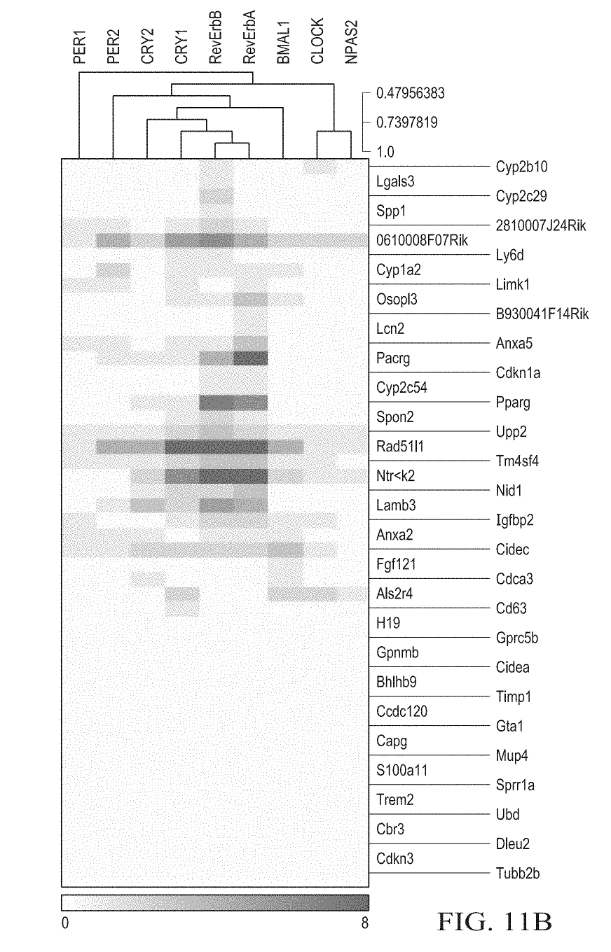

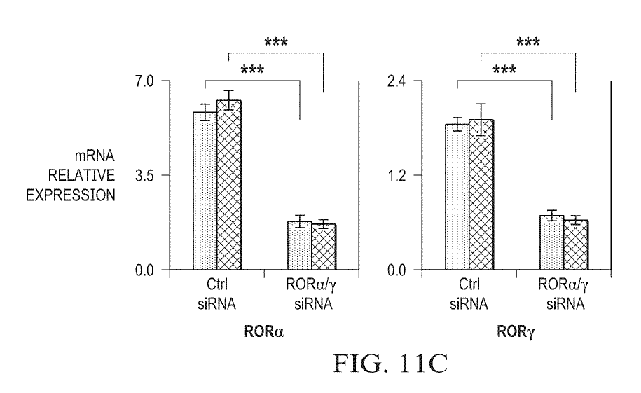

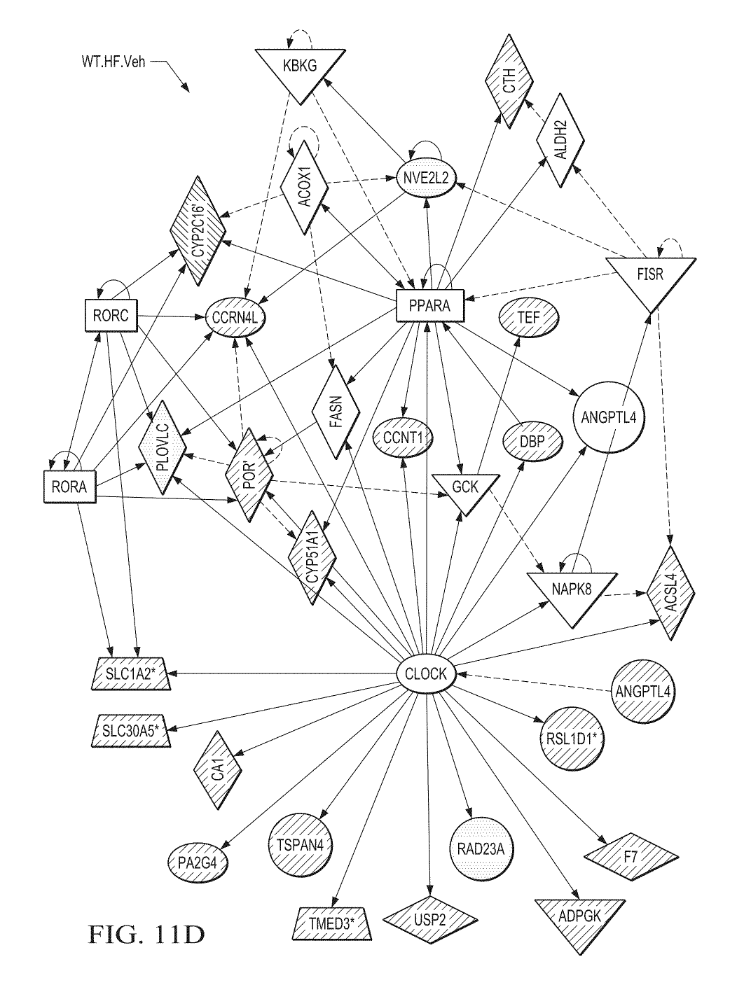

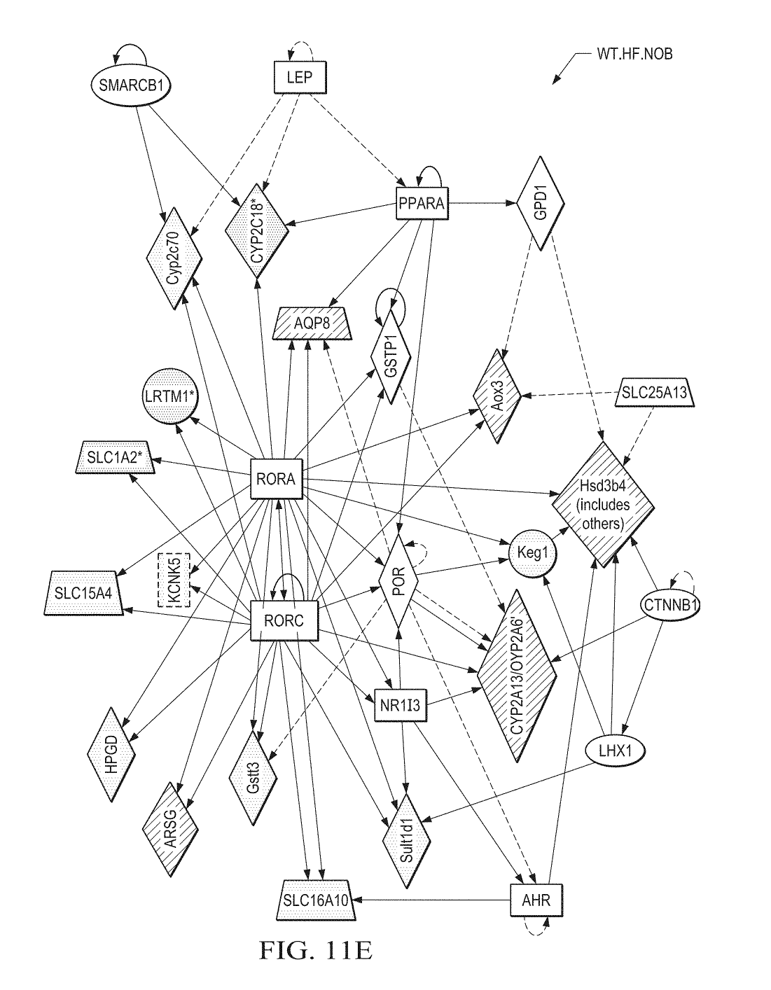

Consistent with previous studies (Kohsaka et al., 2007), the oscillatory amplitude of clock gene expression was generally lower in the liver of HFD-fed, vehicle-treated (HF.Veh) mice relative to lean RC-fed, vehicle-treated (RC.Veh) mice (FIGS. 10A-10I). Nobiletin improved circadian clock transcript oscillations and largely restored clock protein rhythms (FIGS. 10A-10I) (HFD-fed, nobiletin-treated [HF.nobiletin] versus HF.Veh mice), concordant with its robust physiological efficacy. To characterize metabolic output gene expression, microarray analysis was conducted. Comparative analysis was conducted to analyze gene expression changes in pairwise comparison, namely, HF.Veh versus RC.Veh (the comparison is denoted as HF/RC) and HF.nobiletin versus HF.Veh (denoted as nobiletin/HF), at time points ZT2 and ZT14, which revealed altered expression of 544, 229, 915, and 243 genes, respectively (data not shown). Importantly, a total of 251 and 56 genes were identified showing altered gene expression patterns in response to HFD (HF/RC) that were reversed by nobiletin (nobiletin/HF) at one or both time points (ZT2 and/or ZT14), respectively (FIGS. 10J and 11A; additional data not shown). Functional classification (Gene Ontology [GO]) of these nobiletin-responsive genes highlighted a prominent role in metabolic regulation (FIG. 10K; additional data not shown). Real-time quantitative PCR (qPCR) analysis further illustrated a broad modulatory function of nobiletin in metabolic output gene expression (FIG. 10L-10W). For example, transcript expression of Cidec/Fsp27, known to function in lipid droplet formation (Eckel-Mahan et al., 2013; Matsusue et al., 2008; Puri et al., 2007), was induced by >10-fold in HF.Veh mouse liver relative to RC.Veh and reverted back to baseline levels by nobiletin, consistent with the nobiletin efficacy in mitigating hepatic steatosis (FIG. 5L). Nobiletin also altered the expression of genes involved in gluconeogenesis and glycolysis (e.g., Pdk4 and Pkm2).

### Example 5. Retinoid Acid Receptor-Like Orphan Receptor (ROR) as Direct Protein Targets for Nobiletin

Cross-examination of previous circadian chromatin immunoprecipitation sequencing studies (Cho et al., 2012; Koike et al., 2012) revealed that 63% of the nobiletin-responsive genes showed promoter occupancy of core clock proteins (FIG. 11B), particularly REV-ERBs (protein encoded by the reverse DNA strand of c-erbA). REV-ERBs and RORs function respectively as negative and positive transcription factors competing for binding to RORE promoter elements, playing important roles in circadian rhythms, metabolism, and inflammation (Gerhart-Hines et al., 2013; Jetten et al., 2013; Kojetin and Burris, 2014). In a previous screen for inhibitors of RORγt, nobiletin was among a number of primary screen hits that instead activated RORγt and consequently were not validated further (Huh et al., 2011). ROR family receptors consist of α, β, and γ isoforms. RORα and RORγ are more similar in tissue distribution and the ligand-binding domain structure, whereas RORβ is more divergent (Kallen et al., 2002; Solt et al., 2010; Stehlin et al., 2001).

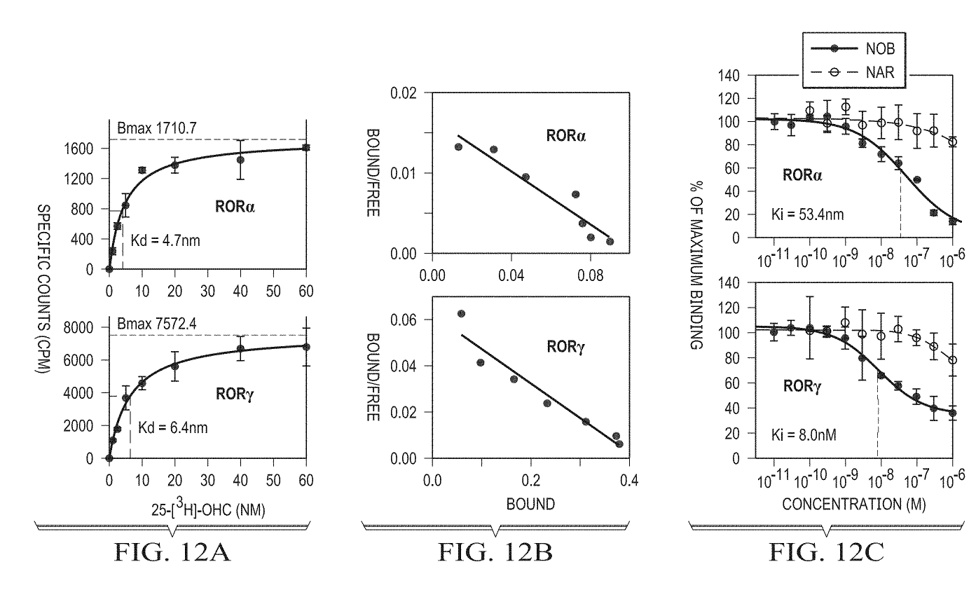

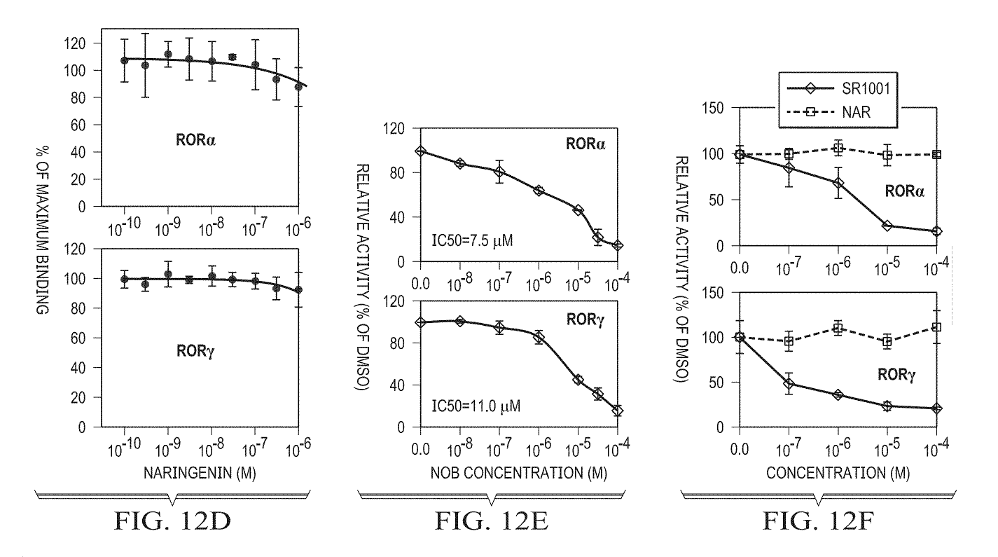

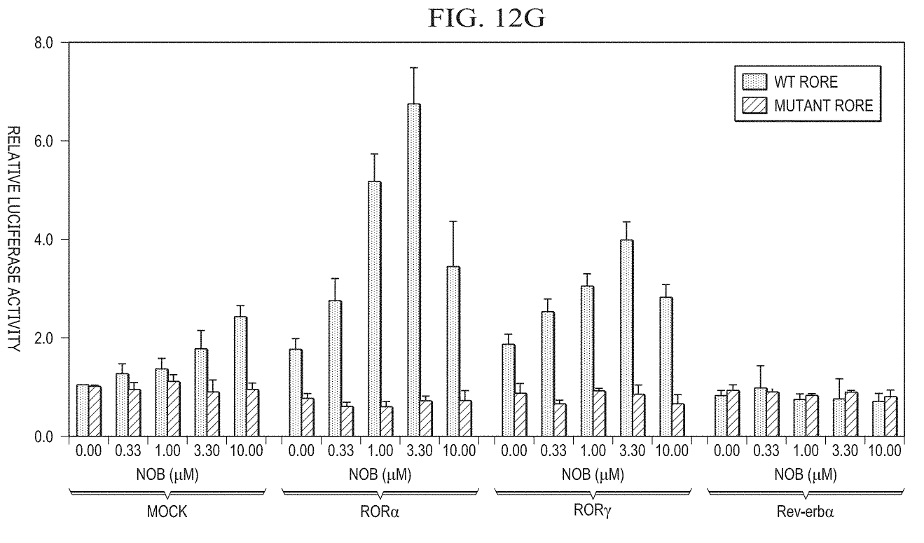

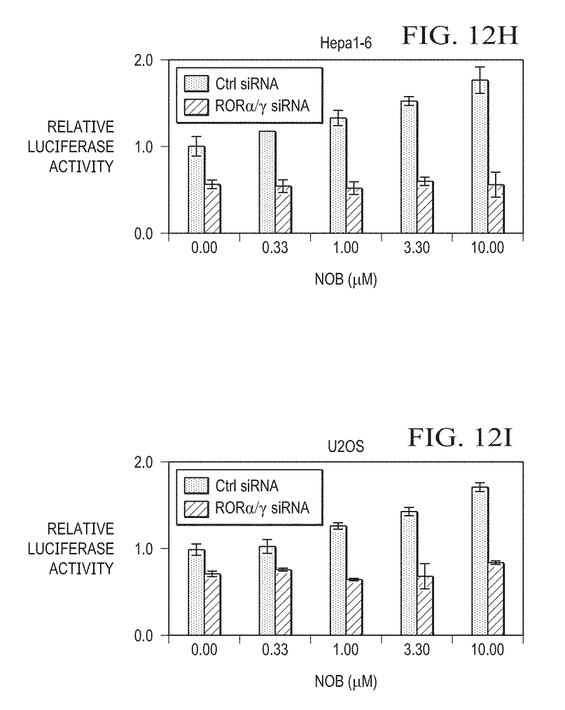

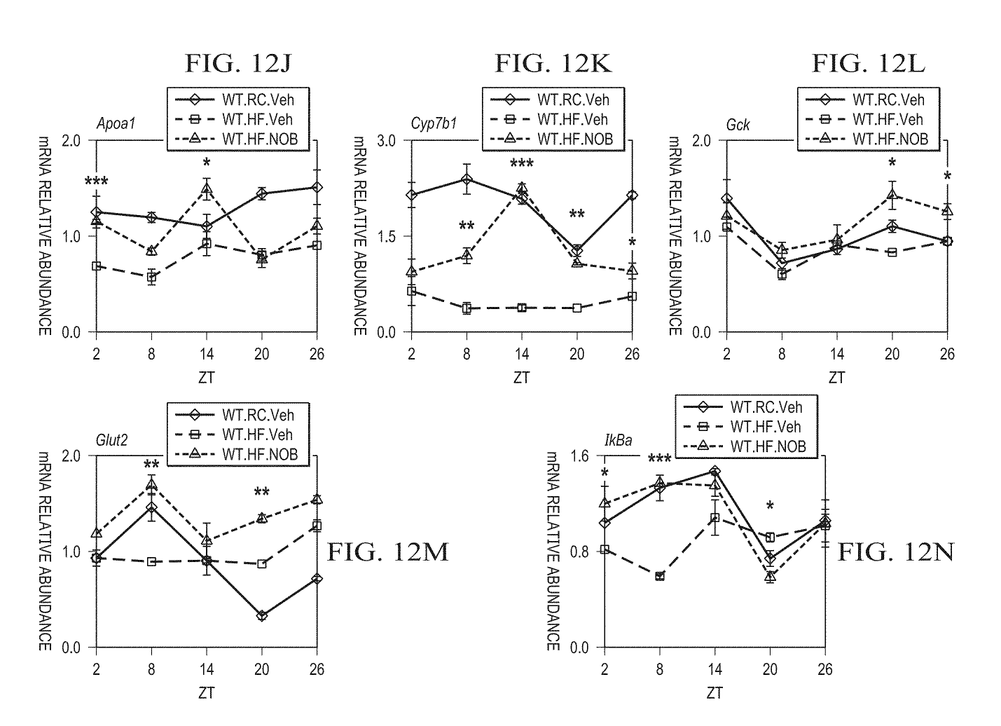

To characterize direct interaction between nobiletin and ROR proteins, a competitive radio-ligand binding assay for RORs using 25-[3H]-hydroxycholesterol (25-[3H]—OHC) (Kumar et al., 2010; Wang et al., 2010b). Saturation curves and Scatchard plots validated the assay, with similar Kd values to that previously reported (FIGS. 12A and 12B) (Kumar et al., 2010; Wang et al., 2010b) was used. Importantly, nobiletin showed robust competitive binding to the LBDs of RORα and RORγ, with higher affinity for RORγ (FIG. 12C; see Ki comparison). In contrast, naringin or its aglycone derivative Naringenin showed markedly diminished binding in the same concentration range (FIGS. 12C and 12D). Consistent with these binding assay results, nobiletin showed robust activities as did the known ROR ligand SR1001 (Solt et al., 2011) for GAL4-RORα and GAL4-RORγ chimeric receptors in mammalian one-hybrid reporter assays, and naringin showed no activities (FIGS. 12E and 12F). Of note, regardless of the nature of the ligand (agonist or inverse agonist), ligand interaction with these chimeric receptors has been shown to reduce transcriptional activity of these chimeric receptors (Wang et al., 2010a, 2010b). These results together indicate direct binding of nobiletin to RORα and RORγ.

Functional assays were used to characterize the effect of nobiletin on RORα/γ transcriptional activity. Nobiletin was found to dose-dependently increase Bmal1 promoter-driven luciferase reporter activity with WT, but not mutant, RORE elements (Preitner et al., 2002) in the presence of RORα or RORγ in Hepa1-6 cells (FIG. 12G). Conversely, knockdown of the Rora/c genes encoding RORα/γ by small interfering RNAs (siRNAs) abrogated the nobiletin-mediated induction of Bmal1 promoter-driven luciferase reporter activity in both Hepa1-6 and U2OS cells (FIGS. 12H-12N and 11C). Several ROR target genes (e.g., Cyp7b1, IkBa, and Gck) were induced in nobiletin-treated DIO mouse liver relative to control treatment (FIG. 12I), and Ingenuity pathway analysis also showed an important role of RORs in the genome-wide nobiletin response (FIG. 11D-11E). Together, these results indicate that nobiletin directly binds to and activates RORs and that RORα/γ are necessary for the enhancing activity of nobiletin on Bmal1 transcription.

### Summary of Examples

In summary, our unbiased chemical screen identified clock-enhancing polymethoxylated flavones, particularly nobiletin. Compelling evidence from both genetic and pharmacological studies demonstrates a Clock gene-dependent efficacy of nobiletin in preventing metabolic syndrome in mice, providing proof in mammals that strengthening circadian amplitude is a pharmacological intervention strategy for metabolic disease and other clock-related pathologies such as age-related decline. The beneficial outcome of enhanced circadian amplitude could include enhanced efficiency in physiological performance, greater stimuli range, and sensitized response indicating that augmented circadian amplitude enhances energy metabolism, time-restricted feeding, and thus energy expenditure, plays a dominant role determining extent of obesity from HFD feeding.

Polymethoxylated flavones elicit diverse benefits in mice and humans, including mitigating effects against cancer, inflammation, atherosclerosis, and more recently metabolic disorders and neurodegenerative diseases (Cui et al., 2010; Evans et al., 2012; Kurowska and Manthey, 2004; Lee et al., 2013; Mulvihill et al., 2009; Nohara et al., 2015a). Polymethoxylated flavones generally show a favorable pharmacokinetic profile (Evans et al., 2012; Saigusa et al., 2011), and no discernible toxicity was observed in chronic treatment of mice in this and previous studies (Lee et al., 2013; Mulvihill et al., 2011). The inventors' group showed a role of nobiletin in ammonia disposal via urea cycle regulation, and transcriptional induction of the rate-limiting Cps1 gene by nobiletin was impaired in Clock^{Δ19/Δ19} mutant mice (Nohara et al., 2015a). The present study illustrates a direct role of nobiletin in the enhancement of circadian clocks and particularly the activation of ROR receptors. These findings collectively indicate a unifying circadian mechanism governing the diverse physiological effects of polymethoxylated flavones. The ROR nuclear receptors were identified as the molecular target of nobiletin.

In conclusion, nobiletin is a clock-enhancing natural compound that activates RORs and protects against metabolic syndrome in a clock-dependent manner suggesting that such clock-enhancing compounds have application in other diseases (e.g., mood and sleep disorders) and aging.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Antoch, M. P., and Kondratov, R. V. (2013). Pharmacological modulators of the circadian clock as potential therapeutic drugs: focus on genotoxic/anticancer therapy. In Handbook of Experimental Pharmacology, W. Rosenthal, ed. (Springer), pp. 289-309.
Antoch, M. P., Song, E. J., Chang, A. M., Vitaterna, M. H., Zhao, Y., Wilsbacher, L. D., Sangoram, A. M., King, D. P., Pinto, L. H., and Takahashi, J. S. (1997). Functional identification of the mouse circadian Clock gene by transgenic BAC rescue. Cell 89, 655-667.
Asher, G., and Schibler, U. (2011). Crosstalk between components of circadian and metabolic cycles in mammals. Cell Metab. 13, 125-137.
Assini, J. M., Mulvihill, E. E., and Huff, M. W. (2013). Citrus flavonoids and lipid metabolism. Curr. Opin. Lipidol. 24, 34-40.
Bass, J., and Takahashi, J. S. (2010). Circadian integration of metabolism and energetics. Science 330, 1349-1354.
Ben-Aziz, A. (1967). Nobiletin is main fungistat in tangerines resistant to mal secco. Science 155, 1026-1027.
Buhr, E. D., Yoo, S. H., and Takahashi, J. S. (2010). Temperature as a universal resetting cue for mammalian circadian oscillators. Science 330, 379-385.
Chang, M. R., He, Y., Khan, T. M., Kuruvilla, D. S., Garcia-Ordonez, R., Corzo, C. A., Unger, T. J., White, D. W., Khan, S., Lin, L., et al. (2015). Antiobesity effect of a small molecule repressor of RORγ. Mol. Pharmacol. 88, 48-56.
Chen, Z., Yoo, S. H., and Takahashi, J. S. (2013). Small molecule modifiers of circadian clocks. Cell. Mol. Life Sci. 70, 2985-2998.
Chen, Z., Yoo, S. H., Park, Y. S., Kim, K. H., Wei, S., Buhr, E., Ye, Z. Y., Pan, H. L., and Takahashi, J. S. (2012). Identification of diverse modulators of central and peripheral circadian clocks by high-throughput chemical screening. Proc. Natl. Acad. Sci. USA 109, 101-106.
Cho, H., Zhao, X., Hatori, M., Yu, R. T., Barish, G. D., Lam, M. T., Chong, L. W., DiTacchio, L., Atkins, A. R., Glass, C. K., et al. (2012). Regulation of circadian behaviour and metabolism by REV-ERB-α and REV-ERB-β. Nature 485, 123-127.
Chutkow, W. A., Birkenfeld, A. L., Brown, J. D., Lee, H. Y., Frederick, D. W., Yoshioka, J., Patwari, P., Kursawe, R., Cushman, S. W., Plutzky, J., et al. (2010). Deletion of the alpha-arrestin protein Txnip in mice promotes adiposity and adipogenesis while preserving insulin sensitivity. Diabetes 59, 1424-1434.
Cui, Y., Wu, J., Jung, S. C., Park, D. B., Maeng, Y. H., Hong, J. Y., Kim, S. J., Lee, S. R., Kim, S. J., Kim, S. J., and Eun, S. Y. (2010). Anti-neuroinflammatory activity of nobiletin on suppression of microglial activation. Biol. Pharm. Bull. 33, 1814-1821.
Daniels, I. S., Zhang, J., O'Brien, W. G., 3rd, Tao, Z., Miki, T., Zhao, Z., Blackburn, M. R., and Lee, C. C. (2010). A role of erythrocytes in adenosine monophosphate initiation of hypometabolism in mammals. J. Biol. Chem. 285, 20716-20723.
Delerive, P., Monte', D., Dubois, G., Trottein, F., Fruchart-Najib, J., Mariani, J., Fruchart, J. C., and Staels, B. (2001). The orphan nuclear receptor ROR alpha is a negative regulator of the inflammatory response. EMBO Rep. 2, 42-48.
Dibner, C., Schibler, U., and Albrecht, U. (2010). The mammalian circadian timing system: organization and coordination of central and peripheral clocks. Annu. Rev. Physiol. 72, 517-549.
Eckel-Mahan, K. L., Patel, V. R., Mohney, R. P., Vignola, K. S., Baldi, P., and Sassone-Corsi, P. (2012). Coordination of the transcriptome and metabolome by the circadian clock. Proc. Natl. Acad. Sci. USA 109, 5541-5546.
Eckel-Mahan, K. L., Patel, V. R., de Mateo, S., Orozco-Solis, R., Ceglia, N. J., Sahar, S., Dilag-Penilla, S. A., Dyar, K. A., Baldi, P., and Sassone-Corsi, P. (2013). Reprogramming of the circadian clock by nutritional challenge. Cell 155, 1464-1478.
Edgar, R., Domrachev, M., and Lash, A. E. (2002). Gene Expression Omnibus: NCBI gene expression and hybridization array data repository. Nucleic Acids Res 30, 207-210.
Evans, M., Sharma, P., and Guthrie, N. (2012). Bioavailability of Citrus Polymethoxylated Flavones and Their Biological Role in Metabolic Syndrome and Hyperlipidemia. In Readings in Advanced Pharmacokinetics—Theory, Methods and Applications, A. Noreddin, ed. (InTech). http://dx.doi.org/10.5772/34087. http://www.intechopen.com/books/readings-in-advancedpharmacokinetics-theory-methods-and-applications/the-bioavailability-ofcitrus-polymethoxylated-flavones.
Farrow, S. N., Solari, R., and Willson, T. M. (2012). The importance of chronobiology to drug discovery. Expert Opin. Drug Discov. 7, 535-541.
Garcia, J. M., Scherer, T., Chen, J. A., Guillory, B., Nassif, A., Papusha, V., Smiechowska, J., Asnicar, M., Buettner, C., and Smith, R. G. (2013). Inhibition of cisplatin-induced lipid catabolism and weight loss by ghrelin in male mice. Endocrinology 154, 3118-3129.
Garidou, L., Pomié, C., Klopp, P., Waget, A., Charpentier, J., Aloulou, M., Giry, A., Serino, M., Stenman, L., Lahtinen, S., et al. (2015). The gut microbiota regulates intestinal CD4 T cells expressing RORγt and controls metabolic disease. Cell Metab. 22, 100-112.
Gerhart-Hines, Z., and Lazar, M. A. (2015). Circadian metabolism in the light of evolution. Endocr. Rev. 36, 289-304.
Gerhart-Hines, Z., Feng, D., Emmett, M. J., Everett, L. J., Loro, E., Briggs, E. R., Bugge, A., Hou, C., Ferrara, C., Seale, P., et al. (2013). The nuclear receptor Rev-erbα controls circadian thermogenic plasticity. Nature 503, 410-413.
Green, C. B., Takahashi, J. S., and Bass, J. (2008). The meter of metabolism. Cell 134, 728-742.
Hatori, M., Vollmers, C., Zarrinpar, A., DiTacchio, L., Bushong, E. A., Gill, S., Leblanc, M., Chaix, A., Joens, M., Fitzpatrick, J. A., et al. (2012). Timerestricted feeding without reducing caloric intake prevents metabolic diseases in mice fed a high-fat diet. Cell Metab. 15, 848-860.
He, B., Nohara, K., Ajami, N. J., Michalek, R. D., Tian, X., Wong, M., Losee-Olson, S. H., Petrosino, J. F., Yoo, S. H., Shimomura, K., and Chen, Z. (2015). Transmissible microbial and metabolomic remodeling by soluble dietary fiber improves metabolic homeostasis. Sci. Rep. 5, 10604.
Hirota, T., Lee, J. W., St John, P. C., Sawa, M., Iwaisako, K., Noguchi, T., Pongsawakul, P. Y., Sonntag, T., Welsh, D. K., Brenner, D. A., et al. (2012). Identification of small molecule activators of cryptochrome. Science 337, 1094-1097.
Hogenesch, J. B., and Herzog, E. D. (2011). Intracellular and intercellular processes determine robustness of the circadian clock. FEBS Lett. 585, 1427-1434.
Huh, J. R., Leung, M. W., Huang, P., Ryan, D. A., Krout, M. R., Malapaka, R. R., Chow, J., Manel, N., Ciofani, M., Kim, S. V., et al. (2011). Digoxin and its derivatives suppress TH17 cell differentiation by antagonizing RORγt activity. Nature 472, 486-490.
Ikeda, Y., Kumagai, H., Skach, A., Sato, M., and Yanagisawa, M. (2013). Modulation of circadian glucocorticoid oscillation via adrenal opioid-CXCR7 signaling alters emotional behavior. Cell 155, 1323-1336.
Isojima, Y., Nakajima, M., Ukai, H., Fujishima, H., Yamada, R. G., Masumoto, K. H., Kiuchi, R., Ishida, M., Ukai-Tadenuma, M., Minami, Y., et al. (2009). CKIepsilon/delta-dependent phosphorylation is a temperature-insensitive, period-determining process in the mammalian circadian clock. Proc. Natl. Acad. Sci. USA 106, 15744-15749.
Jeong, K., He, B., Nohara, K., Park, N., Shin, Y., Kim, S., Shimomura, K., Koike, N., Yoo, S. H., and Chen, Z. (2015). Dual attenuation of proteasomal and autophagic BMAL1 degradation in Clock D19/+ mice contributes to improved glucose homeostasis. Sci. Rep. 5, 12801.
Jetten, A. M. (2009). Retinoid-related orphan receptors (RORs): critical roles in development, immunity, circadian rhythm, and cellular metabolism. Nucl. Recept. Signal. 7, e003.
Jetten, A. M., Kang, H. S., and Takeda, Y. (2013). Retinoic acid-related orphan receptors a and g: key regulators of lipid/glucose metabolism, inflammation, and insulin sensitivity. Front. Endocrinol. (Lausanne) 4, 1.
Kallen, J. A., Schlaeppi, J. M., Bitsch, F., Geisse, S., Geiser, M., Delhon, I., and Fournier, B. (2002). X-ray structure of the hRORalpha LBD at 1.63 A: structural and functional data that cholesterol or a cholesterol derivative is the natural ligand of RORalpha. Structure 10, 1697-1707.
King, D. P., Zhao, Y., Sangoram, A. M., Wilsbacher, L. D., Tanaka, M., Antoch, M. P., Steeves, T. D., Vitaterna, M. H., Kornhauser, J. M., Lowrey, P. L., et al. (1997). Positional cloning of the mouse circadian clock gene. Cell 89, 641-653.
Knight, Z. A., and Shokat, K. M. (2007). Chemical genetics: where genetics and pharmacology meet. Cell 128, 425-430.
Kohsaka, A., Laposky, A. D., Ramsey, K. M., Estrada, C., Joshu, C., Kobayashi, Y., Turek, F. W., and Bass, J. (2007). High-fat diet disrupts behavioral and molecular circadian rhythms in mice. Cell Metab. 6, 414-421.
Koike, N., Yoo, S. H., Huang, H. C., Kumar, V., Lee, C., Kim, T. K., and Takahashi, J. S. (2012). Transcriptional architecture and chromatin landscape of the core circadian clock in mammals. Science 338, 349-354.
Kojetin, D. J., and Burris, T. P. (2014). REV-ERB and ROR nuclear receptors as drug targets. Nat. Rev. Drug Discov. 13, 197-216.
Kumar, A., Devaraj, V. C., Giri, K. C., Giri, S., Rajagopal, S., and Mullangi, R. (2012). Development and validation of a highly sensitive LC-MS/MS-ESI method for the determination of nobiletin in rat plasma: application to a pharmacokinetic study. Biomed Chromatogr 26, 1464-1471.
Kumar, N., Solt, L. A., Conkright, J. J., Wang, Y., Istrate, M. A., Busby, S. A., Garcia-Ordonez, R. D., Burris, T. P., and Griffin, P. R. (2010). The benzenesulfonamide T0901317 [N-(2,2,2-trifluoroethyl)-N-[4-[2,2,2-trifluoro-1-hydroxy-1-(trifluoromethyl)ethyl]phenyl]-benzenesulfonamide] is a novel retinoic acid receptor-related orphan receptor-alpha/gamma inverse agonist. Mol. Pharmacol. 77, 228-236.
Kurowska, E. M., and Manthey, J. A. (2004). Hypolipidemic effects and absorption of citrus polymethoxylated flavones in hamsters with diet-induced hypercholesterolemia. J. Agric. Food Chem. 52, 2879-2886.
Lau, P., Fitzsimmons, R. L., Raichur, S., Wang, S. C., Lechtken, A., and Muscat, G. E. (2008). The orphan nuclear receptor, RORalpha, regulates gene expression that controls lipid metabolism: staggerer (SG/SG) mice are resistant to diet-induced obesity. J. Biol. Chem. 283, 18411-18421.
Lee, C., Etchegaray, J. P., Cagampang, F. R., Loudon, A. S., and Reppert, S. M. (2001). Posttranslational mechanisms regulate the mammalian circadian clock. Cell 107, 855-867. Lee, Y. S., Cha, B. Y., Choi, S. S., Choi, B. K., Yonezawa, T., Teruya, T., Nagai, K., and Woo, J. T. (2013). Nobiletin improves obesity and insulin resistance in high-fat diet-induced obese mice. J Nutr Biochem 24, 156-162.
Lee, Y., Chen, R., Lee, H. M., and Lee, C. (2011). Stoichiometric relationship among clock proteins determines robustness of circadian rhythms. J. Biol. Chem. 286, 7033-7042.
Lee, Y. S., Cha, B. Y., Choi, S. S., Choi, B. K., Yonezawa, T., Teruya, T., Nagai, K., and Woo, J. T. (2013). Nobiletin improves obesity and insulin resistance in high-fat diet-induced obese mice. J. Nutr. Biochem. 24, 156-162.
Lee, Y. S., Cha, B. Y., Saito, K., Yamakawa, H., Choi, S. S., Yamaguchi, K., Yonezawa, T., Teruya, T., Nagai, K., and Woo, J. T. (2010). Nobiletin improves hyperglycemia and insulin resistance in obese diabetic ob/ob mice. Biochem. Pharmacol. 79, 1674-1683.
Li, R. W., Theriault, A. G., Au, K., Douglas, T. D., Casaschi, A., Kurowska, E. M., and Mukherjee, R. (2006). Citrus polymethoxylated flavones improve lipid and glucose homeostasis and modulate adipocytokines in fructose-induced insulin resistant hamsters. Life Sci 79, 365-373.
Liu, A. C., Welsh, D. K., Ko, C. H., Tran, H. G., Zhang, E. E., Priest, A. A., Buhr, E. D., Singer, O., Meeker, K., Verma, I. M., et al. (2007). Intercellular coupling confers robustness against mutations in the SCN circadian clock network. Cell 129, 605-616.
Liu, Y., Zhou, D., Zhang, F., Tu, Y., Xia, Y., Wang, H., Zhou, B., Zhang, Y., Wu, J., Gao, X., et al. (2012). Liver Patt1 deficiency protects male mice from age-associated but not high-fat dietinduced hepatic steatosis. Journal of lipid research 53, 358-367.
Mamontova, A., Séguret-Macé, S., Esposito, B., Chaniale, C., Bouly, M., Delhaye-Bouchaud, N., Luc, G., Staels, B., Duverger, N., Mariani, J., and Tedgui, A. (1998). Severe atherosclerosis and hypoalphalipoproteinemia in the staggerer mouse, a mutant of the nuclear receptor RORalpha. Circulation 98, 2738-2743.
Marcheva, B., Ramsey, K. M., Buhr, E. D., Kobayashi, Y., Su, H., Ko, C. H., Ivanova, G., Omura, C., Mo, S., Vitaterna, M. H., et al. (2010). Disruption of the clock components CLOCK and BMAL1 leads to hypoinsulinaemia and diabetes. Nature 466, 627-631.
Matsusue, K., Kusakabe, T., Noguchi, T., Takiguchi, S., Suzuki, T., Yamano, S., and Gonzalez, F. J. (2008). Hepatic steatosis in leptin-deficient mice is promoted by the PPARgamma target gene Fsp27. Cell Metab. 7, 302-311.
Meng, Q. J., Maywood, E. S., Bechtold, D. A., Lu, W. Q., Li, J., Gibbs, J. E., Dupré, S. M., Chesham, J. E., Rajamohan, F., Knafels, J., et al. (2010). Entrainment of disrupted circadian behavior through inhibition of casein kinase 1 (CK1) enzymes. Proc. Natl. Acad. Sci. USA 107, 15240-15245.
Mulvihill, E. E., Allister, E. M., Sutherland, B. G., Telford, D. E., Sawyez, C. G., Edwards, J. Y., Markle, J. M., Hegele, R. A., and Huff, M. W. (2009). Naringenin prevents dyslipidemia, apolipoprotein B overproduction, and hyperinsulinemia in LDL receptor-null mice with diet-induced insulin resistance. Diabetes 58, 2198-2210.
Mulvihill, E. E., Assini, J. M., Lee, J. K., Allister, E. M., Sutherland, B. G., Koppes, J. B., Sawyez, C. G., Edwards, J. Y., Telford, D. E., Charbonneau, A., et al. (2011). Nobiletin attenuates VLDL overproduction, dyslipidemia, and atherosclerosis in mice with diet-induced insulin resistance. Diabetes 60, 1446-1457.
Nagase, H., Yamakuni, T., Matsuzaki, K., Maruyama, Y., Kasahara, J., Hinohara, Y., Kondo, S., Mimaki, Y., Sashida, Y., Tank, A. W., et al. (2005). Mechanism of neurotrophic action of nobiletin in PC12D cells. Biochemistry 44, 13683-13691.
Nohara, K., Shin, Y., Park, N., Jeong, K., He, B., Koike, N., Yoo, S. H., and Chen, Z. (2015a). Ammonia-lowering activities and carbamoyl phosphate synthetase 1 (Cps1) induction mechanism of a natural flavonoid. Nutr. Metab. (Lond.) 12, 23.
Nohara, K., Yoo, S. H., and Chen, Z. J. (2015b). Manipulating the circadian and sleep cycles to protect against metabolic disease. Front. Endocrinol. (Lausanne) 6, 35.
Ohnmacht, C., Park, J. H., Cording, S., Wing, J. B., Atarashi, K., Obata, Y., Gaboriau-Routhiau, V., Marques, R., Dulauroy, S., Fedoseeva, M., et al. (2015). MUCOSAL IMMUNOLOGY. The microbiota regulates type 2 immunity through RORγt+ T cells. Science 349, 989-993.
Okada, S., Markle, J. G., Deenick, E. K., Mele, F., Averbuch, D., Lagos, M., Alzahrani, M., Al-Muhsen, S., Halwani, R., Ma, C. S., et al. (2015). Immunodeficiencies. Impairment of immunity to Candida and Mycobacterium in humans with bi-allelic RORC mutations. Science 349, 606-613.
Preitner, N., Damiola, F., Lopez-Molina, L., Zakany, J., Duboule, D., Albrecht, U., and Schibler, U. (2002). The orphan nuclear receptor REV-ERBalpha controls circadian transcription within the positive limb of the mammalian circadian oscillator. Cell 110, 251-260.
Puri, V., Konda, S., Ranjit, S., Aouadi, M., Chawla, A., Chouinard, M., Chakladar, A., and Czech, M. P. (2007). Fat-specific protein 27, a novel lipid droplet protein that enhances triglyceride storage. J. Biol. Chem. 282, 34213-34218.
Raichur, S., Fitzsimmons, R. L., Myers, S. A., Pearen, M. A., Lau, P., Eriksson, N., Wang, S. M., and Muscat, G. E. (2010). Identification and validation of the pathways and functions regulated by the orphan nuclear receptor, ROR alpha1, in skeletal muscle. Nucleic Acids Res. 38, 4296-4312.
Roenneberg, T., Allebrandt, K. V., Merrow, M., and Vetter, C. (2012). Social jetlag and obesity. Curr. Biol. 22, 939-943.
Roza, J. M., Xian-Liu, Z., and Guthrie, N. (2007). Effect of citrus flavonoids and tocotrienols on serum cholesterol levels in hypercholesterolemic subjects. Altern. Ther. Health Med. 13, 44-48.
Rutter, J., Reick, M., and McKnight, S. L. (2002). Metabolism and the control of circadian rhythms. Annu. Rev. Biochem. 71, 307-331.
Saigusa, D., Shibuya, M., Jinno, D., Yamakoshi, H., Iwabuchi, Y., Yokosuka, A., Mimaki, Y., Naganuma, A., Ohizumi, Y., Tomioka, Y., and Yamakuni, T. (2011). High-performance liquid chromatography with photodiode array detection for determination of nobiletin content in the brain and serum of mice administrated the natural compound. Anal. Bioanal. Chem. 400, 3635-3641.
Santori, F. R., Huang, P., van de Pavert, S. A., Douglass, E. F., Jr., Leaver, D. J., Haubrich, B. A., Keber, R., Lorbek, G., Konijn, T., Rosales, B. N., et al. (2015). Identification of natural RORg ligands that regulate the development of lymphoid cells. Cell Metab. 21, 286-297.
Sato, T. K., Panda, S., Miraglia, L. J., Reyes, T. M., Rudic, R. D., McNamara, P., Naik, K. A., FitzGerald, G. A., Kay, S. A., and Hogenesch, J. B. (2004). A functional genomics strategy reveals Rora as a component of the mammalian circadian clock. Neuron 43, 527-537.
Scheer, F. A., Hilton, M. F., Mantzoros, C. S., and Shea, S. A. (2009). Adverse metabolic and cardiovascular consequences of circadian misalignment. Proc. Natl. Acad. Sci. USA 106, 4453-4458.
Schmid, B., Helfrich-Forster, C., and Yoshii, T. (2011). A new ImageJ plug-in “ActogramJ” for chronobiological analyses. J Biol Rhythms 26, 464-467.
Schroeder, A. M., and Colwell, C. S. (2013). How to fix a broken clock. Trends Pharmacol. Sci. 34, 605-619.
Shi, S. Q., Ansari, T. S., McGuinness, O. P., Wasserman, D. H., and Johnson, C. H. (2013). Circadian disruption leads to insulin resistance and obesity. Curr. Biol. 23, 372-381.
Singh, S. P., Wahajuddin, Tewari, D., Patel, K., and Jain, G. K. (2011). Permeability determination and pharmacokinetic study of nobiletin in rat plasma and brain by validated high performance liquid chromatography method. Fitoterapia 82, 1206-1214.
Solt, L. A., Griffin, P. R., and Burris, T. P. (2010). Ligand regulation of retinoic acid receptor-related orphan receptors: implications for development of novel therapeutics. Curr. Opin. Lipidol. 21, 204-211.
Solt, L. A., Kumar, N., Nuhant, P., Wang, Y., Lauer, J. L., Liu, J., Istrate, M. A., Kamenecka, T. M., Roush, W. R., Vidovic, D., et al. (2011). Suppression of TH17 differentiation and autoimmunity by a synthetic ROR ligand. Nature 472, 491-494.
Solt, L. A., Wang, Y., Banerjee, S., Hughes, T., Kojetin, D. J., Lundasen, T., Shin, Y., Liu, J., Cameron, M. D., Noel, R., et al. (2012). Regulation of circadian behaviour and metabolism by synthetic REV-ERB agonists. Nature 485, 62-68.
Stapleton, C. M., Jaradat, M., Dixon, D., Kang, H. S., Kim, S. C., Liao, G., Carey, M. A., Cristiano, J., Moorman, M. P., and Jetten, A. M. (2005). Enhanced susceptibility of staggerer (RORalphasg/sg) mice to lipopolysaccharide-induced lung inflammation. Am. J. Physiol. Lung Cell. Mol. Physiol. 289, L144-L152.
Stehlin, C., Wurtz, J. M., Steinmetz, A., Greiner, E., Schille, R., Moras, D., and Renaud, J. P. (2001). X-ray structure of the orphan nuclear receptor RORbeta ligand-binding domain in the active conformation. EMBO J. 20, 5822-5831.
Takahashi, J. S., Hong, H. K., Ko, C. H., and McDearmon, E. L. (2008). The genetics of mammalian circadian order and disorder: implications for physiology and disease. Nat. Rev. Genet. 9, 764-775.
Turek, F. W., Joshu, C., Kohsaka, A., Lin, E., Ivanova, G., McDearmon, E., Laposky, A., Losee-Olson, S., Easton, A., Jensen, D. R., et al. (2005). Obesity and metabolic syndrome in circadian Clock mutant mice. Science 308, 1043-1045.
van Ooijen, G., and Millar, A. J. (2012). Non-transcriptional oscillators in circadian timekeeping. Trends Biochem. Sci. 37, 484-492.
Vitaterna, M. H., D. P. King, A.-M. Chang, J. M. Kornhauser, P. L. Lowrey, J. D. McDonald, W. F. Dove, L. H. Pinto, F. W. Turek and J. S. Takahashi. (1994). Mutagenesis and mapping of a mouse gene, Clock, essential for circadian behavior. Science 264, 719-725.
Wallach, T., and Kramer, A. (2015). Chemical chronobiology: Toward drugs manipulating time. FEBS Lett. 589, 1530-1538.
Walle, T. (2007). Methoxylated flavones, a superior cancer chemopreventive flavonoid subclass? Semin. Cancer Biol. 17, 354-362.
Wang, Y., Kumar, N., Nuhant, P., Cameron, M. D., Istrate, M. A., Roush, W. R., Griffin, P. R., and Burris, T. P. (2010a). Identification of SR1078, a synthetic agonist for the orphan nuclear receptors RORalpha and RORgamma. ACS Chem. Biol. 5, 1029-1034.
Wang, Y., Kumar, N., Solt, L. A., Richardson, T. I., Helvering, L. M., Crumbley, C., Garcia-Ordonez, R. D., Stayrook, K. R., Zhang, X., Novick, S., et al. (2010b). Modulation of retinoic acid receptor-related orphan receptor alpha and gamma activity by 7-oxygenated sterol ligands. J. Biol. Chem. 285, 5013-5025.
Xiao, S., Yosef, N., Yang, J., Wang, Y., Zhou, L., Zhu, C., Wu, C., Baloglu, E., Schmidt, D., Ramesh, R., et al. (2014). Small-molecule RORγt antagonists inhibit T helper 17 cell transcriptional network by divergent mechanisms. Immunity 40, 477-489.
Yagita, K., Tamanini, F., Yasuda, M., Hoeijmakers, J. H., van der Horst, G. T., and Okamura, H. (2002). Nucleocytoplasmic shuttling and mCRY-dependent inhibition of ubiquitylation of the mPER2 clock protein. EMBO J. 21, 1301-1314.
Yang, X., Downes, M., Yu, R. T., Bookout, A. L., He, W., Straume, M., Mangelsdorf, D. J., and Evans, R. M. (2006). Nuclear receptor expression links the circadian clock to metabolism. Cell 126, 801-810.
Yoo, S. H., Mohawk, J. A., Siepka, S. M., Shan, Y., Huh, S. K., Hong, H. K., Kornblum, I., Kumar, V., Koike, N., Xu, M., et al. (2013). Competing E3 ubiquitin ligases govern circadian periodicity by degradation of CRY in nucleus and cytoplasm. Cell 152, 1091-1105.
Zhang, E. E., and Kay, S. A. (2010). Clocks not winding down: unravelling circadian networks. Nat. Rev. Mol. Cell Biol. 11, 764-776.
Zhang, R., Lahens, N. F., Ballance, H. I., Hughes, M. E., and Hogenesch, J. B. (2014). A circadian gene expression atlas in mammals: implications for biology and medicine. Proc. Natl. Acad. Sci. USA 111, 16219-16224.
Zheng, B., Larkin, D. W., Albrecht, U., Sun, Z. S., Sage, M., Eichele, G., Lee, C. C., and Bradley, A. (1999). The mPer2 gene encodes a functional component of the mammalian circadian clock. Nature 400, 169-173.

What is claimed is:

1. A method, comprising:
administering, to a mammal suffering from or at risk of suffering from metabolic syndrome or a constituent condition thereof, a composition comprising at least one polymethoxylated flavone.

2. The method of claim 1, wherein the at least one polymethoxylated flavone has formula I: 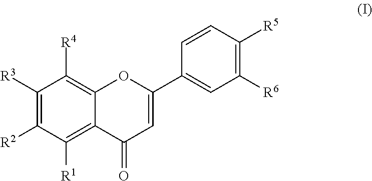
wherein R¹, R², R³, R⁴, R⁵, and R⁶ are each independently —H or —OCH₃, provided at least two of R¹, R², R³, R⁴, R⁵, and R⁶ are —OCH₃.

3. The method of claim 2, wherein the at least one polymethoxylated flavone is selected from the group consisting of nobiletin, tangeretin, and both.

4. The method of claim 1, wherein administering is at a dosage from 20 mg polymethoxylated flavone/kg body weight of the mammal to 2000 mg polymethoxylated flavone/kg body weight of the mammal and a rate from twice per day to once per week for a duration of at least one week.

5. The method of claim 4, wherein administering is at a dosage of 200 mg polymethoxylated flavone/kg body weight of the mammal, a rate of once every two days, and a duration of at least ten weeks.

6. The method of claim 1, wherein administering is by an oral route.

7. The method of claim 6, wherein the composition is in a form selected from the group consisting of liquids, tablets, capsules, caplets, and soluble powders.

8. A method, comprising:
administering, to a mammal suffering from or at risk of suffering from a sleep disorder, a composition comprising at least one polymethoxylated flavone.

9. The method of claim 8, wherein the at least one polymethoxylated flavone has formula I: 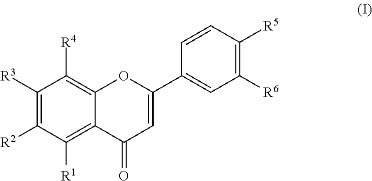
wherein R¹, R², R³, R⁴, R⁵, and R⁶ are each independently —H or —OCH₃, provided at least two of R¹, R², R³, R⁴, R⁵, and R⁶ are —OCH₃.

10. The method of claim 9, wherein the at least one polymethoxylated flavone is selected from the group consisting of nobiletin, tangeretin, and both.

11. The method of claim 8, wherein administering is at a dosage from 20 mg polymethoxylated flavone/kg body weight of the mammal to 2000 mg polymethoxylated flavone/kg body weight of the mammal and a rate from twice per day to once per week.

12. The method of claim 11, wherein administering is at a dosage of 200 mg polymethoxylated flavone/kg body weight of the mammal and a rate of once every two days.

13. The method of claim 8, wherein administering is by an oral route.

14. The method of claim 13, wherein the composition is in a form selected from the group consisting of liquids, tablets, capsules, caplets, and soluble powders.

15. The method of claim 8, wherein the sleep disorder is selected from the group consisting of insomnia, hypersomnia, narcolepsy, delayed sleep phase disorder (DSPD), advanced sleep phase disorder (ASPD), non-24-hour sleep-wake disorder, irregular sleep wake rhythm, and shift work sleep disorder.

16. A method, comprising:
administering, to a mammal suffering from aging, a composition comprising at least one polymethoxylated flavone.

17. The melhod of claim 16, wherein the at least one polymethoxylaied flavone has formula 1: 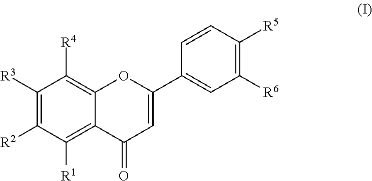
wherein R¹, R², R³, R⁴, R⁵, and R⁶ are each independently —H or —OCH₃, provided at least two of R¹, R², R³, R⁴, R⁵, and R⁶ are —OCH₃.

18. The method of claim 17, wherein the at least one polymethoxylated flavone is selected from the group consisting of nobiletin, tangeretin, and both.

19. The method of claim 16, wherein administering is at a dosage from 20 mg polymethoxylated flavone/kg body weight of the mammal to 2000 mg polymethoxylated flavone/kg body weight of the mammal and a rate from twice per day to once per week.

20. The method of claim 19, wherein administering is at a dosage of 200 mg polymethoxylated flavone/kg body weight of the mammal and a rate of once every two days.

21. The method of claim 16, wherein administering is by an oral route.

22. The method of claim 21, wherein the composition is in a form selected from the group consisting of liquids, tablets, capsules, caplets, and soluble powders
